(19) United States
(12) Patent Application Publication
(10) Pub. No.: US 2026/0114996 A1
(43) Pub. Date: Apr. 30, 2026

(54) DEVICES AND METHODS FOR CINCHING A SIDE-DELIVERED PROSTHETIC HEART VALVE FOR DELIVERY AND DEPLOYMENT IN A NATIVE ANNULUS

(71) Applicant: VDyne, Inc., Maple Grove, MN (US)

(72) Inventors: Craig EKVALL, East Bethel, MN (US); Robert VIDLUND, Forest Lake, MN (US); Mark CHRISTIANSON, Plymouth, MN (US)

(73) Assignee: VDyne, Inc., Maple Grove, MN (US)

(21) Appl. No.: 19/428,845

(22) Filed: Dec. 22, 2025

Related U.S. Application Data
(63) Continuation of application No. PCT/US2024/035388, filed on Jun. 25, 2024.
(60) Provisional application No. 63/510,754, filed on Jun. 28, 2023.
(60) Provisional application No. 63/514,421, filed on Jul. 19, 2023.

(51) Int. Cl. A61F 2/24

(52) U.S. Cl. CPC A61F 2/2436; A61F 2/2418; A61F 2220/0008

(57) ABSTRACT

A side-deliverable prosthetic valve includes a valve frame and a flow control component mounted within a central channel of the valve frame and configured to permit blood flow through the central channel in a direction along a central axis of the prosthetic valve. An actuator is removably attached to a proximal subannular anchoring element of the valve frame. A first portion of the actuator is removably attached to an interior surface of the valve frame in at least one location distal to the proximal subannular anchoring element. The first portion is configured to pull the proximal subannular anchoring element inwardly toward the central axis. A second portion of the actuator is removably attached, at least in part, to an exterior surface of the valve frame. The second portion is configured to pull the proximal subannular anchoring element outwardly away from the central axis.

### CROSS-REFERENCE TO RELATED APPLICATIONS

This application is a continuation of International Application No. PCT/US2024/035388, filed Jun. 25, 2024, entitled “Devices and Methods for Cinching a Side-Delivered Prosthetic Heart Valve for Deployment in a Native Annulus,” which claims priority to and the benefit of U.S. Provisional Ser. No. 63/514,421 , filed Jul. 19, 2023, entitled “Devices and Methods for Cinching a Side-Delivered Prosthetic Heart Valve for Delivery and Deployment in a Native Annulus,” and U.S. Provisional Ser. No. 63/510,754, filed Jun. 28, 2023, entitled “Devices and Methods for Cinching a Side-Delivered Prosthetic Heart Valve for Deployment in a Native Annulus,” the disclosure of each of which is incorporated herein by reference in its entirety.

### BACKGROUND

Embodiments described herein relate generally to transcatheter prosthetic heart valves and more particularly, to devices, systems, and/or methods for cinching side-deliverable transcatheter prosthetic heart valves during, for example, deployment into an annulus of a native valve.

Prosthetic heart valves can pose challenges for delivery, deployment, and/or retrieval within a heart, particularly for delivery by catheters through the patient's vasculature rather than through a surgical approach. Delivery of traditional transcatheter prosthetic valves generally includes compressing the valve in a radial direction and loading the valve into a delivery catheter such that a central annular axis of the valve is parallel to a lengthwise or longitudinal axis of the delivery catheter. In other words, traditional prosthetic valves are loaded into a delivery catheter such that a radial extent of the valve is aligned with and/or fits within a radial extent of a lumen extending through the delivery catheter. The valves are deployed from an end of the delivery catheter and expanded outwardly in a radial direction from the central annular axis. The vasculature of a patient, however, places limitations on a diameter of the delivery catheter, which in turn, places limits on the radial extent of the lumen extending through the delivery catheter, and thus, limits the expanded size (e.g., diameter) of a prosthetic valve delivered using the traditional, radial compressed delivery method. The competing interest of minimizing delivery catheter size presents challenges to increasing the expanded diameter of radially compressed valves (e.g., trying to compress too much material and structure into too little space). Moreover, the orientation of the traditional valves during deployment can create additional challenges when trying to align the valves with the native valve annulus.

Some transcatheter prosthetic valves can be configured for side and/or orthogonal delivery, which can allow for an increase in an expanded diameter relative to traditionally delivered valves. With side delivery, for example, the valve can be placed in a compressed or delivery configuration and loaded into a delivery catheter such that a central annular axis of the valve is substantially perpendicular and/or orthogonal to the lengthwise or longitudinal axis of the delivery catheter. More particularly, the valve can be compressed axially (e.g., along the central annular axis) and laterally (e.g., perpendicular to each of the central annular axis and a longitudinal axis of the valve), and uncompressed or elongated longitudinally (e.g., in a direction parallel to the lengthwise or longitudinal axis of the delivery catheter). The compressed valve (e.g., the valve in a delivery configuration) can be loaded into a lumen of the delivery catheter in a side-ways or orthogonal orientation (relative to an orientation of traditionally delivered valves), in which the central annular axis of the valve is substantially perpendicular and/or orthogonal to the lengthwise or longitudinal axis of the delivery catheter (e.g., the longitudinal axis of the valve is parallel to the lengthwise or longitudinal axis of the delivery catheter). Once loaded, the compressed valve can be advanced through the lumen of the delivery catheter and released from the end of the delivery catheter (e.g., into a chamber of the heart such as an atrium). Furthermore, in some instances, the side-ways or orthogonal orientation of the released side-delivered valve relative to the delivery catheter, in general, results in the valve being in a desired orientation relative to the native valve annulus.

While side delivery can allow larger valves to be delivered to the heart and/or can simplify a process of aligning or orienting the valve relative to the native annulus (as compared to traditional delivery), challenges exist with seating side-deliverable prosthetic valves in the native annulus. For example, traditional, radially compressed valves can be maintained in an at least partially compressed state while a portion of the prosthetic valve is inserted through the annulus. Once in a desired position, the prosthetic valve can be allowed to transition to a radially uncompressed state, thereby seating the traditionally delivered valve in the native annulus. On the other hand, in some tricuspid valve replacements, seating a side-deliverable prosthetic valve can include inserting a distal portion of the valve into the annulus such that a distal wall of the valve contacts a distal wall of the annulus, a distal subannular anchor is below the annulus and disposed in or near a ventricular outflow tract (RVOT), and a supra-annular portion of the valve such as an atrial cuff or the like is above the annulus. Once positioned, the valve can be pivoted relative to the annular plane to insert a proximal portion of the valve into/through the native annulus, thereby seating the valve.

In some instances, however, it may be desirable to facilitate delivery and/or deployment of the prosthetic valve by actuating and/or cinching at least a proximal subannular portion of the prosthetic valve for delivery and/or deployment of the valve into/through the native annulus. After seating the prosthetic valve, the proximal subannular portion thereof can be released, actuated, and/or otherwise allowed to return or substantially return to an unactuated, uncinched, or biased configuration. Moreover, in some instances, it may be desirable to actuate and/or cinch the proximal subannular portion of the prosthetic valve in a manner that allows for the portion of the prosthetic valve to be released while avoiding and/or limiting undesirable contact with native tissue defining the annulus that may otherwise push the proximal subannular portion of the prosthetic valve in a direction toward the atrium (e.g., away from the ventricle), resulting in an incomplete or otherwise partial seating of the prosthetic valve in the annulus.

Accordingly, a need exists for devices, systems, and/or methods for cinching side-deliverable transcatheter prosthetic heart valves during, for example, deployment into an annulus of a native valve.

### SUMMARY

The embodiments described herein are directed to side-deliverable transcatheter prosthetic heart valves and devices, systems, and/or methods for cinching side-deliverable transcatheter prosthetic heart valves during, for example, deployment into an annulus of a native valve. In some implementations, a side-deliverable prosthetic valve includes a valve frame and a flow control component mounted within a central channel extending along a central axis of the prosthetic valve. The flow control component is configured to permit blood flow through the central channel in a direction along the central axis of the prosthetic valve. An actuator is configured to be removably attached to a proximal subannular anchoring element of the valve frame. A first portion of the actuator is removably attached to an interior surface of the valve frame in at least one location distal to the proximal subannular anchoring element. The first portion is configured, in response to a proximally directed force, to pull the proximal subannular anchoring element inwardly toward the central axis to a first configuration. A second portion of the actuator is removably attached, at least in part, to an exterior surface of the valve frame. The second portion is configured, in response to a proximally directed force, to pull the proximal subannular anchoring element outwardly away from the central axis from the first configuration to a second configuration.

### BRIEF DESCRIPTION OF THE DRAWINGS

FIGS. 1-5 are schematic illustrations of a side-deliverable transcatheter prosthetic valve selectively coupled to a delivery system (or portions thereof) used to deliver and deploy the prosthetic valve into an annulus of a native heart valve, according to an embodiment.

FIG. 6 is a side-view schematic illustration of an actuator coupled to a prosthetic valve having a proximal anchoring element shown in an uncinched or expanded configuration, according to an embodiment.

FIGS. 7 and 8 are a side-view schematic illustration and a bottom-view schematic illustration, respectively, of the actuator and prosthetic valve of FIG. 6, showing the proximal anchoring element actuated to a cinched or compressed configuration.

FIG. 9 is a side-view schematic illustration of an actuator coupled to a prosthetic valve having a proximal anchoring element shown in an uncinched or expanded configuration, according to an embodiment.

FIGS. 10 and 11 are a side-view schematic illustration and a bottom-view schematic illustration, respectively, of the actuator and prosthetic valve of FIG. 9, showing the proximal anchoring element actuated to a cinched or compressed configuration.

FIG. 12 is a side-view schematic illustration of the actuator and prosthetic valve of FIG. 9, showing the proximal anchoring element actuated and/or returned to the uncinched or expanded configuration.

FIGS. 13-15 are various views of the prosthetic valve of FIG. 9 and a delivery system showing a process of loading the prosthetic valve into the delivery system while the proximal anchoring element is in the cinched or compressed configuration.

FIGS. 16 and 17 are an elevated side perspective view and a bottom perspective view, respectively, of a side-deliverable transcatheter prosthetic valve according to an embodiment.

FIG. 18 is an elevated side perspective view of a supra-annular region of an outer support frame of the prosthetic valve shown in FIG. 16.

FIG. 19 is a distal perspective view of a transannular region of the outer support frame of the prosthetic valve shown in FIG. 16.

FIG. 20 is a distal perspective view of a subannular region of the outer support frame of the prosthetic valve shown in FIG. 16.

FIG. 21 is a top perspective view of an inner frame of a flow control component included in the prosthetic valve shown in FIG. 16.

FIG. 22 is a side perspective view of a leaflet band of the inner flow control component that has leaflets sewn into a structural band, and shown in a cylindrical configuration suitable for coupling to the inner frame of FIG. 21.

FIG. 23 is a bottom view of the leaflet band of FIG. 22 in the cylindrical configuration and showing partial coaptation of the leaflets to form a partially closed fluid-seal.

FIG. 24 is an elevated side perspective view of the prosthetic valve of FIG. 16 removably coupled to a distal end portion of a control device included in a delivery system.

FIG. 25 is an elevated side perspective view of the prosthetic valve and the distal end of the control device of FIG. 24 and shown during a process of deploying the prosthetic valve into an annulus of a native heart valve.

FIGS. 26 and 27 are a schematic side view and a schematic bottom view, respectively, of a side-deliverable prosthetic valve according to an embodiment.

FIG. 28 is a schematic side view of the prosthetic valve of FIG. 26 and shown with an actuator and/or cinching assembly attached to the prosthetic valve to transition a subannular region or member of the prosthetic valve between at least a first configuration and a second configuration.

FIGS. 29-31 are front view illustrations showing a side-deliverable prosthetic valve being deployed into an annulus of a native heart valve according to an embodiment. FIG. 29 shows a subannular region or member of the prosthetic valve in an extended configuration after delivery; FIG. 30 shows the subannular region or member in a retracted or compressed configuration for seating the prosthetic valve into and/or through the native annulus; and FIG. 31 shows the subannular region or member returned (substantially) back to the extended configuration after the prosthetic valve is seated.

FIGS. 32 and 33 are bottom view illustrations showing a side-deliverable prosthetic valve, according to an embodiment, with a subannular region or member of the prosthetic valve (or valve frame thereof) removably coupled to an actuator configured to transition the subannular region or member between at least a first configuration and a second configuration to facilitate deployment of the prosthetic valve in the native annulus.

FIGS. 34-37 are bottom perspective views of a side-deliverable prosthetic valve coupled to a delivery/deployment system (or portions thereof), and showing a sequence of actuating and/or cinching one or more portions of the prosthetic valve to reduce a perimeter and/or circumference thereof to facilitate deployment of the prosthetic valve in an annulus of a native heart valve, according to an embodiment.

FIGS. 38 and 39 are bottom perspective view of a side-deliverable prosthetic valve coupled to a delivery/deployment system (or portions thereof) configured to actuate and/or cinch a subannular region or member of the prosthetic valve to facilitate deployment of the prosthetic valve in an annulus of a native heart valve, according to an embodiment.

FIGS. 40 and 41 are side views of the prosthetic valve of FIGS. 38 and 39, shown in an actuated/cinched configuration and an unactuated/uncinched configuration, respectively.

FIGS. 42 and 43 are bottom perspective view of a side-deliverable prosthetic valve coupled to a delivery/deployment system (or portions thereof) configured to actuate and/or cinch a subannular region or member of the prosthetic valve, according to an embodiment. FIG. 42 shows the prosthetic valve (or portion thereof) in an unactuated/uncinched configuration and FIG. 43 shows the prosthetic valve (or portion thereof) in an actuated/cinched configuration.

FIG. 44 is a flowchart illustrating a method of deploying a side-deliverable prosthetic valve according to an embodiment.

FIG. 45 is a flowchart illustrating a method of deploying a side-deliverable prosthetic valve according to another embodiment.

FIG. 46 is a flowchart illustrating a method of coupling an actuator to a side-deliverable prosthetic valve according to an embodiment.

### DETAILED DESCRIPTION

Disclosed embodiments are directed to side-deliverable transcatheter prosthetic heart valves and/or components thereof, and devices, systems, and/or methods for cinching side-deliverable transcatheter prosthetic heart valves during, for example, deployment into an annulus of a native valve. In some embodiments, a side-deliverable prosthetic valve includes a valve frame and a flow control component mounted within a central channel extending along a central axis of the prosthetic valve. The flow control component is configured to permit blood flow through the central channel in a direction along the central axis of the prosthetic valve. An actuator is configured to be removably attached to a proximal subannular anchoring element of the valve frame. A first portion of the actuator is removably attached to an interior surface of the valve frame in at least one location distal to the proximal subannular anchoring element. The first portion is configured, in response to a proximally directed force, to pull the proximal subannular anchoring element inwardly toward the central axis to a first configuration. A second portion of the actuator is removably attached, at least in part, to an exterior surface of the valve frame. The second portion is configured, in response to a proximally directed force, to pull the proximal subannular anchoring element outwardly away from the central axis from the first configuration to a second configuration.

In some embodiments, a side-deliverable prosthetic valve includes a valve frame having a supra-annular member, a subannular member, and a transannular member coupled therebetween. The transannular member includes a set of wire struts that define a set of diamond-shaped cells. In some implementations, a method of coupling an actuator to the valve frame of the prosthetic valve includes inserting a tether of the actuator through a waypoint defined by the supra-annular member. The tether is removably attached to a set of attachment points mounted to an interior surface of the transannular member along a strut from the set of struts. The tether is also removably attached to a proximal subannular anchoring element formed, at least in part, by the subannular member of the valve frame. A distal end portion of the tether is disposed about a guidewire catheter extending through the waypoint of the supra-annular member, and the guidewire catheter is then inserted through a guidewire coupler mounted to a distal portion of the subannular member to temporarily secure the distal end portion of the tether to the distal portion of the subannular member.

In some embodiments, a side-deliverable prosthetic valve includes (i) a valve frame having a supra-annular member, a subannular member, and a transannular member coupled therebetween, and (ii) a flow control component mounted to the valve frame and at least partially disposed in the transannular member. In some implementations, a method of deploying the prosthetic valve in an annulus of a native heart valve includes removably coupling the valve frame to a portion of a delivery system. A first force is exerted on a first portion of an actuator to pull a proximal subannular anchoring element of the subannular member inwardly to a first configuration. The prosthetic valve is advanced, in a delivery configuration, through a lumen of a delivery catheter included in the delivery system. The prosthetic valve is released from a distal end of the delivery catheter disposed in an atrium of a heart and is seated in the annulus of the native heart valve while the proximal subannular anchoring element is in the first configuration. After seating the prosthetic valve in the annulus, the method includes exerting a second force on a second portion of the actuator to pull the proximal subannular anchoring element outwardly from the first configuration to a second configuration.

In some implementations, a method of deploying a side-deliverable prosthetic heart valve in an annulus of a native heart valve includes removably coupling a portion of a delivery system to a frame of the valve. The prosthetic valve in a delivery configuration is advanced through a lumen of a delivery catheter included in the delivery system. The prosthetic valve is released from a distal end of the delivery catheter that is disposed in an atrium of the heart. The prosthetic valve is seated in the annulus of the native heart valve while a proximal anchoring element of the frame of the valve is in a first configuration. After seating the prosthetic valve, the proximal anchoring element is transitioned from the first configuration to a second configuration to secure the valve in the annulus.

In some implementations, the method may include placing the proximal anchoring element in the first configuration after releasing the prosthetic valve. In some implementations, the method may include cinching the proximal anchoring element to place the proximal anchoring element in the first configuration and compressing the prosthetic valve after cinching to place the prosthetic valve in the delivery configuration. In such implementations, the prosthetic valve in the delivery configuration may be loaded into the lumen of the delivery catheter such that a longitudinal axis of the prosthetic valve is substantially parallel to a longitudinal axis of the delivery catheter.

In some implementations, the portion of the delivery system includes an actuator and/or control device. A tether of the actuator/control device is removably coupled to the proximal anchoring element and is configured to actuate the proximal anchoring element between the first configuration and the second configuration in response to a proximally directed force exerted on the tether. In some embodiments, the prosthetic valve includes a transannular member coupled between a supra-annular member and a subannular member. The transannular member can include a wireframe that forms a number of wire cells, with each wire cell being diamond-shaped and having an orientation and cell geometry configured to allow axial compression of the valve to the delivery configuration. In some implementations, placing the proximal anchoring element of the subannular member in the first configuration includes actuating the proximal anchoring element to place the proximal anchoring element in the first configuration substantially without compressing the wire cells of the transannular member in an axial direction.

In some implementations, the actuator/control device includes one or more additional tethers that can be removably coupled to the proximal anchoring element. In such implementations, the tether (e.g., a first tether) can be configured to actuate and/or cinch the proximal anchoring element to place the proximal anchoring element in the first configuration. The method can further include increasing a tension along a second tether after seating the prosthetic valve in the annulus to transition the proximal anchoring element from the first, cinched, and/or distal configuration to the second, uncinched, and/or proximal configuration. In some implementations, the actuator and/or the second tether can include a lock or the like that can engage a portion of the outer frame, thereby locking the proximal anchoring element in the second configuration.

In some implementations, the method can further include inserting a guidewire catheter though a waypoint defined by the supra-annular member of the valve and a guidewire coupler of the subannular member of the valve. The tether of the actuator/control device is routed through a set of attachment points along a strut of the transannular member of the valve and an attachment point on the proximal anchoring element. A loop at a distal end portion of the tether is disposed about a portion of the guidewire catheter distal to the guidewire coupler to temporarily anchor the distal end portion of the tether to the subannular member of the valve. In some implementations, the method further includes retracting the guidewire catheter after seating the prosthetic valve in the annulus and transitioning the proximal anchoring element from the first configuration to the second configuration. The tether is released as a result of the retracting the guidewire catheter and is decoupled from the proximal anchoring element.

In some implementations, a method of delivering and/or deploying a side-deliverable prosthetic heart valve in an annulus of a native heart valve includes removably coupling a portion of a delivery system to an outer frame of the valve. A proximal subannular anchoring element of the outer frame is cinched to place the proximal subannular anchoring element in a first configuration. After cinching, the valve in a delivery configured is advanced through a lumen of a delivery catheter included in the delivery system. The prosthetic valve is released from a distal end of the delivery catheter that is disposed in an atrium of the heart. The prosthetic valve is seated in the annulus of the native heart valve while the proximal subannular anchoring element is in the first configuration. After seating the prosthetic valve, the proximal subannular anchoring element is transitioned from the first configuration to a second configuration to secure the valve in the annulus.

Any of the prosthetic valves described herein can be relatively low profile, side-deliverable, transcatheter prosthetic heart valves (also referred to herein as “prosthetic valve” or simply, “valve”). The prosthetic valves herein can have a valve frame and a flow control component mounted within a central lumen, aperture, and/or channel of the valve frame that extends along a central axis of the valve or valve frame that is co-axial or at least substantially parallel with a blood flow direction through the valves. The valve frame can provide structural support for the prosthetic valve and/or at least the flow control component mounted thereto. The valve frame can also provide one or more components or elements for anchoring or otherwise securing the prosthetic valves in an annulus of a native valve. The flow control component (e.g., a 2-leaflet or 3-leaflet sleeve, valve, and/or the like) can be configured to permit blood flow in a first direction through an inflow end of the valve and out an outflow end of the valve, and block blood flow in a second direction, opposite the first direction.

The prosthetic valves described herein are configured for transcatheter delivery to a chamber of the heart. For example, the prosthetic valves can transition or can be transitioned (e.g., via balloon inflation or via one or more self-expanding structures) between a compressed or delivery configuration for introduction into the body via a delivery catheter (e.g., a 24-36 French (Fr) delivery catheter), and an expanded or deployment/deployed configuration for implanting and/or deploying into a native heart valve. More specifically, the prosthetic valves described herein are configured for orthogonal or side transcatheter delivery to the heart as opposed to traditional transcatheter delivery.

In general, valves delivered via traditional transcatheter delivery are configured to be compressed in, for example, a radial direction relative to a central axis or blood flow direction through the valve, and inserted into and/or advanced through the delivery catheter such that the central axis of the compressed valve is parallel to a longitudinal or lengthwise axis of the delivery catheter used to deliver the valve. The valves are deployed from the end of the delivery catheter and expanded or allowed to expand outwardly in a radial direction from the central cylinder axis. The delivery orientation of the valve generally means that the valve is completely released from the delivery catheter while in the atrium of the heart and is then reoriented relative to the annulus, which in some instances, can limit a size of the valve. Accordingly, in some implementations, traditional delivery can be used for relatively small diameter valves such as, for example, prosthetic pulmonary and/or aortic valves.

Orthogonal or side-delivered/deliverable valves are configured to be compressed in at least one of a lateral direction (orthogonal to the blood flow direction through the valve) or an axial direction (parallel to or aligned with the blood flow direction). In some embodiments, any of the valves can be compressed in two directions—the lateral direction and the axial direction—without compressing the valve in a direction along a lengthwise or longitudinal axis of the valve (orthogonal to the blood flow direction through the valve). With orthogonal or side-delivery, the compressed valve can be inserted and/or advanced through a delivery catheter such that the central axis of the compressed valve is substantially orthogonal, perpendicular, and/or sideways relative to a longitudinal or lengthwise axis of the delivery catheter. Said another way, in orthogonal or side-delivery, the lengthwise or longitudinal axis of the valve can be substantially parallel to the lengthwise or longitudinal axis of the delivery catheter through which the valve is delivered. Thus, an orthogonally delivered and/or side delivered prosthetic valve is compressed and/or delivered sideways (e.g., at a roughly 90-degree angle) compared to traditional processes of compressing and delivering transcatheter prosthetic valves.

In some implementations, orthogonal or side delivery of prosthetic valves can allow the valves to be deployed from the inferior vena cava (IVC) into the annulus of a native mitral or tricuspid valve without a need for substantial reorientation of the valve or without a need to position the delivery catheter at an acute angle relative to the native valve (that is otherwise common in traditional transcatheter delivery). In addition, the orientation of orthogonal or side deliverable prosthetic valves relative to the annulus can allow a distal portion of the valve to be at least partially inserted into the annulus of the native heart valve while the proximal portion of the valve, at least in part, remains in the delivery catheter, thereby avoiding at least some of the size constraints faced with some known traditional delivery techniques. For example, a relatively large side-deliverable prosthetic valve in an expanded configuration can have a height of about 5-60 millimeters (mm) and a diameter of about 20-80 mm, and in a compressed configuration can have a height of about 5-12 mm, a width (e.g., in a lateral direction) of about 8-12 mm, and a length (e.g., in a longitudinal or lengthwise direction) of about 25-80 mm.

While valves configured for orthogonal delivery can allow for the deployment of relatively large valves, traditional, radially compressible valves can be maintained in an at least partially compressed configuration and/or state during delivery and deployment, which in some instances, may facilitate the process of seating some traditionally delivered prosthetic valves in the annulus of a native heart valve. For example, such valves can be at least partially radially compressed to allow a portion of the prosthetic valve to be dropped into the annulus (e.g., compressed such that a diameter and/or circumference of at least a portion of the prosthetic valve is less than a diameter and/or circumference of the native annulus). Once in a desired position, the prosthetic valve can be transitioned and/or allowed to transition to a radially expanded or radially uncompressed state, thereby seating the prosthetic valve in the annulus of the native heart valve.

On the other hand, the process of deploying and/or seating certain orthogonally delivered prosthetic valves can include inserting a distal portion of the prosthetic valve through the annulus and then pivoting the remaining portion(s) of the valve into a desired position. In some instances, this difference in the process of seating the valve in the annulus can give rise to a desire for additional features and/or methods that facilitate deployment (seating) of the orthogonally delivered valve into the native annulus. For example, it may be desirable to actuate and/or cinch one or more portions of the prosthetic valve to allow the valve to be pivoted or dropped into the annulus. In some embodiments, the actuation and/or cinching can be similar to or part of the process for transitioning the prosthetic valve in the compressed or delivery configuration, or it can be independent of the process for transitioning the prosthetic valve in the compressed or delivery configuration. Accordingly, the embodiments and/or methods described herein relate to delivery/deployment systems configured to selectively actuate and/or cinch one or more portions of a side-deliverable prosthetic valve to facilitate deployment into the native annulus.

Any of the prosthetic heart valves described herein can include an outer support frame that includes and/or forms a supra-annular region, a subannular region, and a transannular region coupled therebetween. The supra-annular region can form, for example, an upper collar portion of the outer support frame and can include any number of features configured to engage native tissue, an inner flow control component of the prosthetic valve, and/or a delivery, actuator, and/or retrieval mechanism. The subannular region can form, for example, one or more anchoring elements configured to engage subannular (ventricular) tissue when the prosthetic valve is seated in the native annulus. The transannular region can be coupled between the supra-annular region and the subannular region. The transannular region can form a shape such as a funnel, cylinder, flat cone, or circular hyperboloid when the outer support frame is in an expanded configuration.

In some embodiments, the outer support frame includes and/or is at least partially formed from a wire, a braided wire, or a laser-cut wire frame, and is at least partially covered with a biocompatible material. For example, the outer support frame and/or at least the transannular region thereof can include and/or form a set of compressible wire cells such as braided-wire cells, laser-cut wire cells, photolithography produced wire cells, 3D printed wire cells, wire cells formed from intermittently connected single strand wires in a wave shape, a zig-zag shape, or spiral shape, and/or combinations thereof. In some implementations the compressible wire cells can have an orientation and cell geometry that is substantially orthogonal to the central axis to minimize wire cell strain when the outer support frame is in a delivery configuration (e.g., a compressed, rolled, and/or folded configuration).

Any of the prosthetic heart valves described herein (and/or outer frames thereof) can include a single anchoring element or multiple anchoring elements configured to anchor the valve in the annulus of a native valve (e.g., subannular anchoring elements, supra-annular anchoring elements, and/or a combination thereof). For example, in some implementations, a prosthetic valve and/or outer frame can include one or more of a distal subannular anchoring element configured to engage ventricular tissue distal to the annulus (e.g., can extend into a right ventricular outflow tract (RVOT)); a proximal subannular anchoring element configured to engage ventricular tissue proximal to the annulus (e.g., between the septal leaflets and the posterior leaflets of the heart); a septal anchoring element configured to engage at least one of a native septal wall or a native septal leaflet when the prosthetic heart valve is seated in the annulus (e.g., to pin at least the native septal leaflet away from the coapting leaflets of the prosthetic valve); and/or any other suitable anchoring element. In some implementations, one or more of the subannular anchoring elements can stabilize the valve against intra-annular rolling forces and/or twisting forces that might affect a desired location or positioning of the prosthetic valve within the annulus, (e.g., tilted, angled, twisted, rolled, etc.). Moreover, any of the prosthetic valves described herein can include subannular portion(s) and/or anchoring element(s) that can be actuated and/or otherwise transitioned between two or more configurations to facilitate deployment of the prosthetic valves in the annulus, as described in further detail herein with respect to specific embodiments.

Any of the prosthetic valves and/or outer frames thereof can also include, for example, a distal and/or proximal upper anchoring element configured to be positioned into a supra-annular position in contact with and/or adjacent to supra-annular tissue of the atrium (e.g., right atrium). In some implementations, the upper anchoring element(s) can be configured to exert a force on supra-annular tissue and the lower anchoring element(s) can be configured to exert a force in an opposite direction on subannular tissue, thereby securing the prosthetic valve in the native annulus.

Any of the prosthetic valves described herein can include an inner flow control component that has a leaflet frame with 2-4 flexible leaflets mounted thereon. The 2-4 leaflets are configured to permit blood flow in a first direction through an inflow end of the valve and out an outflow end of the valve, and block blood flow in a second direction, opposite the first direction. The leaflet frame can include any number of panels or walls of diamond-shaped or eye-shaped wire cells made from heat-set shape memory alloy material such as, for example, nickel-titanium alloys (e.g., Nitinol®). The leaflet frame can be configured to be foldable along a z-axis (e.g., a longitudinal axis) from a rounded or cylindrical configuration to a flattened cylinder configuration, and compressible along a vertical y-axis (e.g., a central axis) to a compressed configuration. In some implementations, the leaflet frame can include a pair of hinge areas, fold areas, connection points, etc. that can allow the leaflet frame to be folded flat along the z-axis prior to the leaflet frame being compressed along the vertical y-axis. The leaflet frame can be, for example, a single-piece structure with two or more living hinges (e.g., stress concentration riser(s) and/or any suitable structure configured to allow for elastic/nonpermanent deformation of the leaflet frame) or a two-piece structure where the hinge areas are formed using a secondary attachment method (e.g., sutures, fabrics, molded polymer components, etc.). In some embodiments, the inner flow control component in an expanded configuration forms a shape such as a funnel, cylinder, flat cone, or circular hyperboloid. In some embodiments, the inner flow control component has a leaflet frame with a side profile of a flat cone shape having an outer diameter R of about 20-60 mm, an inner diameter r of about 10-50 mm, where diameter R is greater than diameter r, and a height of about 5-60 mm. In some embodiments, the leaflet frame is comprised of a wire, a braided wire, or a laser-cut wire frame.

Any of the prosthetic valves and/or components thereof may be fabricated from any suitable biocompatible material or combination of biocompatible materials. For example, an outer valve frame, an inner valve frame (e.g., of an inner flow control component), and/or components thereof may be fabricated from biocompatible metals, metal alloys, polymer coated metals, and/or the like. Suitable biocompatible metals and/or metal alloys can include stainless steel (e.g., 316 L stainless steel), cobalt chromium (Co—Cr) alloys, nickel-titanium alloys (e.g., Nitinol®), and/or the like. Moreover, any of the outer or inner frames described herein can be formed from superelastic or shape-memory alloys such as nickel-titanium alloys (e.g., Nitinol®). Synthetic biocompatible materials can include, for example, polyesters, polyurethanes, elastomers, thermoplastics, thermoplastic polycarbonate urethane, polyether urethane, segmented polyether urethane, silicone polyether urethane, polyetheretherketone (PEEK), silicone-polycarbonate urethane, polypropylene, polyethylene, low-density polyethylene (LDPE), high-density polyethylene (HDPE), ultra-high density polyethylene (UHDPE), polyolefins, polyethylene-glycols, polyethersulphones, polysulphones, polyvinylpyrrolidones, polyvinylchlorides, other fluoropolymers, polyesters, polyethylene-terephthalate (PET) (e.g., Dacron®), Poly-L-lactic acids (PLLA), polyglycolic acid (PGA), poly(D, L-lactide/glycolide) copolymer (PDLA), silicone polyesters, polyamides (Nylon), polytetrafluoroethylene (PTFE) (e.g., Teflon), elongated PTFE, expanded PTFE, siloxane polymers and/or oligomers, polylactones, and/or the like or block co-polymers using the same.

Any of the prosthetic valves and/or components thereof can include and/or can be formed with one or more biocompatible coating(s) and/or the like. Suitable polymer coatings can include, for example, polyethylene vinyl acetate (PEVA), poly-butyl methacrylate (PBMA), translute Styrene Isoprene Butadiene (SIBS) copolymer, polylactic acid, polyester, polylactide, D-lactic polylactic acid (DLPLA), polylactic-co-glycolic acid (PLGA), and/or the like. Some such polymer coatings may form a suitable carrier matrix for drugs such as, for example, Sirolimus, Zotarolimus, Biolimus, Novolimus, Tacrolimus, Paclitaxel, Probucol, and/or the like.

Any of the outer valve frames, inner flow control frames, and/or portions or components thereof can be internally or externally covered, partially or completely, with a natural or synthetic biocompatible and/or biological material such as pericardium, or the like. For example, where a thin, durable synthetic material is contemplated (e.g., for a covering), synthetic polymer materials such expanded PTFE, PET, or polyester (or any of the other materials described herein) may optionally be used. Suitable biological material or tissue used for coverings (or the like) can include, for example, chemically stabilized pericardial tissue of an animal, such as a cow (bovine pericardium), sheep (ovine pericardium), pig (porcine pericardium), or horse (equine pericardium). For example, suitable tissue may include, but is not limited to, tissue used in the products Duraguard®, Peri-Guard®, and Vascu-Guard®, all products currently used in surgical procedures, products which are marketed as being harvested generally from cattle less than 30 months old, and/or the like. In some implementations, a valve can be configured such that an inner surface of the outer valve frame (e.g., the wireframe cells) is covered with pericardial tissue, and an outer surface is covered with a woven synthetic polyester material such as Dacron® (or vice versa), or both the inner surface and outer surface is covered with pericardial tissue or a woven synthetic polyester material.

In some embodiments, a side-deliverable prosthetic heart valve has an outer frame with a supra-annular member, a subannular member, and a transannular member coupled therebetween and a flow control component mounted to the outer frame and at least partially disposed in the transannular member. In some implementations, a method of deploying the prosthetic valve in an annulus of a native heart valve includes removably coupling the outer frame to a portion of a delivery system. The prosthetic valve in a delivery configuration is advanced through a lumen of a delivery catheter included in the delivery system. The delivery catheter has a distal end that is disposed in an atrium of the heart as the prosthetic valve is advanced. The prosthetic valve is released from the distal end of the delivery catheter and seated in the annulus of the native heart valve while a proximal anchoring element of the subannular member of the outer frame is in a first or cinched configuration. The proximal anchoring element is then transitioned and/or otherwise allowed to transition from the first or cinched configuration to and/or toward a second uncinched configuration after seating the prosthetic valve in the annulus. The proximal anchoring element may be biased or pre-disposed in the second configuration and can be actuated, cinched, placed, and/or otherwise transitioned to the first configuration prior to delivery or after being released from the delivery catheter (e.g., after the prosthetic valve is released but prior to the prosthetic valve being seated in the native annulus).

Any method for delivering and/or deploying prosthetic heart valves described herein can include delivery of the prosthetic heart valve to a native annulus of a human heart that includes advancing a delivery catheter to at least one of (i) the tricuspid valve or pulmonary artery of the heart through the inferior vena cava (IVC) via the femoral vein or through the superior vena cava (SVC) via the jugular vein, or (ii) the mitral valve or aortic valve of the heart through a trans-atrial approach (e.g., fossa ovalis or lower), via the IVC-femoral or the SVC-jugular approach. The prosthetic valve(s) is/are removably coupled to a portion of the delivery system, placed into a compressed or delivery configuration (e.g., for orthogonal and/or side delivery), loaded into a delivery device and/or the delivery catheter, and advanced through a lumen of the delivery catheter. The prosthetic valve(s) can then be released from a distal end of the delivery catheter, which is disposed in an atrium of the heart using the IVC-femoral or the SVC-jugular approach. The prosthetic valve(s) is/are allowed to transition to an expanded or released configuration when released from the delivery catheter.

Any method for delivering and/or deploying prosthetic valves described herein can include positioning the valve or a portion thereof in a desired position relative to the native tissue. For example, a method can include inserting a distal subannular anchoring element of a prosthetic valve through an annulus of the native tricuspid valve and into, for example, the RVOT of the right ventricle. In some implementations, the method can include partially inserting a prosthetic valve into the annulus (e.g., of the native tricuspid valve) such that a distal portion thereof contacts native annular tissue while a proximal portion of the prosthetic valve is at least partially compressed and disposed in the delivery catheter. In some embodiments, the method can include rotating the prosthetic heart valve, using a steerable control catheter, a yoke, a set of tethers, an actuator, and/or any other portion of a delivery/deployment system (or combinations thereof), along an axis parallel to the plane of the valve annulus. In some embodiments, the method can include transitioning one or more anchoring elements into a desired position and/or state to engage native tissue surrounding at least a portion of the annulus. In some implementations, one or more tissue anchors may be attached to the valve and to native tissue to secure the valve in a desired position.

Any of the delivery/deployment systems described herein can include an outer catheter (e.g., a delivery catheter), a control catheter, and/or other suitable portion(s) that can include one or more members, components, features, and/or the like configured to facilitate delivery and/or deployment of the valve into an annulus of a native heart valve. For example, in some implementations, a delivery/deployment system can include any number of actuators, control devices, supports, and/or the like that can at least temporarily couple to the prosthetic valve to support, stabilize, actuate, cinch and/or otherwise control one or more portions of the prosthetic valve, for example, during deployment. In some implementations, such devices and/or features can be and/or can include tethers, sutures, tensile or tension members, rods, cables, wires, catheters, hypotubes, connectors, couplers, etc. In such implementations, the devices and/or features can engage one or more portions of the prosthetic valve to support, stabilize, actuate, cinch, and/or otherwise control the prosthetic valve (e.g., during deployment) and then can be decoupled and/or removed from the prosthetic valve once it is seated in the annulus of the native valve in a desired manner, orientation, etc. For example, certain embodiments described herein can include one or more actuators, devices, and/or features that is/are configured to removably couple to a subannular portion of the prosthetic valve to at least partially actuate, cinch, control, etc. the prosthetic valve and/or at least one or more portions thereof.

The terminology used herein is for the purpose of describing particular embodiments, implementations, and/or concepts (including any feature(s) or aspect(s) thereof) and is not intended to be limiting. Unless defined otherwise, all technical and/or scientific terms used herein have the same meanings as commonly understood by one of ordinary skill in the art. Any explanation, discussion, or use of particular terms is intended to provide context and to facilitate understanding and is not necessarily intended to replace or supersede commonly used or known definitions understood by one skilled in the art unless explicitly stated otherwise. Moreover, various terms may be used to describe similar or substantially the same embodiments, implementations, and/or concepts (including any feature(s) or aspect(s) thereof) and thus, the use of particular terms is not intended to be limiting and/or to the exclusion of other terms unless the terms are mutually exclusive, or the context clearly states otherwise.

As used herein, the singular forms “a,” “an,” and “the” are intended to include the plural forms as well, unless the context clearly indicates otherwise. With respect to the use of substantially any plural and/or singular terms herein, those having skill in the art can translate from the plural to the singular and/or from the singular to the plural as is appropriate to the context and/or application. Furthermore, any reference herein to a singular component, feature, aspect, etc. is not intended to imply the exclusion of more than one such component, feature, aspect, etc. (and/or vice versa) unless expressly stated otherwise. The various singular/plural permutations may be expressly set forth herein for sake of clarity.

In general, terms used herein and in the appended claims are intended as “open” terms unless explicitly stated otherwise. For example, the term “including” should be interpreted as “including but not limited to,” the term “having” should be interpreted as “having at least,” etc. Similarly, the terms “comprises” and/or “comprising” may specify the presence of stated features, elements, components, integers (or fractions thereof), steps, operations, and/or the like but do not preclude the presence or addition of one or more other features, elements, components, integers (or fractions thereof), steps, operations, and/or groups thereof, and/or the like unless such combinations are otherwise mutually exclusive.”

As used herein the term “and/or” includes any and all combinations of one or more of the associated listed items. It should be understood that any suitable disjunctive word and/or phrase presenting two or more alternative terms, whether in the written description or claims, contemplate the possibilities of including one of the terms, either of the terms, or both/all of the terms. For example, the phrase “A and/or B” will be understood to include the possibilities of “A” alone, “B” alone, or a combination of “A and B.”

All ranges disclosed herein also encompass any and all possible subranges and combinations of subranges thereof unless expressly stated otherwise. Any listed range should be recognized as sufficiently describing and enabling the same range being broken down into at least equal subparts unless expressly stated otherwise. As will be understood by one skilled in the art, a range includes each individual member.

As used herein, the terms “about,” “approximately,” and/or “substantially” when used in connection with stated value(s) and/or geometric structure(s) or relationship(s) is intended to convey that the value or characteristic so defined is nominally the value stated or characteristic described. In some instances, the terms “about,” “approximately,” and/or “substantially” can generally mean and/or can generally contemplate a value or characteristic stated within a desirable tolerance (e.g., plus or minus 10% of the value or characteristic stated). For example, a value of about 0.01 can include 0.009 and 0.011, a value of about 0.5 can include 0.45 and 0.55, a value of about 10 can include 9 to 11, and a value of about 1000 can include 900 to 1100. Similarly, a first surface may be described as being substantially parallel to a second surface when the surfaces are nominally parallel. While a value, structure, and/or relationship stated may be desirable, it should be understood that some variance may occur as a result of, for example, manufacturing tolerances or other practical considerations (such as, for example, the pressure or force applied through a portion of a device, conduit, lumen, etc.). Accordingly, the terms “about,” “approximately,” and/or “substantially” can be used herein to account for such tolerances and/or considerations.

The terms “prosthetic heart valve,” and/or “prosthetic valve” can refer to a combination of a frame and a leaflet or flow control structure or component, and can encompass both complete replacement of an anatomical part (e.g., a new mechanical valve replaces a native valve), as well as medical devices that take the place of and/or assist, repair, or improve existing anatomical parts (e.g., the native valve is left in place). As used herein, the term “valve” may be used to refer to either a “prosthetic valve” or a “native valve,” and will be understood within the specific context in which the term is used.

Prosthetic valves disclosed herein can include a member (e.g., a “frame”) that can be seated within a native valve annulus and can be used as a mounting element for a leaflet structure, a flow control component, or a flexible reciprocating sleeve or sleeve-valve. Such a member may or may not include such a leaflet structure or flow control component, depending on the embodiment. Such members can be referred to herein as an “annular support frame,” “wire frame,”“valve frame,”“flange,”“collar,”“cuff,”and/or any other similar terms.

The term “flow control component” can refer in a non-limiting sense to a leaflet structure having 2-, 3-, 4-leaflets of flexible biocompatible material such a treated or untreated pericardium that can be sewn, joined, and/or mounted to an annular support frame, to function as a prosthetic heart valve. Such a valve can be a heart valve, such as a tricuspid, mitral, aortic, or pulmonary, that is open to blood flowing during diastole from atrium to ventricle, and that closes from systolic ventricular pressure applied to the outer surface. Repeated opening and closing in sequence can be described as “reciprocating.” The flow control component is contemplated to include a wide variety of (bio)prosthetic artificial heart valves and/or components. For example, such (bio)prosthetics can include ball valves (e.g., Starr-Edwards), bileaflet valves (St. Jude), tilting disc valves (e.g., Bjork-Shiley), stented pericardium heart valves (bovine, porcine, ovine) (Edwards'line of bioprostheses, St. Jude prosthetic valves), as well as homograft and autograft valves. Bioprosthetic pericardial valves can include bioprosthetic aortic valves, bioprosthetic mitral valves, bioprosthetic tricuspid valves, and bioprosthetic pulmonary valves.

The terms “anchoring element” or “tab” or “arm” refer to structural elements extending from a portion of the valve or valve frame (e.g., extending away from a valve sidewall, body, or collar) to provide an anchoring or stabilizing function to the valve. In some implementations, the anchoring element(s), tab(s), arm(s), etc. can include and/or can be formed from a wire loop or wire frame, an integrated frame section, a stent, and/or any other suitable structure that extends from the frame (e.g., about 10-40 mm away from a perimeter of at least a corresponding portion of the frame). When used in conjunction with the terms distal, proximal, septal, anterior, supra-annular, subannular, etc., it should be understood that the anchoring or stabilizing element so described is attached to and/or integral with the valve (or valve frame) at or near such a location. A distal location on a valve refers to a portion of the valve furthest from the practitioner which exits the delivery catheter first, and which can be placed at or near distal subannular native tissue such as the ventricular outflow tract. A proximal location on a valve refers to a portion of the valve closest to the practitioner which exits the delivery catheter last, and which can be placed at or near proximal subannular native tissue such as tissue closest to the inferior vena cava. A septal location on a valve refers to a portion of the valve at a point between a proximal and a distal location, and which can be placed at or near septal subannular native tissue such as the septal leaflet or septal wall. An anterior location on a valve refers to a portion of the valve at a point between a proximal and a distal location, and which can be placed at or near anterior tissue opposite the septal tissue. When used in conjunction with the term “lower,” or “subannular” it should be understood that the anchoring or stabilizing element so described is attached to and/or integral with the valve sidewall, body, and/or frame at or along a lower or subannular region of the valve. Conversely, when used in conjunction with the term “upper,” or “supra-annular” it should be understood that the anchoring or stabilizing element so described is attached to and/or integral with the valve or frame at or along a supra-annular region, collar, or atrial cuff of the valve.

Any of the disclosed valve embodiments may be delivered by a transcatheter approach. The term “transcatheter” is used to define the process of accessing, controlling, and/or delivering a medical device or instrument within the lumen of a catheter that is deployed into a heart chamber (or other desired location in the body), as well as an item that has been delivered or controlled by such as process. Transcatheter access is known to include cardiac access via the lumen of the femoral artery and/or vein and IVC, via the lumen of the brachial artery and/or vein, via lumen of the carotid artery, via the lumen of the jugular vein and SVC, via the intercostal (rib) and/or sub-xiphoid space, and/or the like. Moreover, transcatheter cardiac access can also include a trans-atrial (e.g., fossa ovalis or lower) approach to the left atrium and/or ventricle. Transcatheter can be synonymous with transluminal and is functionally related to the term “percutaneous”as it relates to delivery of heart valves.

As used herein the terms “orthogonal delivery,” “orthogonally delivered,” “side-delivery,” “side-delivered,” “side-deliverable,” and/or so forth can be used interchangeably to describe such a delivery method and/or a valve delivered using such a method. The term “orthogonal” refers to an intersecting angle of 90 degrees between two lines or planes (e.g., the two lines or planes are perpendicular). As used herein, the term “substantially orthogonal” refers to an intersecting angle of 90 degrees plus or minus a suitable tolerance. For example, “substantially orthogonal” can refer to an intersecting angle ranging from 75 to 105 degrees. Orthogonal and/or side delivery of prosthetic valves can be such that the central axis of the valve is substantially orthogonal to the lengthwise or longitudinal axis of the delivery catheter through which it is delivered (e.g., the valve is oriented sideways relative to traditional, radially compressed valves).

The mode of cardiac access can be based at least in part on a “body channel,” used to define a blood conduit or vessel within the body, and the particular application of the disclosed embodiments of prosthetic valves can determine the body channel at issue. An aortic valve replacement, for example, would be implanted in, or adjacent to, the aortic annulus. Likewise, a tricuspid or mitral valve replacement would be implanted at the tricuspid or mitral annulus, respectively. While certain features described herein may be particularly advantageous for a given implantation site, unless the combination of features is structurally impossible or excluded by claim language, any of the valve embodiments described herein could be implanted in any body channel.

The terms “expandable” and/or “compressible” as used herein may refer to a prosthetic heart valve or a component of the prosthetic heart valve capable of expanding and/or compressing from a first size or configuration to a second size or configuration. For example, a prosthetic valve may be “compressible” to a delivery size or configuration and/or “expandable” to an implantation or deployment size or configuration. Therefore, unless the context clearly indicates otherwise, an “expandable”/“compressible” structure is not intended to refer to a structure that might undergo slight expansion/compression such as, for example, from a change in temperature or other such incidental cause. Conversely, “non-expandable”/“non-compressible” should not be interpreted to mean completely rigid or a dimensionally stable, as some slight expansion/compression of conventional “non-expandable”/“non-compressible” heart valves, for example, may be observed.

The prosthetic valves disclosed herein and/or components thereof are generally capable of transitioning between two or more configurations, states, shapes, and/or arrangements. For example, prosthetic valves described herein can be compressible and/or expandable between any suitable number of configurations. Various terms can be used to describe or refer to these configurations and are not intended to be limiting unless the context clearly states otherwise. For example, a prosthetic valve can be described as being placed in a “delivery configuration,” which may be any suitable configuration that allows or enables delivery of the prosthetic valve. Examples of delivery configurations can include a compressed configuration, a folded configuration, a rolled configuration, and/or similar configuration or any suitable combinations thereof. Similarly, a prosthetic valve can be described as being placed in an “expanded configuration,” which may be any suitable configuration that is not expressly intended for delivery of the prosthetic valve. Examples of expanded configuration can include a released configuration, a relaxed configuration, a deployed configuration, a non-delivery configuration, and/or similar configurations or any suitable combinations thereof. Some prosthetic valves described herein and/or components or features thereof can have a number of additional configurations that can be associated with various modes, levels, states, and/or portions of actuation, deployment, engagement, etc. Examples of such configurations can include an actuated configuration, a seated configuration, a secured configuration, an engaged configuration, and/or similar configurations or any suitable combinations thereof.

The embodiments, methods, and/or implementations herein, and/or the various features or advantageous details thereof, are explained more fully with reference to the non-limiting examples illustrated in the accompanying drawings and detailed in the following description. The examples and/or embodiments described herein are intended to facilitate an understanding of structures, functions, and/or aspects of the embodiments, ways in which the embodiments may be practiced, and/or to further enable those skilled in the art to practice the embodiments herein. Similarly, methods and/or ways of using the embodiments described herein are provided by way of example only and not limitation. Specific uses described herein are not provided to the exclusion of other uses unless the context expressly states otherwise. Specific examples, embodiments, methods, and/or uses described herein should not be construed as limiting the scope of the inventions or inventive concepts herein. Rather, examples and embodiments are provided so that this disclosure will be thorough and complete, and will fully convey the scope of the inventive concepts to those skilled in the art.

For example, any of the prosthetic valves described herein can be used to replace a native valve of a human heart including, for example, a mitral valve, a tricuspid valve, an aortic valve, and/or a pulmonary valve. While some prosthetic valves are described herein in the context of replacing a native mitral valve or a native tricuspid valve, it should be understood that such a prosthetic valve can be used to replace any native valve unless expressly stated otherwise or unless one skilled in the art would clearly recognize that one or more components and/or features would otherwise make the prosthetic valve incompatible for such use.

A discussion of various embodiments, components, and/or features of prosthetic valve(s) (e.g., side-deliverable, transcatheter prosthetic heart valves) is followed by a discussion of delivery/deployment systems and methods of using such systems to deliver and/or deploy a prosthetic valve into an annulus of a native heart valve. Descriptions of well-known components and processing techniques may be omitted so as to not obscure the embodiments herein. Like numbers refer to like elements throughout.

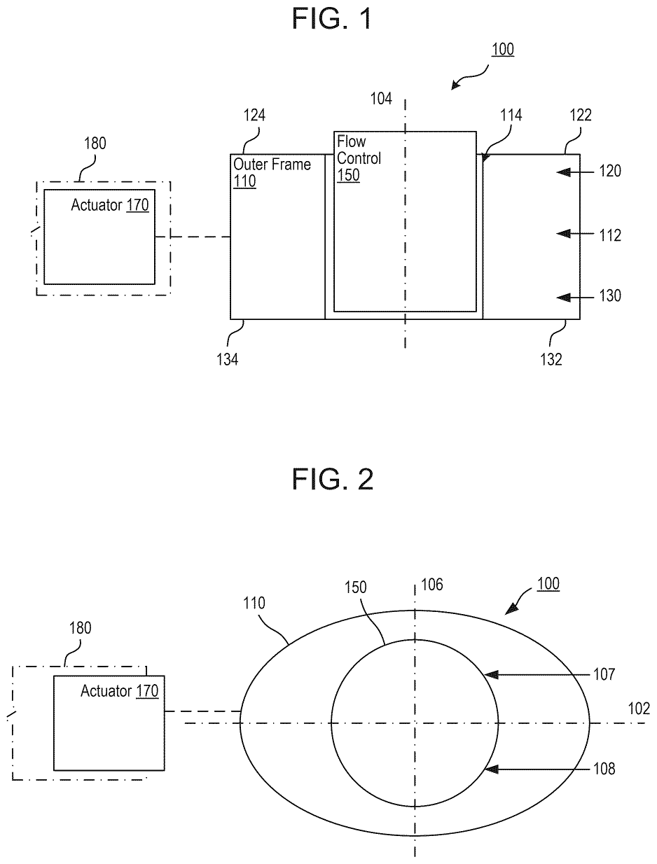

FIGS. 1-5 are various schematic illustrations of a side-deliverable transcatheter prosthetic heart valve 100 (also referred to herein as “prosthetic valve” or simply “valve”) according to an embodiment. As described in further detail herein, the valve 100 generally includes an annular support frame 110 and a flow control component 150 mounted within the annular support frame 110. In addition, FIGS. 1-5 illustrate at least a portion of a delivery/deployment system 180 that can at least temporarily couple to and/or otherwise engage the valve 100 and/or portions thereof to facilitate the delivery and/or deployment of the valve 100 into a desired location of a body. For example, delivery/deployment system 180 can be used to deliver and deploy the prosthetic valve 100 in an annulus of a native valve of a human heart (e.g., a tricuspid, mitral, aortic, and/or pulmonary valve of the human heart) and once deployed, the prosthetic valve 100 is configured to permit blood flow in a first direction (e.g., through or via the flow control component 150) from an inflow end of the prosthetic valve 100 to an outflow end of the prosthetic valve 100 and to block blood flow in a second direction, opposite the first direction. Thus, the prosthetic valve 100 can be configured to supplement and/or replace the functioning of the native valve. In some embodiments, the valve 100 and/or the delivery/deployment system 180 can be similar to and/or substantially the same as the valve(s) and/or the delivery/deployment system(s) described in WIPO Patent Publication No. WO 2021/040996 (referred to herein as “the '996 PCT”), filed Aug. 6, 2020, entitled “Side-Deliverable Transcatheter Prosthetic Valves and Methods for Delivering and Anchoring the Same” and/or WIPO Patent Publication No. WO 2021/035032 (referred to herein as “the '032 PCT”), filed Aug. 20, 2020, entitled “Delivery and Retrieval Devices and Methods for Side-Deliverable Transcatheter Prosthetic Valves,” the disclosure of each of which is incorporated herein by reference in its entirety.

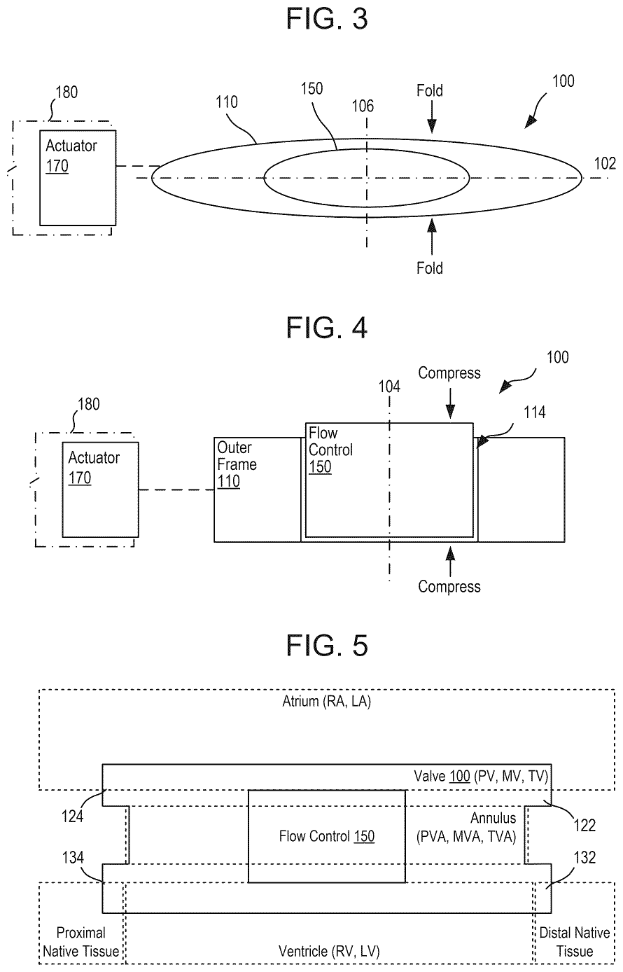

The prosthetic valve 100 is compressible and expandable between an expanded configuration (FIGS. 1 and 2) for implanting at a desired location in a body (e.g., a human heart) and a compressed or delivery configuration (FIGS. 3 and 4) for introduction into the body via, for example, a delivery catheter 182 of the delivery/deployment system 180. The prosthetic valve 100 can be compressible and expandable in at least one direction relative to a longitudinal axis 102 of the valve 100 (also referred to herein as “horizontal axis,” “long-axis,” or “lengthwise axis”). For example, the valve 100 can compressible/expandable along a central axis 104, with a first height or size along the central axis 104 when in the expanded configuration (FIG. 1) and a second height or size, less than the first height or size, along the central axis 104 when in the compressed configuration (FIG. 3). In some embodiments, the prosthetic valve 100 can be compressible and expandable in at least two directions relative to the longitudinal axis 102 of the valve 100. For example, the valve 100 can be compressible/expandable along the central axis 104 (as just described) and compressible/expandable along a lateral axis 106 that is perpendicular to each of the longitudinal axis 102 and the central axis 104 (see e.g., FIGS. 1 and 2). In such embodiments, the valve 100 can have the first height and a first width when in the expanded configuration (FIGS. 1 and 2) and can have a second height and a second width—less than the first height and first width, respectively—when in the compressed configuration (FIGS. 3 and 4).

When in the expanded configuration shown in FIGS. 1, 2, and 5, the valve 100 has an extent in any direction orthogonal or lateral to the longitudinal axis 102 (e.g., along the central axis 104 and/or the lateral axis 106) that is larger than a diameter of the lumen of the delivery catheter 182 used to deliver the valve 100. For example, in some embodiments, the valve 100 can have an expanded height (e.g., along the central axis 104) of 5-60 mm. In some embodiments, the valve 100 can have an expanded length (e.g., along the longitudinal axis 102) and width (e.g., along the lateral axis 106) of about 20-80 mm, or about 40-80 mm. When in the compressed configuration shown in FIGS. 3 and 4, the valve 100 has an extent in any direction orthogonal or lateral to the longitudinal axis 102 (e.g., along the central axis 104 and/or the lateral axis 106) that is smaller than the diameter of the lumen of the delivery catheter 182, allowing the valve 100 to be delivered therethrough. For example, in some embodiments, the valve 100 can have a compressed height (e.g., along the central axis 104) and a compressed width (e.g., along the lateral axis 106) of about 5-15 mm, about 8-12 mm, or about 9-10 mm. The valve 100 can be compressed by compressing, rolling, folding, and/or any other suitable manner, or combinations thereof. In some implementations, the length of the valve 100 (e.g., along the longitudinal axis 102) is not compressed for or during delivery. Rather, in some implementations, the length of the valve 100 can be increased in response to compression of the valve 100 along the central axis 104 and/or the lateral axis 106.

In some embodiments, the valve 100 (and/or at least a portion thereof) may be heat-shaped and/or otherwise formed into any desired shape such as, for example, a roughly tubular shape, a roughly hourglass shape, and/or the like. In some embodiments, the valve 100 can include a supra-annular section or region (e.g., an upper atrial cuff or flange for atrial sealing), a subannular section or region (e.g., a lower ventricle cuff or flange for ventricular sealing), and a transannular section or region (e.g., a body section, a tubular section, a cylindrical section, etc.) disposed therebetween. The transannular region can have an hourglass cross-section for about 60-80% of the circumference to conform to the native annulus along the posterior and anterior annular segments while remaining substantially vertically flat along 20-40% of the annular circumference to conform to the septal annular segment.

While the valve 100 is shown in FIGS. 1-5 as having a generic shape, it should be understood that the size and/or shape of the valve 100 (and/or at least a portion thereof) can be based on a size and/or shape of the anatomical structures of the native tissue. For example, the valve 100 can be centric (e.g., radially symmetrical relative to a central axis 104) or eccentric (e.g., radially asymmetrical relative to the central axis 104). In some eccentric embodiments, the valve 100, or an outer frame thereof, may have a complex shape determined by the anatomical structures where the valve 100 is being mounted. For example, in some instances, the valve 100 may be deployed in an annulus of a native tricuspid valve having a circumference in the shape of a rounded ellipse with a substantially vertical septal wall, which is known to enlarge in disease states along an anterior-posterior line. In some instances, the valve 100 may be deployed in an annulus of a native mitral valve (e.g., near the anterior leaflet) having a circumference in the shape of a rounded ellipse with a substantially vertical septal wall, which is known to enlarge in disease states.

As such, the valve 100 can have a complex shape that is determined, at least in part, by the native annulus and/or a disease state of the native valve. By way of example, the valve 100 or the outer frame thereof may have a D-shape (viewed from the top) so the flat or substantially flat portion can be matched to the anatomy in which the valve 100 will be deployed (e.g., a substantially vertical septal wall). In some embodiments, the valve 100 or the outer frame thereof can have a circumference in the shape of a rounded ellipse, such as a hyperbolic paraboloid, to account for the positions of native septal, anterior, and/or posterior leaflets, and/or the native septal wall; to avoid native electrical bundles such as the atrioventricular (A-V) node and/or A-V node-related structures like the Triangle of Koch, AV bundle, etc.; to avoid interference with coronary blood flow such as the coronary sinus; to accommodate variances in the septal wall that is known to be substantially vertical but that enlarges along the anterior-posterior axis toward the free wall in disease states; and/or the like.

As shown, the valve 100 generally includes an annular support frame 110 and a flow control component 150 mounted within the annular support frame 110. In addition, the valve 100 and/or at least the annular support frame 110 of the valve 100 can include, can couple to, and/or can otherwise engage the delivery/deployment system 180. The annular support frame 110 (also referred to herein as “valve frame,” “wire frame,” “outer frame,” “support frame,” “frame,” etc.) can have a supra-annular region 120, a subannular region 130, and a transannular region 112, disposed and/or coupled therebetween. In some embodiments, the frame 110 can be monolithically and/or unitarily constructed. In some embodiments, the supra-annular region 120, the subannular region 130, and/or the transannular region 112 can be separate, independent, and/or modular components that are coupled to collectively form the frame 110. For example, in some embodiments, the supra-annular region 120 can be an atrial collar, cuff, portion, and/or the like coupled to a top, upper, and/or supra-annular edge of the transannular region 112 and the subannular region 130 can be a ventricular collar, cuff, portion, and/or the like coupled to a bottom, lower, and/or subannular edge of the transannular region 112. Alternatively, the subannular region 130 can be and/or can be formed by a bottom, lower, and/or subannular portion or section of the transannular region 112.

In some implementations, a modular and/or at least partially modular configuration can allow the frame 110 to be adapted to a given size and/or shape of the anatomical structures where the valve 100 is being mounted. For example, the supra-annular region 120, the subannular region 130, and/or the transannular region 112 can be designed and/or adapted so that that the support frame 110 has any desirable height, outer diameter, and/or inner diameter such as any of those described above. Moreover, such a modular configuration can allow the frame 110 to bend, flex, compress, fold, roll, and/or otherwise reconfigure without plastic or permanent deformation thereof. For example, the frame 110 is compressible to a compressed or delivery configuration for delivery and when released it is configured to return to its original shape (uncompressed, expanded, or released configuration) substantially without plastic or permanent deformation.

The support frame 110 and/or the supra-annular region 120, subannular region 130, and/or transannular region 112 can be formed from or of any suitable material. In some embodiments, the frame 110 and/or one or more portions or regions thereof can be formed from or of a shape-memory or superelastic metal, metal alloy, plastic, and/or the like. For example, the frame 110 (e.g., the supra-annular region 120, the subannular region 130, and/or the transannular region 112) can be formed from or of Nitinol or the like. In some embodiments, the frame 110 (and/or any of the regions thereof) can be laser cut from a Nitinol sheet or tube. In other embodiments, the frame 110 (and/or any of the regions thereof) can be formed of or from a Nitinol wire that is bent, kink, formed, and/or manipulated into a desired shape. In still other embodiments, the frame 110 (and/or any of the regions thereof) can be formed of or from a desired material using any suitable additive or subtractive manufacturing process such as those described above. Moreover, the frame 110 and/or one or more of the supra-annular region 120, the subannular region 130, and the transannular region 112 can be formed of or from a metal or other structural frame material, which in turn, is covered by a biocompatible material such as, for example, pericardium tissue (e.g., Dura-Guard®, Peri-Guard®, Vascu-Guard®, etc.), polymers (e.g., polyester, Dacron®, etc.), and/or the like, as described above.

The supra-annular region 120 of the frame 110 can be and/or can form, for example, a cuff or collar that can be attached or coupled to an upper edge or upper portion of the transannular region 112. When the valve 100 is deployed within a human heart, the supra-annular region 120 can be an atrial collar that is shaped to conform to the native deployment location. In a tricuspid and/or mitral valve replacement, for example, the supra-annular region 120 (e.g., an atrial collar or cuff) can have various portions configured to conform to the native valve and/or a portion of the atrial floor surrounding the tricuspid and/or mitral valve, respectively. In some implementations, the supra-annular region 120 can be deployed on the atrial floor to direct blood from the atrium into the flow control component 150 of the valve 100 and to seal against blood leakage (perivalvular leakage) around the frame 110 (e.g., through the annulus but outside of the flow control component 150).

In some embodiments, the supra-annular region 120 can be and/or can include a wire frame that is laser cut out of any suitable material. In some embodiments, the supra-annular region 120 can be formed from a tube or sheet of a shape-memory or superelastic material such as, for example, Nitinol and, for example, heat-set into a desired shape and/or configuration. In some embodiments, forming the supra-annular region 120 in such a manner can allow the supra-annular region 120 to bend, flex, fold, compress, and/or otherwise reconfigure substantially without plastically deforming and/or without fatigue that may result in failure or breaking of one or more portions thereof. Moreover, the wire frame of the supra-annular region 120 can be covered by any suitable biocompatible material such as any of those described above.

The supra-annular region 120 includes a distal portion and a proximal portion. In some embodiments, the distal portion can be and/or can include a distal supra-annular anchoring element and/or the like that can engage supra-annular native tissue on a distal side of the annulus as the prosthetic valve 100 is seated into the annulus. In some embodiments, the proximal portion can be and/or can include a proximal supra-annular anchoring element and/or the like that can engage supra-annular native tissue on a proximal side of the annulus as the prosthetic valve 100 is seated in the annulus. In some embodiments, the distal portion and/or the distal supra-annular anchoring element can be sized and/or shaped to correspond to a size and/or shape of the distal portion of the atrial floor of the heart in which the prosthetic valve 100 is disposed. Similarly, the proximal portion and/or the proximal supra-annular anchoring element can be sized and/or shaped to correspond to a size and/or shape of a proximal portion of the atrial floor of the heart. In some embodiments, the distal portion (or the distal supra-annular anchoring element) and/or the proximal portion (or the proximal supra-annular anchoring element) can be actuated to transition between two or mor configurations and/or states (e.g., during deployment or the like).

Although not shown in FIGS. 1-5, the supra-annular region 120 can be shaped and/or formed to include any number of features configured to engage native tissue, one or more other portions of the valve 100, one or more portions of the delivery/deployment system 180, and/or the like. For example, in some embodiments, the supra-annular region 120 can include and/or can form an outer portion and an inner portion that is suspended from and/or coupled to the outer portion. In some implementations, the outer portion can be sized and/or shaped to engage native tissue, the inner portion can provide structure for mounting the flow control component 150 to the support frame 110, and one or more coverings, drums, spacers, struts, splines, and/or structures can be disposed therebetween. In some implementations, a portion of the supra-annular region 120 can be at least temporarily coupled to and/or can at least temporarily receive a portion of the delivery/deployment system 180, at least a portion of an actuator and/or control device, at least a portion of a guidewire (or guidewire catheter), and/or the like (as described in further detail herein).

The transannular region 112 of the support frame 110 is coupled to the supra-annular region 120 and extends from the supra-annular region 120 and at least partially through the annulus of the native valve when the prosthetic valve 100 is seated therein. In some embodiments, the transannular region 112 can be coupled to the supra-annular region 120 such that a desired amount of movement and/or flex is allowed therebetween (e.g., welded, bonded, sewn, bound, and/or the like). For example, in some implementations, the transannular region 112 and/or portions thereof can be sewn and/or sutured to the supra-annular region 120 (and/or portions thereof).

The transannular region 112 can be shaped and/or formed into a ring, a cylindrical tube, a conical tube, D-shaped tube, and/or any other suitable annular shape. In some embodiments, the transannular region 112 may have a side profile of a flat-cone shape, an inverted flat-cone shape (narrower at top, wider at bottom), a concave cylinder (walls bent in), a convex cylinder (walls bulging out), an angular hourglass, a curved and/or graduated hourglass, and/or a ring or cylinder having a flared top, flared bottom, or both. In some embodiments, the transannular region 112 can have a shape and/or size that is at least partially based on a size, shape, and/or configuration of the supra-annular region 120 (and/or subannular region 130) and/or the native annulus in which it is configured to be deployed. For example, the transannular region 112 can have an outer circumference surface for engaging native annular tissue that may be tensioned against an inner aspect of the native annulus to provide structural patency to a weakened native annular ring. Moreover, the transannular region 112 can form and/or define an aperture or central channel 114 that extends along the central axis 104 (e.g., the y-axis). The central channel 114 (e.g., a central axial lumen or channel) can be sized and configured to receive the flow control component 150 across at least a portion of a diameter of the central channel 114.

In some embodiments, the transannular region 112 can be and/or can include a wire frame that is laser cut out of any suitable material. For example, the transannular region 112 can be formed from a tube or sheet of a shape-memory or superelastic material such as, for example, Nitinol and, for example, heat-set into a desired shape and/or configuration. Although not shown in FIGS. 1-5, in some embodiments, the transannular region 112 can include and/or can be formed with two laser cut halves that can be formed into a desired shape and/or configuration and coupled together to form the transannular region 112. Moreover, the wire frame of the transannular region 112 can include any number of struts (e.g., wires) that include, form, and/or define a set of compressible wire cells. The compressible wire cells can be, for example, diamond-shaped or eye-shaped cells having an orientation and/or cell geometry substantially orthogonal to the central axis 104 (FIG. 1) to minimize strain along the struts or wire cells when the transannular region 112 is in a vertical compressed configuration, a rolled and compressed configuration, or a folded and compressed configuration. In some embodiments, forming the transannular region 112 in such a manner can allow the transannular region 112 to bend, flex, fold, deform, and/or otherwise reconfigure (substantially without plastic deformation and/or undue fatigue) in response to lateral folding along or in a direction of the lateral axis 106 (FIG. 4) and/or vertical compression along or in a direction of the central axis 104 (FIG. 3), as described in further detail herein.

As described above with reference to the supra-annular region 120, the wire frame of the transannular region 112 can be covered by any suitable biocompatible material such as any of those described above. In some implementations, the wire frame of at least the supra-annular region 120 and transannular region 112 can be flexibly coupled (e.g., sewn or sutured) and then collectively or separately covered in the biocompatible material. Said another way, at least the supra-annular region 120 and the transannular region 112 can be covered with the biocompatible material prior to being coupled or after being coupled. In embodiments in which the wire frames are covered after being coupled, the biocompatible material can facilitate and/or support the coupling therebetween.

The subannular region 130 of the frame 110 can be and/or can form, for example, a cuff or collar along an end of the transannular region 112 opposite the supra-annular region 120. For example, when the valve 100 is deployed within a human heart, the subannular region 130 can be and/or can form a ventricular collar that is shaped to conform to the native deployment location. In a tricuspid and/or mitral valve replacement, for example, the subannular region 130 or collar can have various portions configured to conform to the native valve and/or a portion of the ventricular ceiling surrounding the tricuspid and/or mitral valve, respectively. In some implementations, the subannular region 130 or at least a portion thereof can engage the ventricular ceiling surrounding the native annulus to secure the valve 100 in the native annulus, to stabilize the valve 100 in the annulus, to prevent dislodging of the valve 100, to sandwich or compress the native annulus or adjacent tissue between the supra-annular region 120 and the subannular region 130 (or lower portion of the transannular region 112), and/or to seal against blood leakage (perivalvular leakage and/or regurgitation during systole) around the frame 110.

In some embodiments, the subannular region 130 is a lower or subannular portion of the transannular region 112 (e.g., the transannular region 112 and the subannular region 130 are monolithically and/or unitarily formed). Said another way, a lower or subannular portion of the transannular region 112 can form and/or include the subannular region 130. In other embodiments, the subannular region 130 is a separate and/or independent component that can be attached or coupled to a lower edge or portion of the transannular region 112, as described above with reference to the supra-annular region 120. In such embodiments, for example, the subannular region 130 can be and/or can include a wire frame that is laser cut out of any suitable material such as a shape-memory or superelastic material like Nitinol, heat-set into a desired shape and/or configuration, covered by any suitable biocompatible material, and attached to a lower edge of the transannular region 112, as described above with reference to the supra-annular region 120. In some implementations, forming the subannular region 130 in such a manner can allow the subannular region 130 to bend, flex, fold, compress, and/or otherwise reconfigure substantially without plastically deforming and/or without undue or undesirable fatigue that may result in failure or breaking of one or more portions thereof.

The subannular region 130 of the frame 110 can be shaped and/or formed to include any number of features configured to engage native tissue, one or more other portions of the valve 100, one or more portions of the delivery/deployment system 180, one or more portions of an actuator and/or control device 170 (or one or more actuators/control devices), and/or the like. For example, as shown in FIG. 1, the subannular region 130 can include and/or can form a distal portion having a distal anchoring element 132 and a proximal portion having a proximal anchoring element 134. In some embodiments, the anchoring elements 132 and 134 are integrally and/or monolithically formed with the subannular region 130 and/or the lower or subannular portion of the transannular region 112.

The distal anchoring element 132 is configured to engage a desired portion of the native tissue on a distal side of the native annulus to facilitate the seating, mounting, and/or deploying of the valve 100 in the annulus of the native valve. The distal anchoring element 132 can extend from the distal portion of the subannular region 130 (or lower portion of the transannular region 112) by about 10-40 mm. In some implementations, the distal anchoring element 132 can be a projection or protrusion extending from the frame 110 (e.g., the subannular region 130 and/or the lower portion of the transannular region 112) and into a distal subannular position relative to the annulus (e.g., the RVOT for tricuspid valve replacement, and/or the like). In such implementations, the distal anchoring element 132 can be shaped and/or biased such that the distal anchoring element 132 exerts a force on the subannular tissue operable to at least partially secure, stabilize, and/or anchor the distal end portion of the valve 100 in the native annulus.

The distal anchoring element 132 optionally can include a guidewire coupler 133 configured to selectively engage and/or receive a portion of a guidewire or a portion of a guidewire catheter. The guidewire coupler 133 is configured to allow a portion of the guidewire or guidewire catheter to extend through an aperture of the guidewire coupler 133, thereby allowing the valve 100 to be advanced over or along the guidewire and/or guidewire catheter during delivery and deployment.

The proximal anchoring element 134 is configured to engage subannular tissue on a proximal side of the native annulus to facilitate the deploying, seating, mounting, and/or securing of the valve 100 in the annulus. The proximal anchoring element 134 can extend from the proximal portion of the subannular region 130 (or lower portion of the transannular region 112) by about 10-40 mm.

In some embodiments, the proximal anchoring element 134 can be configured to transition, move, and/or otherwise reconfigure between two or more configurations. For example, the proximal anchoring element 134 can be transitioned between a first configuration in which the proximal anchoring element 134 extends from the subannular region 130 a first amount or distance and a second configuration in which the proximal anchoring element 134 extends from the subannular region 130 a second amount or distance, different from the first amount or distance. In some implementations, the proximal anchoring element 134 in the expanded or deployed configuration (e.g., the second configuration) can extend from the transannular region 112 by about 10-40 mm and in the compressed or undeployed configuration (e.g., the first configuration) can be in contact with the transannular region 112 or can extend from the transannular region 112 by less than about 10 mm. In some embodiments, the proximal anchoring element 134 can have a first configuration in which the proximal anchoring element 134 is in a compressed, cinched, contracted, retracted, undeployed, folded, and/or restrained state (e.g., in a position that is near, adjacent to, and/or in contact with the transannular region 112 and/or the supra-annular region 120 of the frame 110), and a second configuration in which the proximal anchoring element 134 is in an expanded, extended, deployed, uncinched, unfolded, and/or unrestrained state (e.g., extending away from the transannular region 112). In some embodiments, the proximal anchoring element 134 can have a first or cinched configuration in which the proximal anchoring element 134 is folded, flipped, or pulled under the prosthetic valve 100 and a second or uncinched configuration in which the proximal anchoring element 134 extends away from the prosthetic valve 100 (e.g., increases a perimeter of the subannular region 130). In some embodiments, the proximal anchoring element 134 can have a first or cinched configuration in which the proximal anchoring element 134 is folded, flipped, or pulled along a side of the valve or transannular region 112 (e.g., a septal side or a freewall side).

In some implementations, at least a portion of the transannular region 112 can be at least partially reconfigured based on the state and/or configuration of the proximal anchoring element 134. For example, placing the proximal anchoring element 134 in a compressed state or configuration can also at least partially compress or reconfigure at least a proximal portion of the transannular region 112. Moreover, in some implementations, the proximal anchoring element 134 can be transitioned from the first configuration to the second configuration in response to actuation of the actuator and/or control device 170, and/or other suitable portion of the delivery/deployment system 180, and/or the like, as described in further detail herein.

In some implementations, the proximal anchoring element 134 can be transitioned from the first configuration to the second configuration during deployment to selectively engage native tissue, chordae, trabeculae, annular tissue, leaflet tissue, and/or any other anatomic structures to aid in the securement of the valve 100 in the native annulus. The proximal anchoring element 134 (and/or the distal anchoring element 132 and/or any other portion of the subannular region 130) can include any suitable feature, surface, member, etc. configured to facilitate the engagement between the proximal anchoring element 134 (and/or the distal anchoring element 132 and/or any other portion of the subannular region 130) and the native tissue. For example, in some embodiments, the proximal anchoring element 134 can include one or more features (e.g., bumps, protrusions, ridges, waves, beads, fingers, hooks, etc.) configured to engage and/or become entangled in the native tissue, chordae, trabeculae, annular tissue, leaflet tissue, and/or any other anatomic structures when in the second configuration.

While the proximal anchoring element 134 is described above as being configured to transition between two or more configurations, in other embodiments, the proximal anchoring element 134 can be and/or can have a substantially fixed configuration. In such embodiments, the proximal anchoring element 134 can be flexible and/or movable through a relatively limited range of motion but otherwise has a single, fixed configuration.

Although not shown in FIGS. 1-5, the subannular region 130 can include and/or form any number of additional anchoring elements such as, for example, a septal anchoring element and/or the like. For example, the subannular region 130 can include a posterior-septal (PS) tab or anchoring element that can engage posterior-septal tissue to help stabilize the valve in the annulus of the native valve. In some embodiments, a septal subannular anchoring element can be included and configured to engage subannular septal tissue, septal leaflet tissue, and/or any other suitable tissue at, near, and/or along the septum of the heart. In some implementations, when the valve 100 is at least partially inserted into the annulus, the septal anchoring element can extend down the septal wall to pin the native septal leaflet away from, for example, the coapting leaflets of the prosthetic valve 100 and/or to stabilize the valve against any intra-annular rolling forces and/or any intra-annular twisting forces that might affect a desired location or positioning of the prosthetic valve within the annulus, (e.g., tilted, angled, twisted, rolled, etc.).

In some embodiments, anchoring elements included in or extending from the subannular region 130 can be configured with a predetermined atrial or ventricular bias, which in some implementations, may be designed, selected, and/or tuned to allow the subannular anchoring elements to engage the native ventricular tissue with a desired amount of force. For example, in some embodiments, the distal subannular anchoring element 132 may have a slight atrial bias meaning the anchoring element 132 is disposed at or extends at an angle in a supra-annular direction (e.g., toward the annulus). In other embodiments, the distal subannular anchoring element 132 may have a slight ventricular bias meaning the anchoring element 132 is disposed at or extends at an angle in a subannular direction (e.g., away from the annulus). In still other embodiments, the distal subannular anchoring element 132 may have a neutral bias meaning the anchoring element 132 is not disposed at angle and/or otherwise extends in a substantially straight or neutral manner. Similarly, any other subannular anchoring element may have an atrial, ventricular, or neutral bias that can be designed, selected, and/or tuned to allow the anchoring element(s) to engage the native ventricular tissue with a desired amount of force.

Although not shown in FIGS. 1-5, the frame 110 may also have and/or form additional functional elements (e.g., loops, anchors, attachment points, etc.) for attaching accessory components such as biocompatible covers, tissue anchors, releasable deployment/retrieval controls (e.g., the actuator and/or control device 170, a tensile member, a torque cable, a hypotube, portions of the delivery/deployment system 180, support members or tethers, and/or other suitable guides, knobs, attachments, rigging, etc.), and so forth.

The flow control component 150 can refer in a non-limiting sense to a device for controlling fluid flow therethrough. In some embodiments, the flow control component 150 can be a leaflet structure having two, three, four, or more leaflets, made of flexible biocompatible material such a treated or untreated pericardium. The leaflets can be sewn or joined to a support structure such as an inner frame, which in turn, can be sewn or joined to the outer frame 110. The leaflets can be configured to move between an open and a closed or substantially sealed state to allow blood to flow through the flow control component 150 in a first direction through an inflow end of the valve 100 and block blood flow in a second direction, opposite to the first direction, through an outflow end of the valve 100. For example, the flow control component 150 can be configured such that the valve 100 functions, for example, as a heart valve, such as a tricuspid valve, mitral valve, aortic valve, or pulmonary valve, which can open to blood flowing during diastole from atrium to ventricle, and that can close from systolic ventricular pressure applied to the outer surface.

The inner frame and/or portions or aspects thereof can be similar in at least form and/or function to the outer frame 110 and/or portions or aspects thereof. For example, the inner frame can be a laser cut frame formed from or of a shape-memory material such as Nitinol. Moreover, the inner frame can be compressible for delivery and configured to return to its original (uncompressed) shape when released (e.g., after delivery). In some embodiments, the inner frame can include multiple portions or parts that are coupled together to collectively form the inner frame. Such an arrangement can allow the inner frame to transition between a compressed and uncompressed state without undue or undesirable plastic deformation, fatigue, and/or the like. In some embodiments, the inner frame can include and/or can form any suitable number of compressible, elastically deformable diamond-shaped or eye-shaped wire cells, and/or the like. The wire cells can have an orientation and cell geometry substantially orthogonal to an axis of the flow control component 150 to minimize wire cell strain when the inner frame is in a compressed configuration.

In some embodiments, the flow control component 150 and/or the inner frame thereof can have a substantially cylindrical or tubular shape when the valve 100 is in the expanded configuration (see e.g., FIG. 2) and can be configured to elastically deform when the valve 100 is placed in the compressed configuration (see e.g., FIGS. 3 and 4). Although not shown in FIGS. 1-5, in some embodiments, the inner frame of the flow control component 150 can include and/or can be formed with two halves that can be coupled together to allow the inner frame to elastically deform in response to lateral compression or folding along or in a direction of the lateral axis 106 (FIG. 3), as described in further detail herein.

As shown in FIGS. 1-5, the flow control component 150 is mounted within the central channel 114 of the frame 110. More specifically, the flow control component 150 is mounted and/or coupled to the supra-annular region 120 (e.g., an inner portion thereof) and is configured to extend into and/or through the central channel 114 formed and/or defined by the transannular region 112. In some embodiments, the flow control component 150 can be coupled to the supra-annular region 120 via tissue, a biocompatible mesh, one or more woven or knitted fabrics, one or more superelastic or shape-memory alloy structures, which is sewn, sutured, and/or otherwise secured to a portion of the supra-annular region 120. In some embodiments, the flow control component 150 can be coupled to the supra-annular region 120 such that a portion of the flow control component 150 is disposed above and/or otherwise extends beyond the supra-annular region 120 (e.g., extends away from the annulus in the direction of the atrium). In some embodiments, the portion of the flow control component 150 extending above and/or beyond the supra-annular region 120 can form a ridge, ledge, wall, step-up, and/or the like. In some implementations, such an arrangement can facilitate ingrowth of native tissue over the supra-annular region 120 without occluding the flow control component 150.

The flow control component 150 can be at least partially disposed in the central channel 114 such that the axis of the flow control component 150 that extends in the direction of blood flow through the flow control component 150 is substantially parallel to the central axis 104 of the frame 110. In some embodiments, the arrangement of the support frame 110 can be such that the flow control component 150 is centered within the central channel 114. In other embodiments, the arrangement of the support frame 110 can be such that the flow control component 150 is off centered within the central channel 114. In some embodiments, the central channel 114 can have a diameter and/or perimeter that is larger than a diameter and/or perimeter of the flow control component 150. Although not shown in FIGS. 1-5, in some embodiments, the valve 100 can include a spacer or the like that can be disposed within the central channel 114 adjacent to the flow control component 150. In other embodiments, a spacer can be a cover, or the like coupled to a portion of the frame 110 and configured to cover a portion of the central channel 114. In some instances, the spacer can be used to facilitate the coupling of the flow control component 150 to the frame 110.

Referring back to FIG. 1, the valve 100 includes and/or is coupled to the actuator and/or control device 170 and the delivery/deployment system 180 (and/or an interface or portion thereof). The actuator and/or control device 170 (also referred to herein as “actuator” or “control device,” interchangeably) can be any suitable member, mechanism, and/or device configured to actuate at least a portion of the valve 100. For example, in some embodiments, the actuator 170 and/or a portion of the actuator 170 can be configured to at least temporarily couple to the supra-annular region 120 of the support frame 110 (e.g., a spline and/or other portion thereof) and can be configured to actuate one or more portions of the valve 100. More specifically, the actuator 170 can be configured to actuate one or more portions of the valve 100 such as, for example, at least the proximal anchoring element 134 of the subannular region 130 of the support frame 110 to transition the proximal anchoring element 134 between its first and second configurations. In some implementations, the actuator 170 can include one or more cables, tethers, linkages, joints, connections etc., that can exert a force (or can remove an exerted force) on a portion of the proximal anchoring element 134 operable to transition the proximal anchoring element 134 between the first and second configuration. For example, the subannular region 130 of the support frame 110 can be formed with the proximal anchoring element 134 biased in the uncompressed and/or expanded configuration and the actuator 170 can be actuated to exert a force, via the one or more cables, tethers, etc., operable to transition the proximal anchoring element 134 to the compressed and/or retracted configuration.

As described above, in some implementations, the proximal anchoring element 134 can be in the first configuration for delivery and deployment prior to seating the valve 100 in the native annulus. Once the valve 100 is seated in the native annulus, a user can manipulate a portion of the delivery system to actuate the actuator 170. In this example, actuating the actuator 170 can cause the actuator 170 to release and/or remove the force exerted on the proximal anchoring element 134 (e.g., via the cable(s), tether(s), etc.), thereby allowing the proximal anchoring element 134 to return to its original or biased configuration (e.g., a second configuration), as described above.

The delivery/deployment system 180, shown in FIG. 1, can include any number of components having any suitable shape, size, and/or configuration. In some implementations, the delivery/deployment system 180 can be and/or can include, for example, a distal end portion used to position and/or deliver the valve 100 to a desired location in the body of a patient (e.g., the annulus of a native heart valve). In some embodiments, the delivery/deployment system 180 can include a delivery catheter such as, for example, a 22-34 Fr delivery catheter with any suitable corresponding internal lumen diameter sufficient to receive the prosthetic valve 100 in the compressed or delivery configuration. Moreover, the delivery/deployment system 180 can include a secondary catheter (also referred to as a control catheter) that can be, for example, a multi-lumen catheter configured to engage the valve 100 to advance the valve 100 through the delivery catheter. In some embodiments, each lumen of the multi-lumen secondary catheter can include and/or can receive, for example, a cable, tether, and/or any other suitable component associated with and/or included in the actuator 170. Each cable, tether, and/or component can, in turn, be coupled to a portion of the valve 100 or support frame 110 and configured to actuate a portion thereof, as described in further detail herein with reference to specific embodiments.

Furthermore, a lumen of the multi-lumen secondary catheter (e.g., a central lumen) can include and/or can receive one or more components configured to engage the subannular region 130 of the valve 100 such as, for example, a torque cable, a guidewire, and/or a guidewire catheter. For example, a guidewire and/or guidewire catheter (with a guidewire disposed therein) can extend though the central lumen of the secondary catheter and into a desired position relative to the native tissue (e.g., the RVOT) to provide a path along which the valve 100 travels during delivery and/or deployment. As another example, a torque cable can extend through the central lumen of the secondary catheter and can selectively engage one or more portions of the valve 100. A torque cable can be any suitable cable, or the like configured to removably couple to the supra-annular region 120 of the frame 110 (e.g., a waypoint coupled to and/or formed by the supra-annular region 120). In some embodiments, the torque cable is a relatively stiff cable that facilitates delivery and/or deployment of the valve 100 as well as retraction of the valve 100 if desirable. In this manner, the delivery/deployment system 180 or an interface thereof shown in FIG. 1, can include any of the components described above that can be used in and/or otherwise can facilitate the delivery of the valve 100, deployment and/or actuation of the valve 100 or a portion thereof (e.g., the proximal anchoring element 134), and/or retraction of the valve 100. Moreover, the delivery/deployment system 180 (or interface thereof) can be configured to decouple, disengage, and/or otherwise release the valve 100 after the valve 100 is deployed in a native annulus, as described in further detail herein with reference to specific embodiments.

As described above, the valve 100 is compressible and expandable between the expanded configuration and the compressed configuration. The valve 100 can have a first height or size along the central axis 104 when in the expanded configuration and can have a second height or size, less than the first height or size, along the central axis 104 when in the compressed configuration. The valve 100 can also be compressed in additional directions. For example, the valve 100 can be compressed along the lateral axis 106 that is perpendicular to each of the longitudinal axis 102 and the central axis 104 (see e.g., FIGS. 2 and 3). With the valve 100 in the compressed configuration, the delivery system (or at least a portion thereof) can be manipulated to advance the valve 100 into the heart.

FIG. 5 shows the valve 100 seated in an annulus of a native heart valve after delivery and deployment. As described above, the prosthetic valve 100 can be a replacement prosthetic valve for any of the native valves of the human heart-the pulmonary valve, mitral valve, aortic valve, and/or tricuspid valve (PV, MV, AV, TV). More specifically, the valve 100 is configured for transcatheter, orthogonal/side delivery through the delivery catheter 182 to the desired location in the body. During delivery through the delivery catheter 182, the valve 100 is compressed in an orthogonal and/or lateral direction relative to the dimensions of the valve 100 in the expanded configuration (e.g., along the central axis 104 and/or the lateral axis 106, as described above) and the longitudinal axis 102 of the valve 100 is substantially parallel to a longitudinal axis of the delivery catheter 182. In some implementations, for example, the valve 100 (e.g., the supra-annular region 120) can be removably coupled to a control device 170 included in the delivery/deployment system 180 that can be used to advance the compressed valve 100 through a lumen of the delivery catheter 182, and into the atrium (RA, LA) of the heart, as described in detail with reference to the delivery/deployment systems in the '032 PCT.

For example, a distal end portion of the control device 170 can include and/or can be coupled to a connection member or the like that is removably coupled to and in contact with a portion of the valve 100 (e.g., the supra-annular region 120), while a proximal end portion of the control device 170 is proximal to and outside of the delivery catheter 182. Such an arrangement can allow a distally directed force exerted on or at the proximal end portion of the control device 170 to advance the valve 100 along or over a guidewire and/or guidewire catheter (e.g., disposed within and/or extending through the guidewire coupler 133), through the delivery catheter 182, and into the annulus of a native heart valve. Once in the atrium and released from the delivery catheter 182, the valve 100 can transition to the expanded configuration for deployment into an annulus of a native valve such as, for example, the pulmonary valve, the mitral valve, the aortic valve, and/or the tricuspid valve. In some embodiments, at least portion of the control device 170 or the like can extend through one or more lumens of the delivery catheter 182 to a position that is distal to the delivery catheter 182 and within the atrium, thereby allowing a user (e.g., a doctor, surgeon, technician, etc.) to manipulate a distal end of the control device 170 and thus one or more portions of the valve 100 for deployment into the annulus.

Deployment and/or seating of the valve 100 can include, for example, placing the distal anchoring element 132 of the subannular region 130 in a ventricle of the heart (the right ventricle (RV) or the left ventricle (LV)) below the annulus while the remaining portions of the valve 100 are in the atrium (RA, LA). In some instances, the distal anchoring element 132 can be advanced over and/or along the guidewire or guidewire catheter (not shown) to a desired position within the ventricle such as, for example, an outflow tract of the ventricle. For example, in some implementations, the valve 100 can be delivered to the annulus of the native tricuspid valve and at least a portion of the distal anchoring element 132 can be positioned in the RVOT. In other implementations, the valve 100 can be delivered to the annulus of the native mitral valve and at least a portion of the distal anchoring element 132 can be positioned in a subannular position distal to the annulus and/or in any other suitable position in which the distal anchoring element 132 can engage native tissue, leaflets, chordae, etc. In some implementations, a distal portion or surface of the valve 100 can be placed in contact with and/or adjacent to a distal surface of the annular tissue when the distal anchoring element 132 is positioned in the ventricle (e.g., in the RVOT). With the distal portion of the valve 100 in a desired position, the control device 170 can be manipulated to actuate one or more portions of the valve 100 such as, for example, the proximal anchoring element 134 between its first and second configurations. For example, the control device 170 can include one or more cables, tethers, linkages, joints, connections, tensile members, etc., that can exert a force (or can remove an exerted force) on a portion of the proximal anchoring element 134 operable to transition the proximal anchoring element 134 between the first and second configuration. For example, the control device 170 can be actuated to exert a force, via the one or more cables, tethers, etc., to transition the proximal anchoring element 134 to the compressed and/or retracted configuration and can be actuated and/or otherwise manipulated to release or reduce the force to transition-or to allow the transitioning of-the proximal anchoring element 134 from the compressed and/or retracted configuration to the expanded or uncompressed configuration.

With the subannular region 130 of the valve (or valve frame 110) in a desired position/configuration, the control device 170 can be manipulated to push or pivot the proximal portion of the valve 100 into the annulus, thereby seating the prosthetic valve 100. For example, the control device 170 can be and/or can include a steerable control catheter that can be manipulated (steered) to exert a force on a proximal portion of the valve 100 in a direction toward the annulus, thereby pivoting the valve 100 or at least the proximal portion of the valve 100 toward and/or into the annulus. In some implementations, the prosthetic valve 100 can be temporarily maintained in a partially deployed state. For example, the valve 100 can be partially inserted into the annulus and held at an angle relative to the annulus to allow blood to flow from the atrium to the ventricle partially through the native valve annulus around the valve 100, and partially through the valve 100, which can allow for assessment of the valve function.

FIG. 5 shows the valve 100 (PV, MV, AV, TV) placed and/or seated in an annulus (PVA, MVA, AVA, TVA) of the native valve such that the subannular region 130 (e.g., a ventricular collar) is disposed in a subannular position, the transannular region 112 of the valve frame 110 extends through the annulus, and the supra-annular region 120 (e.g., an atrial collar) remains in a supra-annular position. Once the valve 100 is seated in the native annulus (PVA, MVA, AVA, TVA), a user can manipulate a portion of the delivery/deployment system 180 to actuate the control device 170, thereby causing the control device 170 to release and/or remove the force exerted on the proximal anchoring element 134 (e.g., via the cable(s), tether(s), etc.). In turn, the proximal anchoring element 134 can return to its original or biased configuration (e.g., a second configuration).

As described above, supra-annular region 120 of the valve frame 110 (e.g., the atrial cuff) can be configured to engage native atrial tissue, the distal anchoring element 132 can be configured to engage native ventricular tissue on a distal side of the annulus, and the proximal anchoring element 134 can be configured to engage native ventricular tissue on a proximal side of the annulus (e.g., when in the second or expanded configuration), thereby securely seating the valve 100 in the native annulus, as shown in FIG. 5. In some implementations, any other or additional portions of the valve 100 can similarly engage native tissue to securely seat the valve 100 in the native annulus and/or to form a seal between the support frame 110 and the tissue forming the native annulus (e.g., an anterior anchoring element can engage subannular tissue on an anterior side of the annulus, or the supra-annular region 120 can include any number of supra-annular anchoring elements for engaging supra-annular tissue (not shown in FIGS. 1-5)).

With the valve 100 secured in the annulus, the delivery/deployment system 180 (including the control device 170, the guidewire and/or guidewire catheter, and/or any other portion or component of the delivery/deployment system 180) can be decoupled from the valve 100 and retracted/removed from the patient, leaving the prosthetic valve 100 in place. As described above, in some implementations, the arrangement of the actuator 170 can be such that the distal end is wrapped or looped around the guidewire and/or guidewire catheter. In such implementations, withdrawing the guidewire and guidewire catheter into the delivery/deployment system 180 (e.g., proximal to the valve 100) can release the distal end of the actuator 170, thereby allowing the actuator 170 to be retracted and/or withdrawn from the valve 100 and into or through the delivery/deployment system 180. In other implementations, the distal end of the actuator 170 can be decoupled from the attachment points in any suitable manner.

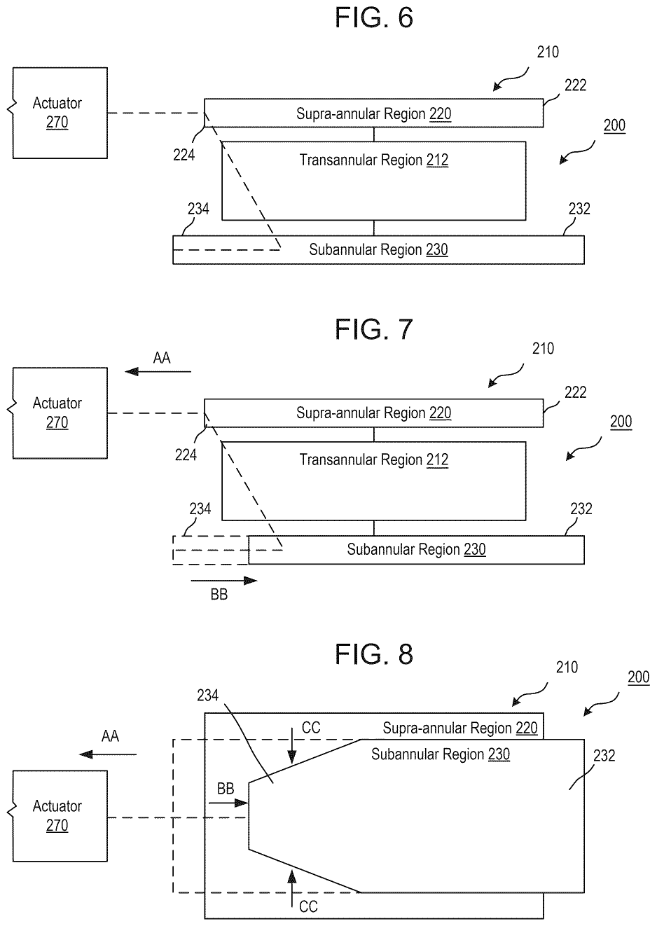

FIGS. 6-8 are schematic illustrations of a prosthetic valve 200 and/or at least an annular support frame 210 thereof removably coupled to an actuator and/or control device 270 according to an embodiment. In some embodiments, the support frame 210 and/or the actuator 270 can be substantially similar in at least form and/or function to the support frame 110 and/or the actuator 170, respectively, described above with reference to FIGS. 1-5. Thus, portions and/or aspects of the support frame 210 and/or the actuator 270 are not described in further detail herein.

As shown, the annular support frame 210 (also referred to herein as “valve frame,” “wire frame,” “outer frame,” “support frame,” or “frame”) can include and/or can have a supra-annular member 220 (or region), a subannular member 230 (or region), and a transannular member 212 (or region), disposed and/or coupled therebetween. In the embodiment shown in FIGS. 6-8, the supra-annular member 220, the subannular member 230, and the transannular member 212 are separate, independent, and/or modular components that are coupled to collectively form the frame 210. Each of the supra-annular member 220, the subannular member 230, and the transannular member 212 are a wire frame that is laser cut out of any suitable material such as a shape-memory or superelastic material like Nitinol. In some implementations, each of the supra-annular member 220, the subannular member 230, and the transannular member 212 can be laser cut from a sheet of Nitinol and, for example, heat-set into a desired shape and/or configuration. As described above, forming the supra-annular member 220, the subannular member 230, and the transannular member 212 in such a manner can provide a desired amount of flexibility and/or resistance to plastic or permanent deformation that can allow the frame 210 to be folded and/or compressed for delivery. Moreover, the wire frame portions of the supra-annular member 220, the subannular member 230, and the transannular member 212 can be covered by any suitable biocompatible material such as any of those described above.

In some embodiments, the supra-annular member 220 of the frame 210 can be similar in at least form and/or function to the supra-annular region 120 (or member) described above with reference to FIGS. 1-5. For example, the supra-annular member 220 can be and/or can form, for example, a cuff or collar that can be attached or coupled to an upper edge or upper portion of the transannular member 212, as described in further detail herein. In some implementations, the supra-annular member 220 can be deployed on the atrial floor to direct blood from the atrium into a flow control component mounted to the frame 210, as described in detail above. The supra-annular member 220 can be shaped and/or formed to include any number of features configured to engage native tissue and/or one or more other portions of the frame 210 and/or the actuator/control device 270. For example, in some embodiments, the supra-annular member 220 can include and/or can form an outer portion or loop, an inner portion or loop, and one or more splines disposed between the outer and inner portions or loops.

In some embodiments, the outer portion or loop (referred to herein as “outer loop”) can be shaped and/or sized to engage native tissue. More specifically, the supra-annular member 220 (or an outer loop thereof) can have a distal portion 222 configured to engage distal supra-annular tissue and a proximal portion 224 configured to engage proximal supra-annular tissue. In some embodiments, the distal and proximal portions 222 and 224 can have a rounded and/or curved shape, wherein a radius of curvature of the proximal portion 224 is larger than a radius of curvature of the distal portion 222. In some implementations, the distal portion 222 can form, for example, a distal upper anchoring element that can engage distal supra-annular tissue to at least partially stabilize and/or secure the frame 210 in the native annulus. Similarly, the proximal portion 224 can form, for example, a proximal upper anchoring element that can engage proximal supra-annular tissue to at least partially stabilize and/or secure the frame 210 in the native annulus.

The inner portion or loop (referred to herein as “inner loop”) of the supra-annular member 220 can be substantially circular and can be coupled to and/or suspended from the outer loop by the one or more splines. As described in further detail herein with reference to specific embodiments, the inner loop can be coupled to an inner frame of the flow control component to at least partially mount the flow control component to the support frame 210. In some implementations, suspending the inner loop from the outer loop (via the one or more splines) can, for example, at least partially isolate the inner loop from at least a portion of the force associated with transitioning the frame 210 between the expanded configuration and the compressed configuration, as described in further detail herein. Moreover, mounting the flow control component to the inner loop of the supra-annular member 220 similarly at least partially isolates and/or reduces an amount of force transferred to the flow control component when the frame 210 is transitioned between its expanded configuration and its compressed configuration.

The one or more splines of the supra-annular member 220 can be any suitable shape, size, and/or configuration. For example, in some embodiments, the supra-annular member 220 can include a distal spline and a proximal spline. As described above, the splines can be configured to support the inner loop and/or otherwise couple the inner loop to the outer loop. In some embodiments, the supra-annular member 220 can include a spline (e.g., a proximal spline) configured to receive, couple to, and/or otherwise engage the actuator/control device 270 and/or delivery/deployment system 180. For example, in some embodiments, a proximal spline can form a connection point, attachment point, waypoint, and/or any other suitable feature that can temporarily and/or removably couple to the actuator/control device 270, as described in further detail herein with reference to specific embodiments.

In some embodiments, the subannular member 230 of the frame 210 can be similar in at least form and/or function to the subannular region 130 described above with reference to FIGS. 1-5. For example, the subannular member 230 of the frame 210 can be and/or can form, for example, a cuff or collar that can be attached or coupled to a lower edge or upper portion of the transannular member 212, as described in further detail herein. When the frame 210 is deployed within a human heart, the subannular member 230 can be a ventricular collar that is shaped to conform to the native deployment location. In a tricuspid and/or mitral valve replacement, for example, the subannular member 230 or collar can have various portions configured to conform to the native valve and/or a portion of the ventricular ceiling surrounding the tricuspid and/or mitral valve, respectively. In some implementations, the subannular member 230 or at least a portion thereof can engage the ventricular ceiling surrounding the native annulus to secure the frame 210 in the native annulus, to prevent dislodging of the frame 210, to sandwich or compress the native annulus or adjacent tissue between the supra-annular member 220 and the subannular member 230, and/or to seal against blood leakage (perivalvular leakage and/or regurgitation during systole) around the frame 210.

The subannular member 230 can be shaped and/or formed to include any number of features configured to engage native tissue, one or more other portions of the frame 210, and/or the actuator 270. For example, in some embodiments, the subannular member 230 can include and/or can form a distal portion having a distal anchoring element 232 and a proximal portion having a proximal anchoring element 234. In some embodiments, the subannular member 230 can include and/or can form any other suitable anchoring element (not shown in FIGS. 6-8). In some embodiments, the anchoring elements 232 and 234 are integrally and/or monolithically formed with the subannular member 230. The distal anchoring element 232 and the proximal anchoring element 234 of the subannular member 230 can be any suitable shape, size, and/or configuration such as any of those described in detail in the '996 PCT and/or the '032 PCT and/or any of those described herein with respect to specific embodiments.

In some embodiments, the distal portion of the subannular member 230 and/or the distal anchoring element 232 can optionally include a guidewire coupler configured to selectively engage and/or receive a portion of a guidewire or a portion of a guidewire assembly. The guidewire coupler is configured to allow a portion of the guidewire or guidewire catheter to extend through an aperture of the guidewire coupler, thereby allowing the frame 210 to be advanced over or along the guidewire during delivery and deployment. In some embodiments, the guidewire coupler and the guidewire or guidewire catheter extending therethrough can be releasably coupled to a portion of the actuator 270 to collectively form a quick release mechanism allowing the portion of the actuator 270 to be decoupled from the valve 200, as described in further detail herein.

The anchoring elements 232 and/or 234 of the subannular member 230 can be configured to engage a desired portion of the native tissue to mount the frame 210 to the annulus of the native valve in which it is deployed. For example, in some implementations, the distal anchoring element 232 can be a projection or protrusion extending from the subannular member 230 and into, for example, a RVOT. In such implementations, the distal anchoring element 232 can be shaped and/or biased such that the distal anchoring element 232 exerts a force on the subannular tissue operable to at least partially secure the distal end portion of the frame 210 in the native annulus. In some implementations, the proximal anchoring element 234 can be configured to engage subannular tissue on a proximal side of the native annulus to aid in the securement of the frame 210 in the annulus.

In some implementations, at least the proximal anchoring element 234 can be configured to transition, move, and/or otherwise reconfigure between a first configuration in which the proximal anchoring element 234 extends from the subannular member 230 a first amount or distance and a second configuration in which the proximal anchoring element 234 extends from the subannular member 230 a second amount or distance. As described above, the subannular member 230 of the frame 210 can be and/or can include, for example, a laser cut frame formed of a shape-memory material such as Nitinol, which is heat-set into a desired shape. In some embodiments, heat-setting the subannular member 230 can include forming one or more twists in a portion of the laser cut wire, which in turn, can allow one or more portions of the subannular member 230 to be biased in different directions and/or orientations. For example, in general, the subannular member 230 of the frame 210 can be formed to provide a high amount of flexibility in a direction that allows the subannular member 230 to be folded and/or compressed (e.g., relative to a longitudinal axis of the subannular member 230). In some embodiments, however, a portion of the subannular member 230 can be twisted and/or otherwise oriented to provide a high amount of flexibility in a direction that allows the proximal anchoring element 234 to be actuated and/or to otherwise transition between its first and second configurations (e.g., in a direction orthogonal to the longitudinal axis of the subannular member 230 and orthogonal to a fold and/or compression direction.

In some embodiments, the proximal anchoring element 234 can be in a compressed, contracted, retracted, undeployed, folded, actuated, cinched, and/or restrained state (e.g., a position that is near, adjacent to, and/or in contact with the transannular member 212 and/or the supra-annular member 220 of the support frame 210) when in the first configuration, and can be in an expanded, extended, deployed, unfolded, unactuated, uncinched, and/or unrestrained state (e.g., extending away from the transannular member 212) when in the second state. In some embodiments, the proximal anchoring element 234 can be biased and/or heat-set in the second configuration. Moreover, in some implementations, the proximal anchoring element 234 can be transitioned in response to actuation of the actuator 270, as described in further detail herein.

In some implementations, the proximal anchoring element 234 can be transitioned from the first configuration to the second configuration during deployment to selectively engage native tissue, chordae, trabeculae, annular tissue, leaflet tissue, and/or any other anatomic structures to aid in the securement of the frame 210 in the native annulus. The proximal anchoring element 234 (and/or the distal anchoring element 232) can include any suitable feature, surface, member, etc. configured to facilitate the engagement between the proximal anchoring element 234 (and/or the distal anchoring element 232) and the native tissue. For example, in some embodiments, the proximal anchoring element 234 can include one or more features configured to engage and/or become entangled in the native tissue, chordae, trabeculae, annular tissue, leaflet tissue, and/or any other anatomic structures when in the second configuration, as described in further detail herein with reference to specific embodiments.

In some embodiments, the transannular member 212 of the frame 210 can be similar in at least form and/or function to the transannular region 112 described above with reference to FIGS. 1-5. For example, the transannular member 212 is disposed between the supra-annular member 220 and the subannular member 230. In some embodiments, the transannular member 212 can be coupled to each of the supra-annular member 220 and the subannular member 230 such that a desired amount of movement and/or flex is allowed therebetween (e.g., welded, bonded, sewn, bound, and/or the like). For example, in some implementations, the transannular member 212 and/or portions thereof can be sewn to each of the supra-annular member 220 and the subannular member 230 (and/or portions thereof). The transannular member 212 can be shaped and/or formed into a ring, a cylindrical tube, a conical tube, D-shaped tube, and/or any other suitable annular shape, as described above with reference to the transannular region 112 (or member). In some embodiments, the transannular member 212 can have a shape and/or size that is at least partially based on a size, shape, and/or configuration of the supra-annular member 220 and/or subannular member 230 of the support frame 210, the flow control component configured to be coupled to the support frame 210, and/or the native annulus in which it is configured to be deployed. For example, the transannular member 212 can have an outer circumference surface for engaging native annular tissue that may be tensioned against an inner aspect of the native annulus to provide structural patency to a weakened native annular ring.

In some embodiments, the transannular member 212 can be and/or can include a wire frame that is laser cut out of any suitable material. For example, the transannular member 212 can be formed from a tube or sheet of a shape-memory or superelastic material such as, for example, Nitinol and, for example, heat-set into a desired shape and/or configuration. Although not shown in FIGS. 6-8, in some embodiments, the transannular member 212 can include and/or can be formed with two laser cut halves that can be formed into a desired shape and/or configuration and coupled together to form the transannular member 212. Moreover, the wire frame of the transannular member 212 can include any number of struts that include and/or define a set of compressible wire cells. The compressible wire cells can be, for example, diamond-shaped or eye-shaped cells having an orientation and/or cell geometry substantially orthogonal to a central axis of the prosthetic valve 200. In some embodiments, forming the transannular member 212 in such a manner can allow the frame 210 to bend, flex, fold, deform, and/or otherwise reconfigure (substantially without plastic deformation and/or undue fatigue) in response to lateral folding along or in a direction of a lateral axis (e.g., extending between the two halves of the transannular member 212) and/or vertical compression along or in a direction of a central axis (e.g., extending along a blood flow direction through the valve 200).

As described above, the supra-annular member 220, the subannular member 230, and the transannular member 212 can be independent and/or modular components that are coupled to collectively form the frame 210. In some embodiments, the supra-annular member 220 is configured to engage supra-annular tissue of the native valve and can be shaped and/or biased to form a substantially fluid tight seal with the atrial floor to limit and/or substantially prevent leakage around the frame (e.g., perivalvular leaks). Similarly, the subannular member 230 is configured to engage subannular tissue of the native valve and can be shaped and/or biased to form a substantially fluid tight seal with the ventricular ceiling to limit and/or substantially prevent leakage around the frame. Moreover, in some implementations, the transannular member 212 can have a slightly oversized circumference relative to the native annular tissue and can, for example, form at least a partial seal between the transannular member 212 of the frame 210 and the native tissue forming the walls of the annulus. In such implementations, forming a seal against the atrial floor, the ventricular ceiling, and the walls of the annulus can provide redundancy in the event of an imperfect or partial seal formed by the supra-annular member 220, the subannular member 230, and/or the transannular member 212.

In some implementations, the distal and proximal anchoring elements 232 and 234 can exert a force on the subannular tissue that is operable in pulling the supra-annular member 220 of the frame 210 toward the atrial floor, thereby facilitating the formation of a seal. In such implementations, for example, the subannular member 230 and/or the transannular member 212 need not form a seal or can form a partial seal with the native tissue because of the seal formed by the supra-annular member 220.

As shown in FIGS. 6-8, the actuator/control device 270 (also referred to herein as “actuator 270” or “control device 270,” interchangeably) can be at least temporarily coupled to the supra-annular member 220 and the subannular member 230. In some embodiments, the actuator 270 or a portion thereof can also at least temporarily couple to a portion of the transannular member 212. The actuator 270 can be any suitable member, mechanism, and/or device configured to actuate at least a portion of the frame 210. Moreover, a portion of the actuator 270 can extend through a portion of a delivery system used to deliver the prosthetic valve 200 including the frame 210. In this manner, a user can manipulate a proximal end portion of the actuator 270 to actuate the actuator 270.

In some embodiments, the actuator 270 and/or a portion of the actuator 270 can be configured to at least temporarily couple to and/or extend through the spline of the supra-annular member 220 (e.g., an attachment point, waypoint, connector, threaded coupler, etc.) and can be configured to actuate one or more portions of the frame 210. The actuator 270 can be configured to actuate at least the proximal anchoring element 234 of the subannular member 230 of the support frame 210 to transition the proximal anchoring element 234 between its first and second configurations (described above).

In some implementations, the actuator/control device 270 can include one or more cables, tethers, linkages, joints, connections etc., that can exert a force (or can remove an exerted force) on a portion of the proximal anchoring element 234 (or more generally, a portion of the subannular member 230) operable to transition at least the proximal anchoring element 234 between the first and second configuration. For example, the actuator 270 can couple to the supra-annular member 220 and can include one or more tethers, cables, and/or members that extend through the waypoint and/or one or more openings or apertures and couple to the proximal anchoring element 234. As described above, the subannular member 230 can be formed with the proximal anchoring element 234 biased in the uncompressed, uncinched, and/or expanded configuration. In this manner, the actuator 270 can be actuated to exert a force, via the one or more cables, tethers, etc., operable to transition the proximal anchoring element 234 to the compressed, cinched, and/or retracted configuration.

More specifically, the user can manipulate the proximal end portion of the actuator 270 to actuate a distal end portion of the actuator 270 that is coupled to the frame 210. For example, actuating the actuator 270 can be such that the one or more cables, tethers, and/or members are pulled in a proximal direction (e.g., away from the frame 210 and/or in a manner that increases a tension therein), as indicated by the arrow AA in FIG. 7. The coupling of the distal end portion of the actuator 270 to the frame 210 can be such that the proximal movement of the cables, tethers, etc., pull at least the proximal anchoring element 234 toward a central axis of the frame 210, as indicated by the arrow BB in FIG. 7. In some implementations, the proximal movement of the cables, tethers, etc. can exert a force of the proximal anchoring element 234 that is operable to fold, flip, or bend the proximal anchoring element 234 under the transannular member 212 (e.g., toward a distal end portion of the valve 200). As such, actuating the actuator 270 can exert a force on the proximal anchoring element 234 operable to place the proximal anchoring element 234 in a compressed, retracted, restrained, cinched, and/or actuated configuration, as shown in FIG. 7.

In some implementations, actuating the actuator 270 also can be operable to pull a proximal-anterior portion of the subannular member 230 and/or transannular wall and a proximal-posterior portion of the subannular member 230 and/or transannular wall to or toward the longitudinal axis of the valve 200. For example, FIG. 8 shows that the actuation of the actuator 270 (e.g., moving the actuator 270 or tethers in the AA direction) compresses and/or moves the proximal anchoring element 234 toward a central portion of the valve frame 210, as indicated by the arrow BB, and compresses the posterior and anterior sidewalls toward a central portion of the valve frame 210, as indicated by the arrows CC. As such, actuating the actuator 270 can reduce a perimeter of at least the subannular member 230 allowing a desired portion of the valve frame 210 to be inserted into the annulus of the native valve.

As described above, the proximal anchoring element 234 can be in and/or can be placed in the first configuration (cinched) for delivery and deployment prior to seating the frame 210 (or valve 200) in the native annulus (e.g., prior to being loaded into the delivery catheter or after being released from the distal end of the delivery catheter). Once the frame 210 is seated in the native annulus, a user can manipulate the proximal portion of the actuator 270 to actuate and/or release the actuator 270. In this example, the actuation can cause the actuator 270 to release and/or remove at least a portion of the force exerted on the proximal anchoring element 234 (e.g., via the cable(s), tether(s), etc.), thereby allowing the proximal anchoring element 234 (and/or one or more portions of the anterior and/or posterior walls) to return to its biased configuration or a second configuration (see e.g., FIG. 6), as described above.

In some embodiments, the actuator 270 can include a first tether that can be placed in tension (e.g., pulled in a proximal direction) to actuate and/or cinch the proximal anchoring element 234 to the first configuration and can include second tether that can be placed in tension (e.g., pulled in a proximal direction) to actuate, uncinch, and/or expand the proximal anchoring element 234 to the second configuration. For example, after seating the prosthetic valve 200 in the native annulus, the first tether can be released and/or actuated to release the tension along the first tether, allowing the proximal anchoring element 234 to transition toward the second configuration. In some instances, however, the proximal anchoring element 234 may contact native tissue or otherwise may be hindered in returning to the second, biased configuration. As such, the actuator 270 can include a second tether or the like that may be actuated and/or placed in tension to pull or otherwise facilitate the transition of the proximal anchoring element 234 from the first configuration to the second configuration, as described in further detail herein.

While the frame 210 and/or one or more portions of the subannular member 230 are described above as being compressed to move inward toward a central axis of the frame 210 in response to actuation of the actuator 270, in other embodiments, the actuator 270 can be removably coupled to one or more portions of the frame 210 and configured to move such portions in any suitable manner. For example, in some implementations, the actuator 270 (e.g., one or more tethers or the like, as described above) can be coupled to the proximal anchoring element 234 such that actuation of the actuator 270 results in the proximal anchoring element 234 folding or wrapping around or under at least a portion of the transannular member 212 of the frame 210 in either an anterior direction or a posterior direction, or both directions depending on the mode of actuation. As described above, the folding and/or wrapping of the proximal anchoring element 234 around or under at least a portion of the transannular member 212 can reduce a circumference or diameter of at least the subannular member 230 allowing the frame 210 to be inserted into and/or at least partially through the annulus of the native heart valve. In some embodiments, the actuator 270 (e.g., cinch tether(s)) travel through the supra-annular member 220 by way of the waypoint and are secured and/or mounted to the transannular member 212 and/or the proximal anchoring element 234 of the subannular member 230 at one or more attachment points. In some embodiments, the cinch tether(s) can be secured along a strut of the wireframe of the transannular member 212 rather than spanning across one or more of the wire cells or otherwise being unsecured between the supra-annular member 220 and the subannular member 230. In some instances, such a configuration can limit and/or can substantially prevent a cinching of the valve 200 in an axial direction (e.g., cinching in a manner that reduces a height of the valve 200).

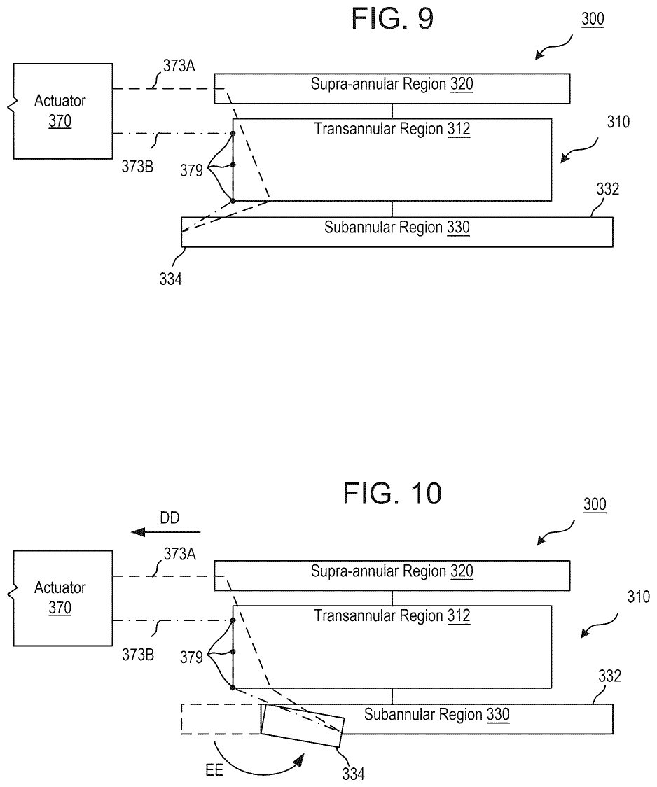

FIGS. 9-12 are schematic illustrations of a prosthetic valve 300 and/or at least an annular support frame 310 thereof removably coupled to an actuator and/or control device 370 according to an embodiment. In some embodiments, the prosthetic valve 300 and/or the actuator/control device 370 can be substantially similar in at least form and/or function to the prosthetic valve 100 and/or 200, and/or the actuator/control device 170 and/or 270, respectively, described above. Thus, portions and/or aspects of the prosthetic valve 300 and/or the actuator/control device 370 may not be described in further detail herein.

The annular support frame 310 (also referred to herein as “valve frame,” “wire frame,” “outer frame,” “support frame,” or “frame”) has a supra-annular member 320 (or region), a subannular member 330 (or region), and a transannular member 312 (or region), disposed and/or coupled therebetween. Each of the supra-annular member 320 (or region), the subannular member 330 (or region), and the transannular member 312 (or region) include a wire frame that is laser cut out of any suitable material such as a shape-memory or superelastic material like Nitinol and at least partially covered by any suitable biocompatible material such as any of those described above. As such, the frame 310 and/or the members/regions thereof can have a desired amount of flexibility and/or resistance to plastic or permanent deformation that can allow the frame 310 to be folded, compressed, actuated, cinched, and/or otherwise reconfigured for delivery and/or deployment into an annulus of a native heart valve.

The supra-annular member 320 of the frame 310 can be and/or can form, for example, a cuff or collar that can be attached or coupled to an upper edge or portion of the transannular member 312. The supra-annular member 320 of the frame 310 can be similar in at least form and/or function to the supra-annular region 120 (or member) and/or the supra-annular member 220 described above and thus, portions and/or aspects of the supra-annular member 320 may be identified for context but may not be described in further detail herein. For example, the supra-annular member 320 can include and/or can form an outer loop configured to selectively contact and/or engage native tissue (e.g., via one or more anchoring elements and/or other features), an inner loop configured to couple the flow control component to the support frame 310, and one or more splines configured to couple or suspend the inner loop from the outer loop. In addition, the one or more splines can be configured to at least temporarily receive, couple to, and/or otherwise engage the actuator 370 and/or a delivery/deployment system (e.g., via one or more connection point(s), attachment point(s), waypoint(s), interfaces, and/or any other suitable feature), as described in further detail herein.

The subannular member 330 of the frame 310 can be and/or can form, for example, a cuff or collar that can be attached or coupled to a lower edge or portion of the transannular member 312, and shaped and/or formed to include any number of features configured to engage native tissue, one or more other portions of the frame 310, and/or the actuator 370. The subannular member 330 can be similar in at least form and/or function to the subannular region 130 (or member) and/or the subannular member 230 described above and thus, portions and/or aspects of the subannular member 330 may be identified for context but may not be described in further detail herein. For example, the subannular member 330 can include and/or can form a distal portion having at least a distal anchoring element 332 and a proximal portion having a proximal anchoring element 334 (e.g., any additional anchoring element(s) not shown in FIGS. 9-12). The anchoring elements 332 and/or 334 of the subannular member 330 can be configured to engage a desired portion of the native tissue to at least partially secure the frame 310 in the annulus of the native valve in which it is deployed. The anchoring elements 332 and 334 can be any suitable shape, size, and/or configuration such as, for example, any of those described above with respect to any of the subannular regions/members described herein with respect to specific embodiments, and/or any of those described in in the '996 PCT and/or the '032 PCT.

For example, the distal anchoring element 332 can be a tab, projection, protrusion, etc. extending from a distal end portion of the subannular member 330 that can be shaped and/or configured to engage and/or exert a force on subannular tissue on a distal side of the native annulus (e.g., tissue of or forming a RVOT) to facilitate deployment, seating, and/or securement of the prosthetic valve 300 in the annulus. In some embodiments, the distal anchoring element 332 can include a guidewire coupler (not shown) configured to selectively engage and/or receive a portion of a guidewire or guidewire catheter, thereby allowing the frame 310 to be advanced over or along the guidewire or guidewire catheter during delivery and deployment. In some embodiments, the guidewire coupler and the guidewire or guidewire catheter extending therethrough can provide and/or can form a structure or feature to which a portion of the actuator 370 can be releasably coupled (e.g., collectively forming a quick release mechanism or the like as described above with reference to the prosthetic valves 100 and/or 200).

The proximal anchoring element 334 of the subannular member 330 can be a tab, projection, protrusion, etc. extending from a proximal end portion of the subannular member 330 that can be shaped and/or configured to engage and/or exert a force on subannular tissue on a proximal side of the native annulus to aid in the securement of the frame 310 in the annulus. In some implementations, at least the proximal anchoring element 334 can be configured to transition, move, and/or otherwise reconfigure (e.g., in response to actuation of one or more portions of the actuator 370) between a first state and/or configuration and a second state and/or configuration. For example, the proximal anchoring element 334 in the first configuration can be in an actuated and/or cinched state and/or the like (e.g., in a position that is near, adjacent to, folded/flipped under or along a side of and/or in contact with the transannular member 312, the flow control component, and/or the like) and the proximal anchoring element 334 in the second configuration can be in an unactuated and/or uncinched state and/or the like (e.g., extending proximally away from the transannular member 312). In some implementations, the proximal anchoring element 334 can be transitioned from the first configuration to the second configuration after the prosthetic valve 300 is seated in the annulus to selectively engage native tissue, chordae, trabeculae, annular tissue, leaflet tissue, and/or any other anatomic structures to aid in the securement of the prosthetic valve 300 in the native annulus. The functions, configurations, and/or features of the proximal anchoring element 334 are described in further detail herein with respect to the relationship between the proximal anchoring element 334 and the actuator 370.

The transannular member 312 of the frame 310 is coupled to and disposed between the supra-annular member 320 and the subannular member 330. The transannular member 312 can be similar in at least form and/or function to the transannular region 112 (or member) and/or the transannular member 212 described above and thus, portions and/or aspects of the transannular member 312 may be identified for context but may not be described in further detail herein. The transannular member 312 can have any suitable shape, size, and/or configuration. For example, the transannular member 312 can have a shape and/or size that is at least partially based on a size, shape, and/or configuration of the supra-annular member 320 and/or the subannular member 330 of the support frame 310, the flow control component, the native annulus in which the prosthetic valve 300 is configured to be deployed, and/or any suitable combination(s) thereof. Moreover, as described above with reference to the transannular region 112 (or member) and/or the transannular member 212, the transannular member 312 can include one or more members that are formed from a shape-memory or superelastic material and that include a set of compressible wire cells having an orientation and/or cell geometry substantially orthogonal to a central axis of the prosthetic valve 200. As such, the transannular member 312 can have a configuration that can allow the frame 210 to bend, flex, fold, deform, and/or otherwise reconfigure (substantially without plastic deformation and/or undue fatigue) in response to lateral folding along or in a direction of a lateral axis and/or vertical compression along or in a direction of the central axis to be placed in, for example, a delivery configuration.

As shown in FIGS. 9-12, the actuator/control device 370 can be included in a delivery/deployment system and is configured to at least temporarily couple to the frame 310. The actuator 370 can be any suitable member, mechanism, and/or device configured to actuate and/or control at least a portion of the frame 310. More specifically, the actuator 370 can be and/or can be a portion of a control device or the like that can couple to the frame 310 and used to deliver the prosthetic valve 300 in the compressed or delivery configuration through a delivery catheter and used to manipulate, control, and/or actuate the prosthetic valve 300 once released from the delivery catheter to deploy the prosthetic valve 300 in the annulus of the native heart valve (e.g., a tricuspid valve or any other valve of the heart). In some embodiments, the actuator 370 and/or a portion of the actuator 370 can be configured to at least temporarily couple to and/or extend through the spline or other portion of the supra-annular member 320 (e.g., an attachment point, waypoint, connector, threaded coupler, etc.) and at least temporarily couple to the proximal anchoring element 334 of the subannular member 330, allowing the actuator 370 to transition the proximal anchoring element 334 between its first and second configurations.

For example, the actuator/control device 370 and/or a portion thereof can be configured to at least temporarily couple to the supra-annular member 320. In some embodiments, the actuator/control device 370 can include a yoke or other connection member that is configured to removably couple to and engage the supra-annular member 320. For example, the actuator/control device 370 can include one or more tethers, sutures, and/or the like configured to removably couple the yoke or other connection member to the supra-annular member 320 (e.g., a drum or other suitable surface or portion of the supra-annular member 320). In addition, the actuator 370 can include one or more cables, tethers, linkages, joints, connections, etc., that can extend through the spline or other portion of the supra-annular member 320 (e.g., an attachment point, waypoint, connector, threaded coupler, etc.) and removably couple to the proximal anchoring element 334 of the subannular member 330. As such, the actuator 370 can be manipulated and/or actuated to transition the proximal anchoring element 334 between its first and second configurations.

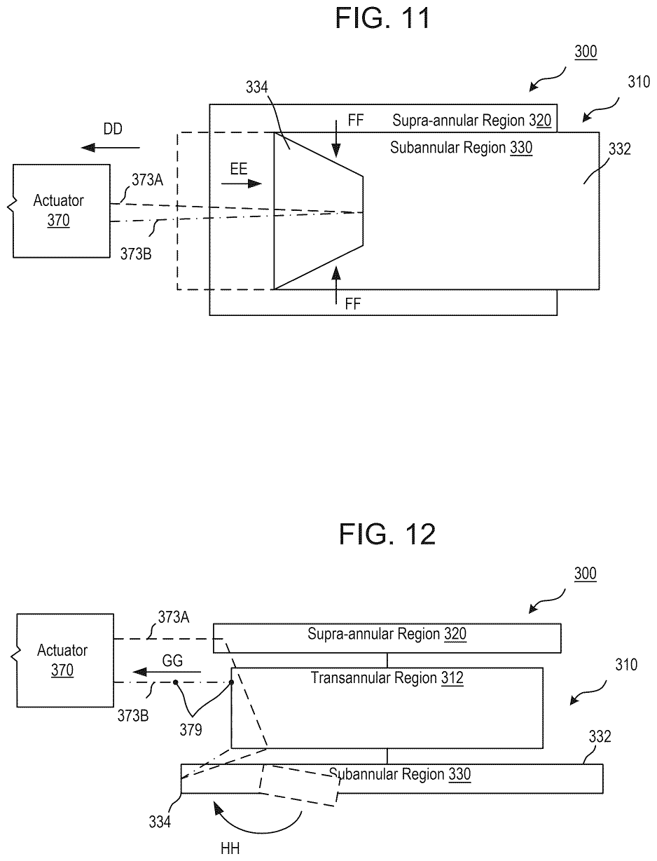

For example, the subannular member 330 can be formed with the proximal anchoring element 334 biased in the uncompressed, expanded, and/or uncinched configuration, as shown in FIG. 9. In this manner, the actuator 370 can be actuated to exert a force, via the one or more cables, tethers, etc., operable to transition the proximal anchoring element 334 to the compressed, retracted, and/or cinched configuration. As shown in FIGS. 9-11, the actuator 370 includes, for example, a first tether 373A and a second tether 373B that are removably coupled to the proximal anchoring element 334. As described above with reference to the actuator 170 and/or 270, a proximal end portion of the first tether 373A can be manipulated and/or actuated, for example, to pull a distal end portion of the first tether 373A in a proximal direction (e.g., away from the frame 310 and/or in a manner that increases a tension therein), as indicated by the arrows DD in FIGS. 10 and 11.

The coupling of the distal end portion of the first tether 373A to the frame 310 is such that the proximal movement of the first tether 373A pulls the proximal anchoring element 334 toward a central axis of the frame 310, placing the proximal anchoring element 334 in the first configuration. For example, as described above with reference to the actuator 270, the first tether 373A can travel through the supra-annular member 320 by way of the waypoint and can be secured, mounted, and/or routed to or through one or more attachment points of the transannular member 312, the subannular member 330, and/or the proximal anchoring element 334. The arrangement of the attachment points can be such that at least a portion of the attachment points allow the first tether 373A to be directed and/or routed in a manner that allows the force exerted on the proximal anchoring element 334 to be oriented in a predetermined and/or desired direction. For example, the first tether 373A can be routed through one or more attachment points (e.g., along the transannular member 312 and/or the subannular member 330) that are distal to the proximal anchoring element 334 such that increasing a tension along the first tether 373A exerts a force on the proximal anchoring element 334 operable to pull, flip, and/or fold the proximal anchoring element 334 in a distal direction and/or otherwise to a distal position (e.g., flipped and/or folded below a portion of the subannular member 330 and/or otherwise cinched backwards (distally) relative to the biased state or position), as indicated by the arrows EE in FIGS. 10 and 11. In addition, the first tether 373A can be routed and/or secured along a strut of the wireframe/wire cells of the transannular member 312 rather than spanning across the wire cells or otherwise being unsecured between the supra-annular member 320 and the subannular member 330, which in turn, can limit and/or substantially prevent cinching or compressing of at least a proximal portion of the valve 300 in a vertical, axial direction, as described above with reference to the prosthetic valve 200. While the proximal anchoring element 334 is shown and described as being flipped and/or folded below the subannular member 330 and/or otherwise cinched backwards, in some embodiments, the proximal anchoring element 334 can be pulled, flipped, and/or folded to a position along a side of the transannular member 312 (e.g., a septal side or a freewall side of the transannular member 312).

As indicated by the arrows FF in FIG. 11, actuating the actuator 370 also can be operable to pull a proximal-anterior portion of the subannular member 330 and/or transannular wall and a proximal-posterior portion of the subannular member 330 and/or transannular wall to or toward a center portion and/or the longitudinal axis of the valve 300. In some implementations, the first tether 373A can be routed in a manner that cinches the proximal anchoring element 334 and cinches, compresses, and/or squeezes the anterior/posterior portions or walls (as indicated by the arrows EE and FF, respectively, in FIGS. 10 and 11). In other implementations, the actuator 370 can include one or more tethers (other than the first tether 373A) that can be actuated and/or pulled to transition the anterior/posterior portions or walls independent of, or at least partially independent of, the actuation of the proximal anchoring element 334.

Actuating the actuator 370 can reduce a perimeter of at least the subannular member 330 allowing a desired portion of the prosthetic valve 300 to be inserted or “dropped” into the annulus of the native valve. For example, the proximal anchoring element 334 can be and/or can be placed in the first configuration prior to seating the prosthetic valve 300 in the native annulus. Once the prosthetic valve 300 is seated in the native annulus, a user can manipulate the proximal portion of the actuator 370 to actuate and/or release at least a portion of the actuator 370. More specifically, the actuator 370 can be actuated to release or reduce an amount of tension along the first tether 373A, thereby reducing or removing the force exerted on the proximal anchoring element 334 operable to maintain the proximal anchoring element 334 in the first configuration. Accordingly, the proximal anchoring element 334 can be allowed to return to or toward its second or biased configuration.

Alternatively, in the embodiment shown in FIGS. 9-12, the actuator 370 includes a second tether 373B that can be actuated or pulled, after the tension along the first tether 373A is released, to aid and/or facilitate the transition of the proximal anchoring element 334 to the second or biased configuration. For example, in some instances, the proximal anchoring element 334 may contact native tissue and/or otherwise may be hindered in returning to the second, biased configuration. As such, the second tether 373B can be actuated to increase a tension along the second tether 373B operable to pull or otherwise facilitate the transition of the proximal anchoring element 334 from the first configuration to the second configuration. As described above with reference to the first tether 373A, the second tether 373B can be directed and/or routed through at least the transannular member 312 and removably coupled to the proximal anchoring element 334 in such a manner that a proximally directed force that increases a tension along the second tether 373B (indicated by the arrow GG in FIG. 12) pulls, flips, and/or unfolds the proximal anchoring element 334 in a proximal direction (indicated by the arrow HH in FIG. 12). For example, in some embodiments, the second tether 373B can extend through an attachment point, opening, waypoint, and/or the like formed by the transannular member 312 allowing at least a portion of the second tether 373B to be disposed outside the prosthetic valve 300, thereby allowing the second tether 373B to pull the proximal anchoring element 334 back to the second or biased configuration.

In some embodiments, the second tether 373B can include one or more locking features that can at least temporarily lock the proximal anchoring element 334 in the second configuration or in one or more other configurations. For example, as shown in FIG. 10, the second tether 373B can include a set of locks 379 along a length of the second tether 373B. The locks 379 can be, for example, knots, beads, bumps, protrusions, and/or any other suitable feature disposed at desired positions along the second tether 373B that correspond to one or more desired positions or configurations of the proximal anchoring element 334. Moreover, the second tether 373B can be routed through one or more locking features of the frame 310 (e.g., attachment points, openings, couplers, reinforced rings, and/or the like of the transannular member 312 and/or supra-annular member 320) that can selectively engage the lock(s) 379 of the second tether 373B. For example, the second tether 373B can be in a distal position or can be pulled in a distal direction when the proximal anchoring element 334 is in the first configuration. In this position or state, the lock(s) 379 can be distal to the locking feature of the frame 310.

In some implementations, however, actuating the second tether 373B to pull or aid in the transition of the proximal anchoring element 334 to the second or biased configuration can pull the second tether in a proximal direction, which in turn, pulls the lock(s) 379 along the second tether 373B past and/or through the locking feature(s) of the frame 310. In some embodiments, the lock(s) 379 along the second tether 373B can be at least partially compressible allowing the lock(s) 379 (e.g., having at least a slightly larger size) through the locking feature(s) (e.g., having at least a slightly smaller size). Thus, once the proximal anchoring element 334 is in the second configuration (or any other suitable configuration such as vertically or axially cinched configuration in a direction of the atrium), the lock(s) 379 can be proximal to the locking feature(s) of the frame 310. Moreover, releasing the tension along the second tether 373B can be such that a portion of the second tether 373B between the proximal anchoring element 334 and the locking feature(s) of the frame 310 is at least partially under tension which can place the lock(s) 379 of the second tether 373B in contact with a proximal side of the locking feature(s) of the frame 310, thereby limiting and/or substantially preventing the proximal anchoring element 334 from moving toward the first or cinched configuration.

Although not shown in FIGS. 9-12, the actuator 370 and/or the first and second tethers 373A and 373B thereof can include and/or can form one or more quick release mechanisms allowing the actuator 370 to be decoupled from the prosthetic valve 300 after the prosthetic valve 300 is seated and secured in the annulus. For example, as described above, the delivery system can include a guidewire and/or guidewire catheter that can extend from the actuator/control device 370, through the waypoint of the supra-annular member 320, below the flow control component, and through a guidewire coupler of the distal anchoring element 332. In some embodiments, at least the first tether 373A can include and/or can form a loop at a distal end thereof, which can be disposed about a portion of the guidewire or guidewire catheter distal to an attachment point along the subannular member 330 and the guidewire coupler. As such, withdrawing the guidewire or guidewire catheter to a position proximal to the attachment point along the subannular member 330 decouples and/or releases the distal end of at least the first tether 373A, thereby allowing the first tether 373A to be retracted into the actuator 370 and/or delivery system.

In some embodiments, the second tether 373B can similarly couple to and/or at least partially form a quick release mechanism. For example, in some embodiments, the second tether 373B can include a first portion that removably or releasably couples to a second portion. The first portion of the second tether 373B can be routed through the delivery system such that a proximal end thereof is proximal to a delivery catheter and a distal end thereof is releasably or removably coupleable to the second portion of the second tether 373B. The second portion of the second tether 373B can be, for example, a distal portion that is coupled to the proximal anchoring element 334 (e.g., releasably or permanently) and that extends a relatively short distance from the proximal anchoring element 334 to releasably or removably couple to the first portion of the second tether 373B. For example, the second portion can include and/or can form one or more loops, couplers, connectors, etc. that can engage and/or removably couple to the first portion of the second tether 373B. Accordingly, the second portion of the second tether 373B can form a releasable connector, loop, suture, tether, etc. allowing the actuator 370 to be decoupled from the prosthetic valve 300. In some embodiments, the releasable connector, loop, suture, tether, etc. can be similar to and/or substantially the same as any of those described in U.S. Pat. No. 12,150,852 (referred to herein as “the '852 patent”), filed Feb. 7, 2022, entitled, “Proximal Tab for Side-Delivered Transcatheter Heart Valves and Method of Delivery,”the disclosure of which is incorporated herein by reference in its entirety.

In some embodiments, the distal anchoring element 332 can include a guidewire coupler (not shown) configured to selectively engage and/or receive a portion of a guidewire or guidewire catheter, thereby allowing the frame 310 to be advanced over or along the guidewire or guidewire catheter during delivery and deployment. In some embodiments, the guidewire coupler and the guidewire or guidewire catheter extending therethrough can provide and/or can form a structure or feature to which a portion of the actuator 370 can be releasably coupled (e.g., collectively forming a quick release mechanism or the like as described above with reference to the prosthetic valves 100 and/or 200).

As described above, the proximal anchoring element 334 is in the first or cinched configuration prior to seating the prosthetic valve 300 in the annulus of the native valve. In some implementations, the process of delivering and deploying the prosthetic valve 300 in the annulus can include transitioning and/or cinching the proximal anchoring element 334 after the prosthetic valve 300 is released from a distal end of the delivery catheter (and prior to seating the prosthetic valve 300 in the annulus). In other implementations, however, the process of delivering and deploying the prosthetic valve 300 can include transitioning and/or cinching the proximal anchoring element 334 prior to loading the prosthetic valve 300 in the delivery system.

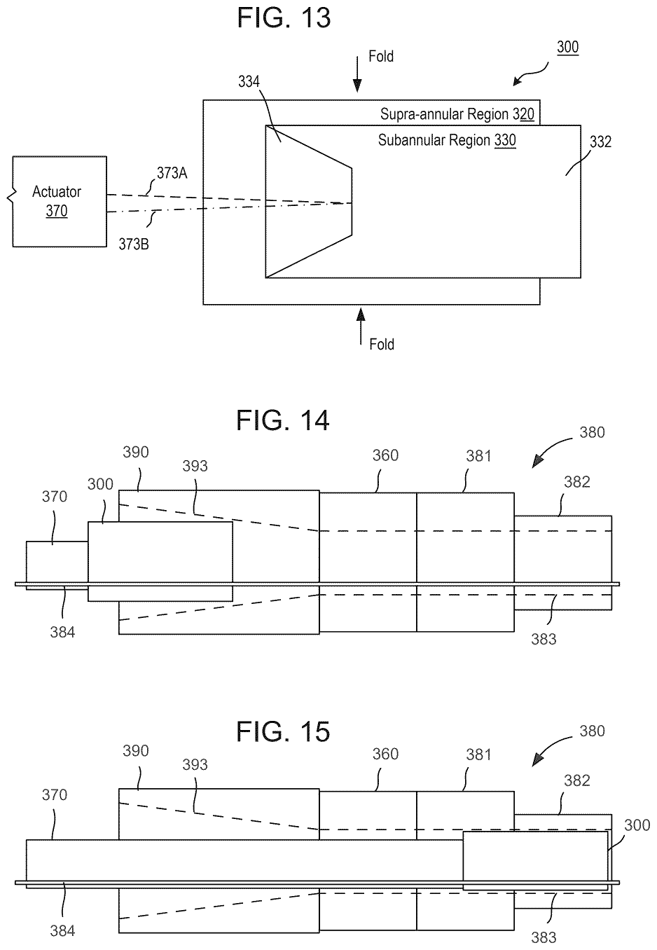

For example, FIGS. 13-15 illustrate a process of delivering the prosthetic valve 300 using a delivery/deployment system 380. As shown, the actuator/control device 370 can be removably coupled to the prosthetic valve 300 prior to loading the prosthetic valve 300 into the delivery/deployment system 380. The delivery/deployment system 380 and/or at least a portion or function thereof can be substantially similar to the delivery system described in the '032 PCT.

With the actuator 370 coupled to the prosthetic valve 300, a user can actuate the actuator 370 by pulling the first tether 373A in a proximal direction, thereby increasing a tension along the first tether 373A. Accordingly, the first tether 373A can pull the proximal anchoring element 334 to the first configuration, as described in detail above. As shown in FIG. 13, with the proximal anchoring element 334 in the first or cinched configuration, a force can be exerted on the prosthetic valve 300 to fold the valve in a direction along the lateral direction. Once cinched and folded, the prosthetic valve 300 can be inserted into a compression device 390 that includes a set of inner walls defining a tapered lumen, as shown in FIG. 14. As such, the inner walls can be configured to compress the prosthetic valve 300 in at least a direction along the central axis as the prosthetic valve 300 is advanced through the compression device 390, as described in detail in the '032 PCT. In some implementations, the folded and/or flipped arrangement of the proximal anchoring element 334 in the first or cinched configuration can be such that resistance and/or hinderances associated with advancing the prosthetic valve 300 in a distal direction through the compression device 390 are limited.

The compression device 390 can be configured to compress the prosthetic valve 300 to the compressed and/or delivery configuration, allowing the prosthetic valve 300 to be advanced into a loading device 360. The loading device 360, in turn, can be coupled to a portion of the delivery/deployment system 380 (e.g., a delivery handle 381 from which a delivery catheter 382 extends). The loading device 360 can be configured to establish hemostasis with a lumen 383 extending through a portion of the delivery/deployment system 380, as described in detail in the '032 PCT. After establishing hemostasis, the loading device 360 can be manipulated to allow the prosthetic valve 300 to be advanced into the lumen 383 of the delivery catheter 382, as shown in FIG. 15. Although not shown in FIGS. 13-15, a distal end of the delivery catheter 382 can be disposed, for example, in a chamber (atrium) of the heart. In this manner, the actuator/control device 370 can advance the prosthetic valve 300 through the delivery catheter 382 and can release the prosthetic valve 300 from the distal end of the delivery catheter 382 into the chamber (atrium) of the heart while the proximal anchoring element 334 is in the first or cinched configuration.

Provided below is a discussion of certain aspects or embodiments of side deliverable transcatheter prosthetic valves (e.g., prosthetic valves) and/or delivery systems and methods for delivering such prosthetic valves. The prosthetic valves (or aspects or portions thereof) described below with respect to specific embodiments can be substantially similar in at least form and/or function to the valves 100, 200, and/or 300 (or corresponding aspects or portions thereof) schematically represented above. Likewise, the delivery systems and/or methods (or aspects or portions thereof) described below with respect to specific embodiments can be substantially similar in at least form, function, and/or process as the delivery/deployment system 180 and/or 380 or process of using such systems (or aspects, portions, and/or processes thereof) schematically represented above. Thus, certain aspects and/or portions of the specific embodiments may not be described in further detail herein.

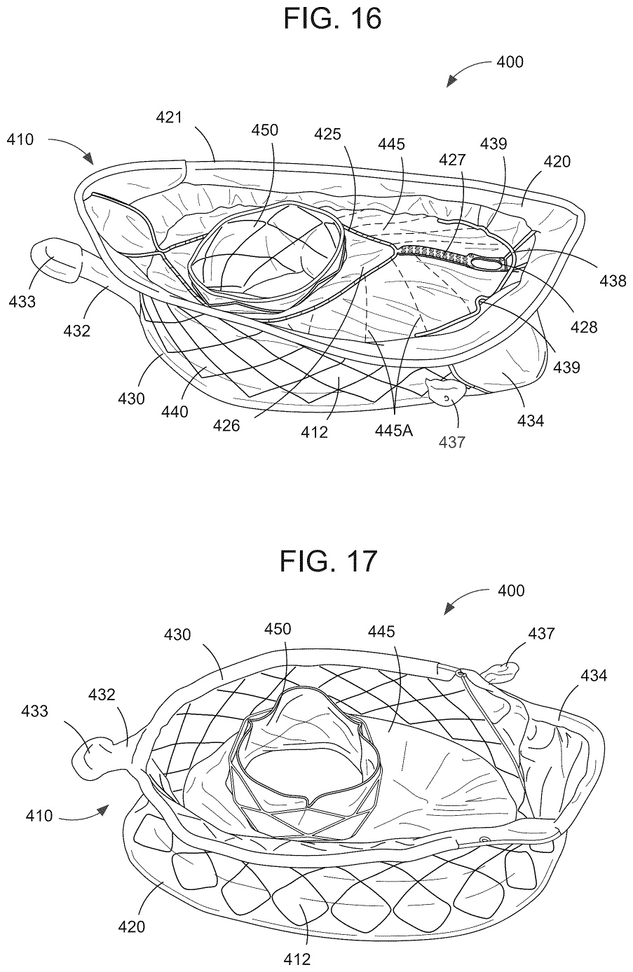

FIGS. 16-25 illustrate a side-deliverable (orthogonally deliverable) transcatheter prosthetic heart valve 400 (also referred to herein as “prosthetic valve” or “valve”), according to an embodiment. FIGS. 16 and 17 are an illustration of a top perspective view and a bottom perspective view, respectively, of the valve 400. In some implementations, the valve 400 can be deployed in, for example, an annulus of a native tricuspid and/or mitral valve. The valve 400 is configured to permit blood flow in a first direction through an inflow end of the valve 400 and to block blood flow in a second direction, opposite the first direction, through an outflow end of the valve 400. For example, the prosthetic valve 400 can be a side deliverable transcatheter prosthetic heart valve configured to be deployed within the annulus of a native tricuspid valve or native mitral valve of a human heart to supplement and/or replace the functioning of the native valve.

The valve 400 is compressible and expandable in at least one direction relative to an x-axis of the valve 400 (also referred to herein as “horizontal axis,” “longitudinal axis,” “long axis,” and/or “lengthwise axis”). The valve 400 is compressible and expandable between an expanded configuration for implanting at a desired location in a body (e.g., a human heart) and a compressed configuration for introduction into the body using a delivery catheter (not shown in FIGS. 16-25). In some embodiments, the horizontal x-axis of the valve 400 is orthogonal to (90 degrees), or substantially orthogonal to (75-105 degrees), or substantially oblique to (45-135 degrees) to a central (vertical) y-axis when in the expanded and/or compressed configuration. Moreover, the horizontal x-axis of the valve 400 in the compressed configuration is substantially parallel to a lengthwise cylindrical axis of the delivery catheter in which the valve 400 is disposed.

In some embodiments, the valve 400 has an expanded or deployed height of about 5-60 mm, about 5-30 mm, about 5-20 mm, about 8-12 mm, or about 8-10 mm, and an expanded or deployed diameter (e.g., length and/or width) of about 25-80 mm, or about 40-80 mm. In some embodiments, the valve 400 has a compressed height (y-axis) and width (z-axis) of about 5-15 mm, about 8-12 mm, or about 9-10 mm. It some implementations, a length of the valve 400 (e.g., along the x-axis) is not compressed or otherwise reduced since it can extend along the length of the central cylindrical axis of the delivery catheter (e.g., the longitudinal or lengthwise axis).

In certain embodiments, the valve 400 can be centric or eccentric (e.g., radially symmetric or radially asymmetric, respectively, along or relative to the y-axis). In some eccentric embodiments, the frame 410 may have a D-shape in cross-section, with a flat portion or surface configured to substantially match an annulus of a native mitral valve at or near the anterior leaflet. In the example shown in FIGS. 16-25, the valve 400 is eccentric with one or more components being offset or asymmetrical region to the y-axis.

FIGS. 16 and 17 show the valve 400 including an annular outer support frame 410 and a collapsible flow control component 450 mounted within the annular outer support frame 410. The annular outer support frame 410 (also referred to herein as “outer frame”) is made from a shape-memory material such as Nickel-Titanium alloy (Nitinol) and is therefore a self-expanding structure from a compressed configuration to an expanded configuration. The outer frame 410 has a transannular member 412 and/or body that circumscribes, forms, and/or defines a central (interior) channel about and/or along the vertical or central axis (y-axis). The outer frame 410 has a supra-annular member 420 attached circumferentially at a top edge of the transannular member 412 and a subannular member 430 attached circumferentially at a bottom edge of the transannular member 412. At least the outer support frame 410 of the valve 400 is covered, wrapped, and/or surrounded by a biocompatible cover 440. The biocompatible cover 440 can be a mesh material, a pericardial tissue, a woven synthetic polyester material, and/or any other suitable biocompatible material such as those described above.

The biocompatible cover 440 disposed on or along the supra-annular member 420 can form a drum 445 that extends between and/or is coupled to an outer loop and an inner loop of the supra-annular member 420. As such, the drum 445 can cover a space not otherwise occupied by the flow control component 450. The drum 445 can have and/or can form a set of spokes 445A that can be used to increase a stiffness of the drum 445. The drum 445 is further shown having an attachment member 438 that can extend along or across a portion of the drum 445 (or supra-annular member 420). As described in further detail here, the attachment member 438 can facilitate a temporary and/or removable attachment to a portion of a delivery/deployment system such as, for example, a control device, actuator, etc.

The supra-annular member 420 is shaped to conform to the native deployment location. In a tricuspid replacement, for example, the supra-annular member 420 or atrial collar can have a tall back wall portion to conform to the septal area of the native valve and can have a distal and proximal portion. The distal portion can be larger than the proximal portion to account for the larger flat space above (atrial) the ventricular outflow tract (VOT) subannular area. In a mitral replacement, for example, the supra-annular member 420 of the outer frame 410 may be D-shaped or shaped like a hyperbolic paraboloid to mimic the native structure. In some embodiments, the supra-annular member 420 of the outer frame 410 can be substantially similar in at least form and/or function to any of the supra-annular members described above. Thus, portions and/or aspects of the supra-annular member 420 may not be described in further detail herein.

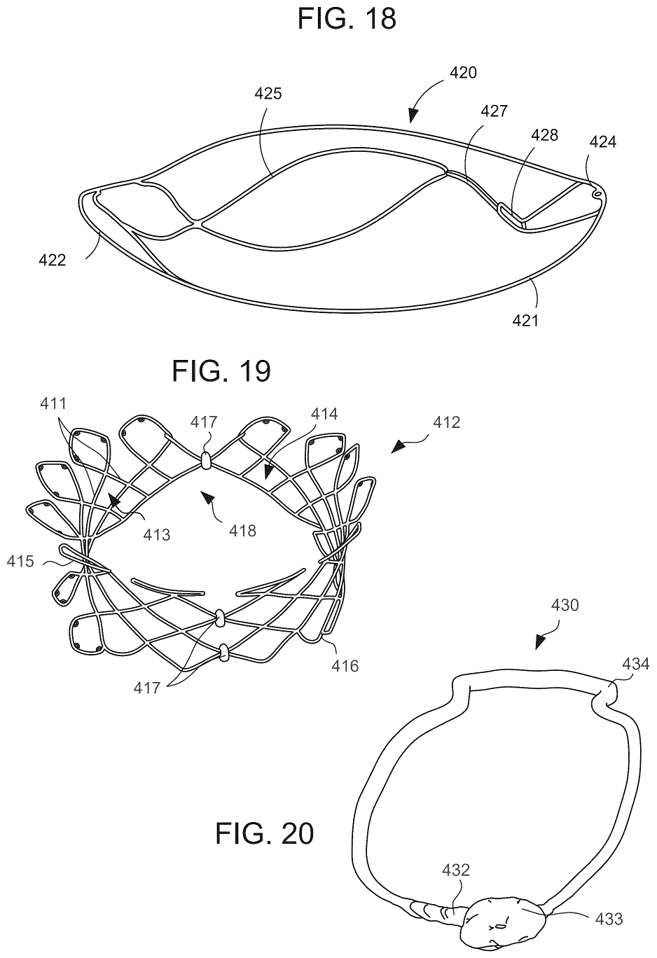

FIG. 18 shows a laser-cut wire frame portion of the supra-annular member 420 (uncovered). As shown, the supra-annular member 420 includes a distal portion 422, a proximal portion 424, an outer loop 421, an inner loop 425, and at least one spline 427. In some embodiments, the outer loop 421 can be shaped and/or sized to engage native tissue. For example, the distal portion 422 of the supra-annular member 420 (formed at least in part by the outer loop 421) is configured to engage distal supra-annular tissue and the proximal portion 424 (formed at least in part by the outer loop 421) is configured to engage proximal supra-annular tissue. The distal and proximal portions 422 and 424 can have a rounded and/or curved shape, wherein a radius of curvature of the proximal portion 424 is larger than a radius of curvature of the distal portion 422. The distal portion 422 can form, for example, a distal anchoring loop 423 that can engage distal supra-annular tissue to at least partially stabilize and/or secure the frame 410 in the native annulus. The proximal portion 424 similarly can form a proximal upper anchoring element that can engage proximal supra-annular tissue to at least partially stabilize and/or secure the frame 410 in the native annulus.

The inner loop 425 of the supra-annular member 420 can be substantially circular, oblong, teardrop-shaped, and/or any other suitable shape. The inner loop 425 can be coupled to and/or suspended from the outer loop by the one or more splines 427. As shown in FIGS. 16 and 17, the inner loop 425 can be coupled to biocompatible material 426, which can be used to couple the inner frame 451 of the flow control component 450 to the inner loop 425 of the support frame 410. In some implementations, suspending the inner loop 425 from the outer loop 421 can, for example, at least partially isolate the inner loop 425 (and the flow control component 450 coupled to the inner loop 425) from at least a portion of the force associated with transitioning the frame 410 between the expanded configuration and the compressed configuration, as described above with reference to the frame 410.

The one or more splines 427 of the supra-annular member 420 can be any suitable shape, size, and/or configuration. For example, in some embodiments, the supra-annular member 420 can include a proximal spline 427 and one or more distal splines 427. The distal splines 427 can couple a distal portion of the inner loop 425 to a distal portion of the outer loop 421. Similarly, the proximal spline 427 can couple a proximal portion of the inner loop 425 to a proximal portion of the outer loop 421. In some embodiments, the proximal spline 427 can be configured to receive, couple to, and/or otherwise engage an actuator, a control device, and/or a portion of a delivery system. For example, the proximal spline 427 includes, forms, and/or can be coupled to a waypoint 428 that can be used to couple and/or to receive one or more portions of the control device and/or delivery system, as described above with reference to the frames 110, 210, and/or 310.

As shown in FIGS. 16-18, in this embodiment, the supra-annular member 420 has a bowed configuration in which the spline 427 protrudes away from other portions of the supra-annular member 420. For example, the laser cut frame of the supra-annular member 420 can be formed with the spline 427 having the bowed configuration. In some implementations, bowed spline 427 can exert a force on the drum 445 that bows the drum 445 and increases a tension across the area of the drum 445. The increase in tension, alone or in conjunction with the spokes 445A, increases a relative stiffness of the drum 445, which can reduce and/or limit an amount of drum deformation during, for example, diastole or systole, thereby enhancing performance of the valve 400 and/or reduce fatigue in or along the drum 445. Said another way, the pressure produced on the atrial side of the drum 445 during contraction of the atrium (diastole) is not sufficient to invert the bowed configuration of the drum 445 (e.g., will not produce an oil-can like deflection) due to the bowed spline 427. The bowed configuration of the drum 445 can also withstand the greater pressure produced on the ventricle side of the drum 445 during contraction of the ventricle (systole) without substantial deflection. Moreover, the bow in the spline 427 can be such that the waypoint 428 is positioned at a desired angle and/or orientation to facilitate the insertion or retrieval of one or more portions of the delivery system through the waypoint 428.

FIG. 19 is a distal perspective view illustrating the transannular member 412 of the outer frame 410 of the valve 400. In some embodiments, the transannular member 412 of the outer frame 410 can be substantially similar in at least form and/or function to any of the transannular regions/members described above. Thus, portions and/or aspects of the transannular member 412 may not be described in further detail herein.

The transannular member 412 can be shaped and/or formed into a ring, a cylindrical tube, a conical tube, and/or any other suitable annular shape. In some embodiments, the transannular member 412 may have a side profile of a concave cylinder (walls bent in), an angular hourglass, a curved, graduated hourglass, a ring or cylinder having a flared top, flared bottom, or both. Moreover, the transannular member 412 can form and/or define an aperture or central channel 414 that extends along the central axis 404 (e.g., the y-axis). The central channel 414 (e.g., a central axial lumen or channel) can be sized and configured to receive the flow control component 450 across a portion of a diameter of the central channel 414. In some embodiments, the transannular member 412 can have a shape and/or size that is at least partially based on a size, shape, and/or configuration of the supra-annular member 420 and/or subannular member 430 of the support frame 410, and/or the native annulus in which it is configured to be deployed, as described above.

The transannular member 412 can be and/or can include a wire frame that is laser cut out of Nitinol or the like and, for example, heat-set into a desired shape and/or configuration. The transannular member 412 or wire frame thereof can include a set struts 411 that form and/or define a set of compressible wire cells 413 having an orientation and/or cell geometry substantially orthogonal to the central axis extending through the central channel 414 to minimize strain along the struts 411 and/or any other portion of the wire frame when the transannular member 412 is in a vertical compressed configuration, a rolled and compressed configuration, or a folded and compressed configuration. As shown in FIG. 19, the transannular member 412 includes a first laser-cut half 415 (e.g., an anterior side) and a second laser-cut half 416 (e.g., a posterior side) that can be formed into a desired shape and coupled together to form the transannular member 412. The first laser-cut half 415 (anterior side) and the second laser-cut half 416 (posterior side) can be coupled at one or more hinge points 417 along a distal portion and a proximal portion of the transannular member 412. More specifically, the first laser-cut half 415 (anterior side) and the second laser-cut half 416 (posterior side) can be coupled along the distal side of the transannular member 412 via two sutures forming two hinge or coupling points 417 and can be coupled along the proximal side of the transannular member 412 via one suture forming a single hinge or coupling point 417.

In some embodiments, forming the transannular member 412 in such a manner can allow the transannular member 412 to bend, flex, fold, deform, and/or otherwise reconfigure (substantially without plastic deformation and/or undue fatigue) in response to lateral folding along or in a direction of a lateral or z-axis and/or vertical compression along or in a direction of the central or y-axis. Moreover, coupling at the hinge points 417 using sutures can allow for a desired amount of slippage between the sutures and the anterior/posterior sides 415/316, which in turn, can limit and/or substantially prevent binding, sticking, and/or failure in response to folding along the lateral or z-axis. In addition, including the single hinge or coupling point 417 at the proximal portion of the transannular member 412 can define a gap or space 418 below the proximal hinge or coupling point 417 that can provide space to allow a proximal anchoring element of the subannular member 430 to transition between a first configuration and a second configuration, as described in further detail herein.

FIG. 20 is a distal perspective view illustrating the subannular member 430 of the outer frame 410 of the valve 400. In some embodiments, the subannular member 430 of the frame 410 can be similar in at least form and/or function to any of the subannular regions and/or members described above. Thus, portions and/or aspects of the subannular member 430 may not be described in further detail herein.

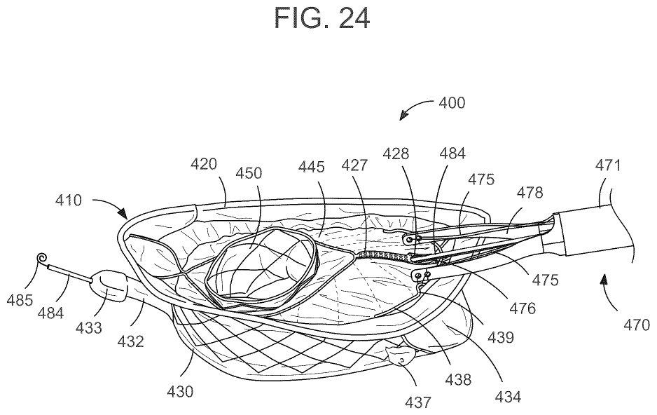

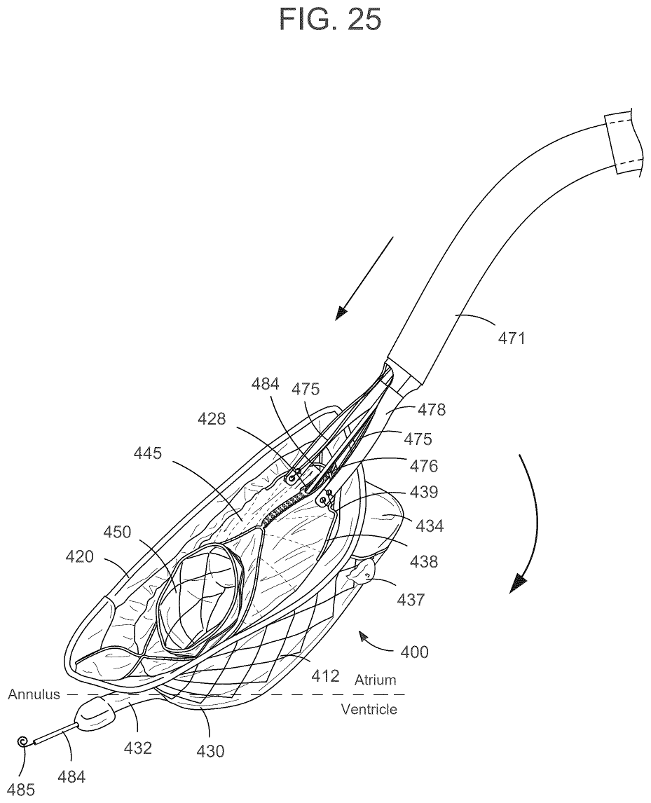

As shown, the subannular member 430 of the frame 410 includes and/or forms a distal portion having a distal anchoring element 432 and a proximal portion having a proximal anchoring element 434. The anchoring elements 432 and 434 are integrally and/or monolithically formed with the subannular member 430. The distal anchoring element 432 and the proximal anchoring element 434 of the subannular member 430 can be any suitable shape, size, and/or configuration. The distal anchoring element 432 is shown as including an atraumatic end that forms a guidewire coupler 433 configured to selectively engage and/or receive a portion of a guidewire catheter 484 (having a guidewire disposed therein) through an opening, hole, aperture, port, etc., defined by the guidewire coupler 433 (see e.g., FIGS. 24 and 25). With the guidewire catheter 484 extending through the guidewire coupler 433, the valve 400 is allowed to be advanced over or along the placed guidewire disposed in the guidewire catheter 484. In some implementations, the guidewire catheter 484 can extend below the valve 400 and beyond the distal anchoring element 432, and can provide a desired stiffness during delivery and/or deployment.

The anchoring elements 432 and/or 434 are configured to engage a desired portion of the native tissue to mount the frame 410 to the annulus of the native valve in which it is deployed. For example, the distal anchoring element 432 can extend (e.g., about 10-40 mm) from the subannular member 430 and into a RVOT or other ventricular position. The distal anchoring element 432 can be shaped and/or biased such that the distal anchoring element 432 exerts a force on the subannular tissue operable to at least partially secure the distal end portion of the frame 410 in the native annulus.

The proximal anchoring element 434 can be configured to engage subannular tissue on a proximal side of the native annulus to aid in the securement of the frame 410 in the annulus. As described above, the subannular member 430 of the frame 410 can be and/or can include, for example, a laser cut wire frame formed of a shape-memory material such as Nitinol, which is heat-set into a desired shape and wrapped in a biocompatible material (e.g., a fabric and/or the like). The proximal anchoring element 434 is configured to transition, move, and/or otherwise reconfigure between a first configuration in which the proximal anchoring element 434 extends from the subannular member 430 a first amount or distance and a second configuration in which the proximal anchoring element 434 extends from the subannular member 430 a second amount or distance. Said another way, the proximal anchoring element 434 can be, for example, a movable anchoring element configured to be moved and/or otherwise transitioned (e.g., by an actuator) between a first configuration and a second configuration to reduce a perimeter of the subannular member 430 during delivery and/or deployment.

As described above, the proximal anchoring element 434 can be in a compressed, contracted, retracted, undeployed, folded, and/or restrained state (e.g., a position that is near, adjacent to, and/or in contact with the transannular member 412 and/or the supra-annular member 420 of the support frame 410) when in the first configuration, and can be in an expanded, extended, deployed, unfolded, and/or unrestrained state (e.g., extending away from the transannular member 412) when in the second state. In some embodiments, the proximal anchoring element 434 can be biased and/or heat-set in the second configuration. Moreover, in some implementations, the space 418 defined by the transannular member 412 of the outer frame 410 is configured to provide sufficient room to allow the proximal anchoring element 434 to transition between the first and second configurations.

The proximal anchoring element 434 can be configured to move in any suitable direction from the first, extended configuration to the second, compressed configuration based at least in part on how the proximal anchoring element 434 is coupled to an actuator and/or the like. For example, the proximal anchoring element 434 can be moved inward toward the inner flow control component 450, moved upward toward the supra-annular member 420 and/or portion thereof, and/or moved toward an anterior side or a posterior side of the valve 400. Moreover, with the transannular member 412 of the frame 410 coupled to the subannular member 430, actuation of an actuator, control device, etc., can, in some instances, move one or more portions of the transannular member 412, as described in further detail herein.

The collapsible (inner) flow control component 450 is mounted within the outer frame 410. The flow control component 450 has a foldable and compressible inner wire frame 451 (also referred to as “inner leaflet frame” or “inner frame”) with two (or more) fold areas, hinge areas, coupling areas, elastically deformable regions, etc. A set of 2-4 flexible leaflets 456 are mounted in or on the inner frame 451. In some embodiments, the flow control component 450 has three leaflets 456 (e.g., components, cusps, pockets, etc.) mounted within the inner frame 451, as described in further detail herein.

The flow control component 450, like the outer frame 410, is foldable and compressible. For example, the inner frame 451 is foldable along or in the direction of a z-axis (e.g., foldable at the fold areas or the like) from a cylindrical configuration to a flattened cylinder configuration (or a two-layer band), where the fold areas are located on a distal side and on a proximal side of the inner frame 451. The flow control component 450, like the outer frame 410, is also vertically (y-axis) compressible to a shortened or compressed configuration. By folding (compressing) in the direction of the z-axis and vertically compressing in the y-axis, the valve 400 is permitted to maintain a relatively large dimension along the horizontal (x-axis). In some implementations, the outer frame 410 and the flow control component 450 are reduced along z-axis until the side walls are in contact or nearly so. This also allows the outer frame 410 and the flow control component 450 to maintain the radius along the horizontal axis (x-axis), to minimize the number of wire cells that can be damaged by forces applied during folding and/or compression when loading the valve 400 into the delivery catheter.

The flow control component 450 has a diameter and/or perimeter that is smaller than a diameter and/or perimeter of the central channel of the outer frame 410. The flow control component 450 is mounted to or within the outer frame 410 such that a central or vertical axis (y-axis) of the inner frame 451 is parallel to the central or vertical axis (y-axis) of the outer frame 410. In some embodiments, the y-axis defined by the inner frame 451 is parallel to but offset from the y-axis defined by the outer frame 410 (see e.g., FIGS. 16 and 17). In some implementations, the drum 445 (or a spacer element) is disposed within and/or across the central channel and can facilitate the mounting of a portion of the flow control component 450 (e.g., an otherwise unsupported portion) to the outer support frame 410 and/or an ingrowth of native tissue over at least a portion of the supra-annular member 420 of the valve 400.

In certain embodiments, the inner frame 451 can have a diameter of about 25-30 mm, the outer frame 410 (or the transannular member 412 thereof) can have a diameter of about 50-80 mm, and the supra-annular member 420 (or atrial collar) extend beyond the top edge of the transannular member 412 by about 20-30 mm to provide a seal on the atrial floor against perivalvular leaks (PVLs). The flow control component 450 and the outer frame 410 can be foldable (e.g., in the direction of the z-axis) and/or compressible (e.g., in the direction of the y-axis) to reduce a size of the entire valve 400 to fit within the inner diameter of a 24-36 Fr (8-12 mm inner diameter) delivery catheter.

FIGS. 21-23 illustrate at least a portion of the flow control component 450 included in the valve 400. For example, FIG. 21 is an illustration of a top perspective view of the inner leaflet frame 451. In some embodiments, the inner leaflet frame 451 is formed of two separate wireframe sheets or members that are coupled at lateral connection points 452 and 453 (e.g., fold areas, elastically deformable regions, coupled edged portions, etc.). The inner leaflet frame 451 is shown in an expanded or cylindrical configuration (e.g., prior to being folded and/or compressed).

Although not shown, the inner leaflet frame 451 can be transitioned from the expanded or cylindrical configuration to an at least partially folded configuration. The inner leaflet frame 451 can have wireframe sidewalls that allow for rotating or hinging at least at the lateral connection points 452 and 453. The inner leaflet frame 451 can be configured to fold in response to the valve 400 being folded and/or compressed for delivery. When transitioned, for example, to a completely folded configuration, the wireframe sidewalls can be rotated, hinged, and/or folded at their lateral connection points 452 and 453. In addition, the inner leaflet frame 451 can be vertically compressed into a compressed configuration. The wireframe sidewalls can form cells (e.g., diamond-shaped cells or the like) that can be oriented in a direction of compression to allow for elastic compression of the inner frame 451. In some embodiments, the inner frame 451 can be vertically compressed into a pleated or accordion (compressed) configuration.

In some embodiments, the inner leaflet frame 451 of the flow control component 450 can be formed from a linear wireframe or laser cut sheet prior to being further assembled into a cylinder structure (e.g., as shown in FIG. 21). The inner leaflet frame 451 can be formed into the cylinder structure or configuration (or a conical structure or configuration) with edge portions of the linear wireframe sheet being connected or coupled at the lateral connection points 452 and 453 (e.g., hinge areas, fold areas, etc.). Moreover, the inner leaflet frame 451 can be expanded (e.g., driven, formed, bent, etc.) from the linear sheet configuration into the cylinder structure or configuration. While the inner leaflet frame 451 is shown as including two wireframe sheets, members, and/or halves that are coupled at and/or coupled to form two hinge points, in some embodiments, an inner leaflet frame can be formed from a single component or more than two components that are heat-set, worked, and/or otherwise coupled to form and/or define one or more hinge points. For example, an inner leaflet frame can be formed from a single Nitinol tube and can have hinge points that are formed by heat-setting the material in a desired manner. As another example, an inner leaflet frame can be made from a sheet of material (e.g., Nitinol) and formed into a substantially cylindrical shape with free ends of the material being coupled (e.g., via sutures) to form a single hinge point. In such examples, a second hinge point can be formed opposite the sutured hinge point by heat-setting or working the material in a desired manner. As still another example, an inner leaflet frame can be made from more than two sheets or members, which are coupled together (e.g., via sutures) to form a corresponding number of hinge points.

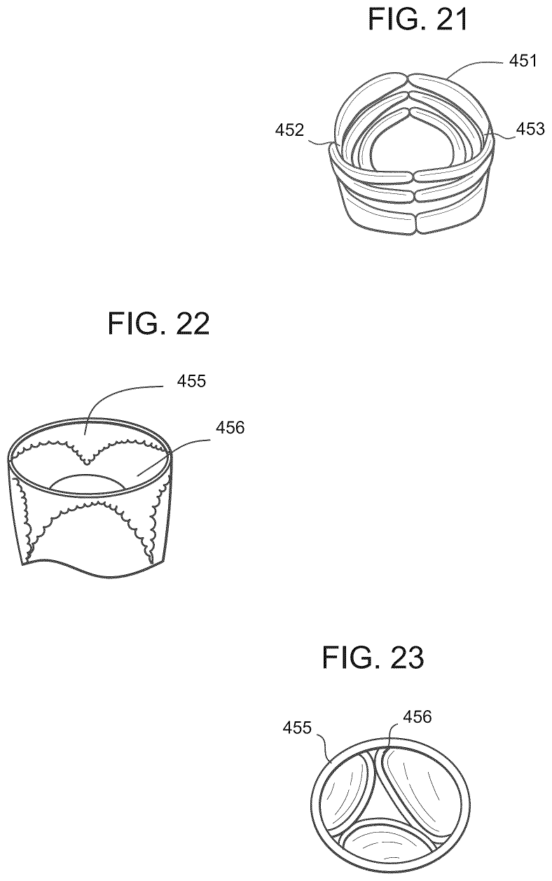

FIGS. 22 and 23 are a side perspective view and a bottom view, respectively, that illustrate a structural band 455 of pericardial tissue with leaflet 456 (e.g., components, cusps, pockets, etc.) sewn into the structural band 455. The structural band 455 and leaflets 456 are shown before assembly and/or mounting on and/or into the inner frame 451 to form the collapsible (foldable, compressible) flow control component 450. The structural band 455 formed of pericardial tissue with the leaflets 456 sewn into the structural band 455, after assembly into the cylindrical leaflet configuration, the leaflets 456 being disposed on an inner surface of the structural band 455. The leaflets 456 can be sewn into the structural band 455 such that an open edge extends outward, and a sewn edge forms a closed top parabolic edge providing attachment. The cylindrical structural band 455 and leaflets 456 are shown in FIG. 23 with partial coaptation towards forming a closed fluid-seal. Although not show, the cylindrical structural band 455 can be mounted to or in the inner leaflet frame 451 to collectively form the flow control component 450, which in turn, is mounted to the inner loop 425 of the supra-annular member 420 of the outer support frame 410, as described in detail above with reference to FIGS. 16 and 17.

FIGS. 24 and 25 are elevated perspective side views showing the prosthetic valve 400 removably coupled to an actuator or control device 470 (referred to as “actuator” or “control device” interchangeably) used to advance, control, and/or retract the valve through a delivery catheter and/or to actuate one or more portions of the valve 400 such as at least the subannular member 430 of the valve frame 410, as described herein. The actuator or control device 470 and/or at least a portion thereof includes a control catheter 471 with a connection member 478 coupled to and/or disposed at a distal end. The control catheter 471 can be, for example, a multi-lumen steerable catheter, having one or more components of the control device 470 extending therethrough, as described in detail in the '032 PCT incorporated by reference above. The connection member 478 is removably coupleable to the supra-annular member 420 of the valve frame 410 and thus, connects the valve 400 to the control catheter 471. As described in further detail herein, the control catheter 471 can be manipulated to, for example, advance the prosthetic valve 400 through a delivery catheter (not shown), control or steer the prosthetic valve 400 during deployment, retrieve and/or withdraw the prosthetic valve 400 into the delivery catheter (e.g., after at least partial deployment), and/or the like.

FIG. 24 shows the connection member 478 having a wishbone or yoke configuration, though other configurations are possible. As such, the connection member 478 can have a first portion, side, and/or arm and a second portion, size, and/or arm opposite the first portion, side, and/or arm. The connection member 478 can be configured to transition between an expanded configuration and a compressed configuration to, for example, allow the control catheter 471 (and the connection member 478 disposed at the distal end thereof) to be advanced through a delivery catheter. The connection member 478 can be formed from any suitable material such as a shape-memory allow like nitinol or the like.

In some embodiments, the connection member 478 can be in contact with and/or removably coupled to the drum 445 of the supra-annular member 420 and/or any other suitable portion of the frame 410 or valve 400. The connection member 478 can removably couple to the valve 400 via sutures, tethers, cables, clips, couplers, and/or any other removable coupling. For example, in some embodiments, the control device 470 can include a set of tethers 475 extending from one or more lumen defined by the control catheter 471. The tethers 475 are shown extending from the control catheter 471, looping through a set of openings defined along or by each side or arm of the connection member 478 (yoke), looping around one or more attachment members 438 of the valve 400, and extending back into the corresponding lumen of the control catheter 471. The attachment member(s) 438 can be formed by, coupled to, and/or extend from the supra-annular member 420 (e.g., the drum 445). In some embodiments, the attachment member 438 of the valve 400 can be a tether, suture, cable, frame structure, and/or the like that can be coupled to and/or extend from a wire frame portion of the supra-annular member 420 or, for example, the drum 445 (or other biocompatible covering). Moreover, the attachment member 438 can form a pair of loops 439 or the like around which the tethers 475 of the control device 470 can be routed or looped.

The looped arrangement of the tethers 475 through and/or around the connection member 478 and the attachment member 438 of the valve 400 is such that each of the proximal end and the distal end of the tether 475 extends through and outside of (e.g., proximal to) the patient. As such, a proximally directed force can be exerted on each of the proximal end and the distal end of the tether(s) 475 to increase a tension along the tether 475, which pulls the connection member 478 toward the drum 445, thereby securing the connection member 478 to the valve. Conversely, a proximally directed force exerted on only one of the proximal end or the distal end of the tether(s) 475 can disengage the tether(s) 475 from the connection member 478 and can withdraw the tether(s) 475 from the control device 470, which in turn, can allow the connection member 478 to be decoupled or removed from the valve 400.

FIG. 24 further shows the guidewire catheter 484 of the delivery system extending through, for example, the waypoint 428 or opening in the supra-annular member 420 and/or drum 445 thereof and extending through the guidewire coupler 433 of the distal anchoring element 432. The guidewire catheter 484 can extend below the flow control component 450 of the valve 400. Prior to and/or as a part of delivery, the guidewire catheter 484 can be advanced and/or inserted through the valve 400 and advanced over the guidewire 485 that is already placed in a desired position within the heart. As such, delivering the valve 400 in a compressed configuration through a delivery catheter includes advancing the guidewire catheter 484 along the guidewire 485. The guidewire catheter 484 can extend through and beyond the guidewire coupler 433 of the distal anchoring element 432 (e.g., a distal end of the guidewire catheter 484 can be distal to the guidewire coupler 433 by about 0.1 cm to about 1.0 cm, or more).

The guidewire catheter 484 can be sufficiently stiff to, for example, limit and/or define (at least in part) a range of motion of the valve 400 during delivery. For example, the guidewire catheter 484 can define an axis about which the valve 400 can rotate during delivery but can substantially limit or oppose movement of the valve 400 in other directions. In some implementations, the arrangement of the connection member 478 (e.g., yoke) and the guidewire catheter 484 can allow for greater control of a position of the valve 400 during delivery. The guidewire catheter 484 and/or one or more portions of the valve 400 (e.g., the subannular member 430) can also include radiopaque markers allowing for enhanced visualization during image guided delivery. For example, in some instances, a radiopaque marker or wire can be placed relative to an annular plane of the native valve and can define a landmark during image guided delivery. In such instances, the radiopaque markers on the guidewire catheter 484 and/or other portion(s) of the valve 400 (e.g., the subannular member 430) can be used to align, orient, locate, index, etc. the valve 400 relative to the landmark, which in turn, corresponds to the annular plane of the native valve. Thus, image guided delivery can allow a user to visualize the valve 400 during delivery and/or deployment and can allow the user to visualize when the valve 400 has been seated in the annulus (e.g., the radiopaque marker bands of the valve 400 are below or in a subannular direction relative to the radiopaque landmark).

FIG. 24 further shows at least one tether 476 (e.g., tethers, sutures, cables, tensile members, and/or the like) extending from the control catheter 471 (e.g., through one or more lumen thereof) and through the waypoint 428. The control device 470 can include a single tether or multiple tethers (e.g., one tether, two tethers, three tethers, four tethers, five tethers, six tethers, seven tethers, eight tethers, nine tethers, ten tethers, or more, each of which can be removably coupled to one or more attachment points on the valve 400). The tether(s) 476 can be configured to actuate and/or transition one or more portions of the valve 400 such as, for example, the subannular member 430 and/or at least the proximal anchoring element 434 thereof. In some embodiments, the tether(s) 476 can extend through the waypoint 428, can be looped around and/or through attachment points along the subannular member 430 or at least the proximal anchoring element 434, and then can be routed back through the waypoint 428 and the control catheter 471. As such, both ends of each tether 476 are outside the patient, thereby allowing manipulation of the tether(s) 476 to actuate the valve 400 and/or to transition a shape of the proximal anchoring element 434, the subannular member 430, and/or other portions of the valve 400 to facilitate seating at least a proximal side of the valve 400 into the native annulus. Said another way, increasing an amount of tension along the tether(s) 476 can be operable to transition at least the subannular member 430 (or portion thereof) between a first configuration and a second configuration. As such, the tether(s) 476 can be actuated (or placed in tension) and/or released in a manner similar to that described above with reference to the tether(s) 475. In other embodiments, a distal end portion of the tether(s) can form a loop or the like that can be disposed over or about a portion of the guidewire catheter 484 (e.g., a portion proximal to the guidewire coupler 433). In such embodiments, this arrangement can, for example, releasably secure or anchor the distal end portion of the tether(s) 476, as described above with reference to the valves 100, 200, and/or 300.

FIG. 25 shows the valve 400 and the control device 470 during deployment into a native annulus of the heart. As described above, the control device 470 can advance the valve 400 through the delivery catheter 482 and into the atrium of the heart. In some implementations, the delivery catheter 482 can remain in a substantially fixed position relative to the atrium, or the IVC through which it extends, while a distal end of the control device 470 and the valve 400 are advanced along the guidewire catheter 484 in a distal direction relative to (e.g., away from) the delivery catheter 482 toward the annulus. As such, a length of a portion of the control catheter 471 that is distal to the delivery catheter 482 increases. Because the valve 400 is no longer constrained by the delivery catheter 482, releasing the valve 400 into the atrium allows the valve 400 to transition from the compressed configuration to the expanded configuration.

The control device 470 can be manipulated or steered to place the valve 400 in the expanded configuration at a desired deployment angle in which the distal anchoring element 432 is positioned below the annulus and near, adjacent, and/or at least partially in, for example, a ventricular outflow tract (e.g., the RVOT). At the deployment angle, the supra-annular member 420 (or region) of the valve frame 410 and a least a proximal portion of the subannular member 430 (or region) of the valve frame 410 remain in the atrium. In some implementations, a distal surface of the transannular member 412 of the valve frame 410 can be placed in contact with native tissue forming a distal surface or wall of the annulus. In some instances, the valve 400 can be temporarily maintained in this partially deployed position (e.g., at the deployment angle) allowing a user to verify the positioning of the valve 400 relative to the angle (e.g., by visualizing radiopaque markers under fluoroscopy) and/or allowing blood flow through the annulus to start to transition from flowing entirely through the native valve to flowing through the flow control component 450. In some instances, this can also allow a user to verify that the flow control component 450 is functioning in a desired manner prior to completely seating the valve 400 in the annulus.

Once the position and/or function of the valve 400 is verified, the control device 470 can be manipulated and/or steered to pivot the valve 400 relative to the annulus such that the proximal portion of the valve 400 is inserted and/or dropped into the annulus. Although not shown in FIG. 25, the proximal anchoring element 434 can be in and/or can be transitioned to a compressed, actuated, and/or cinched configuration (e.g., via the tether(s) 476 or any other suitable actuator) such that a perimeter and/or extent of the subannular member 430 of the valve frame 410 is less than a perimeter or extent of the annulus. In some implementations, the proximal anchoring element 434 can be transitioned to the cinched configuration after the valve is released from the distal end of the delivery catheter and allowed to expand to the expanded configuration. For example, the proximal anchoring element 434 can be actuated to the cinched configuration after verifying the function of the flow control component 450.

In other implementations, the proximal anchoring element 434 can be transitioned to the cinched configuration prior to compressing the valve 400 to the compressed configuration. For example, after coupling the control device 470 to the valve 400, the tether(s) 476 can be actuated to increase a tension along the tether(s) 476, which in turn, can pull, flip, fold, and/or otherwise transition the proximal anchoring element 434 from the uncinched configuration to the cinched configuration. In some instances, once the proximal anchoring element 434 is in the cinched configuration, the tether(s) 476 can be at least temporarily locked and/or secured (e.g., via a portion of the delivery system, control device, and/or the like) to at least temporarily maintain the proximal anchoring element 434 in the cinched configuration. The valve 400 can then be folded and/or compressed in the lateral direction and compressed in the axial direction to place the valve 400 in the delivery configuration (e.g., a cinched and compressed configuration). As described in detail above, the valve 400 in the delivery configuration can then be loaded into the delivery system and the control device 470 can be used to advance the valve 400 in the delivery configuration along the guidewire catheter 484 and through the lumen of the delivery catheter. Moreover, the valve 400 can be configured to expand to the expanded or at least partially expanded configuration when released from the distal end of the delivery catheter while the proximal anchoring element 434 remains or substantially remains in the cinched configuration.

With the proximal anchoring element 434 in the cinched configuration, the control device 470 and/or the control catheter 471 can be manipulated and/or steered such that a distally directed force exerted by a user on the control device 470 results in the connection member 478 pushing the proximal portion of the valve 400 in a direction of the annulus. In some implementations, the pivoting the valve 400 can include “steering” the control catheter 471 such that a distal portion of the control catheter 471 bends relative to a distal end of the delivery catheter 482, allowing the connection member 478 to seat the proximal portion of the valve 400 in the annulus. Once seated, the control device 470 can be actuated and/or manipulated to release the tension along one or more of the tethers 476 (e.g., one or more cinch tethers) allowing the proximal anchoring element 434 to transition to or toward the expanded, uncinched configuration. In some embodiments, the control device 470 can include one or more tethers that can be actuated after the valve 400 is seated to pull or otherwise aid the transition of the proximal anchoring element 434 to the expanded, uncinched configuration, as described in detail above with reference to at least the valve 300. The delivery/deployment system including the control device 470 (and tethers 476 thereof) can then be decoupled from the valve 400 and retracted/removed from the patient, leaving the prosthetic valve 400 in the annulus, as described above with reference to the valves 100, 200, and/or 300.

As described above, any of the prosthetic valves described herein can include a subannular region, member, element, etc. that can be actuated, cinched, and/or otherwise transitioned between two or more configurations to facilitate delivery, deployment, and/or seating of the prosthetic valve in a native annulus. In some implementations, the subannular region, member, element, etc. can be similar to and/or can include features similar to any of those described above and/or described in the '996 PCT and/or the '032 PCT incorporated by reference hereinabove.

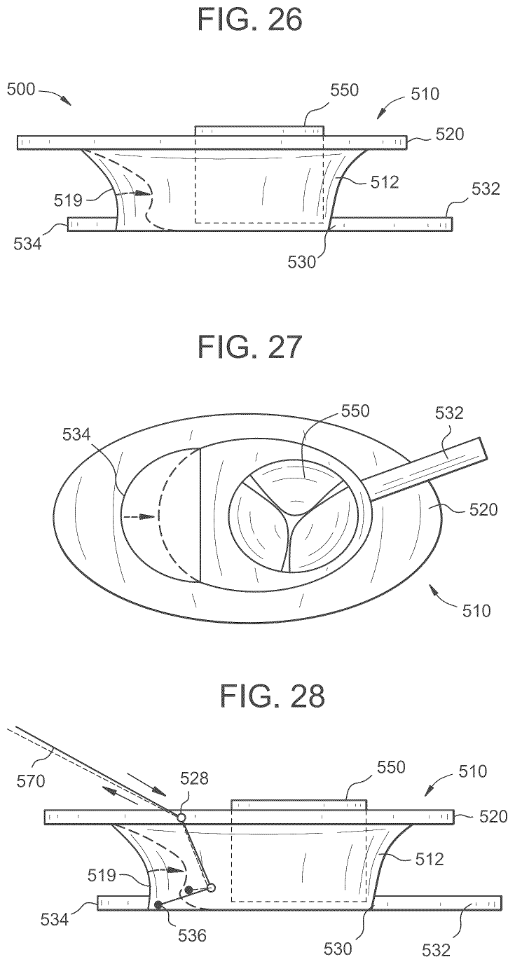

For example, FIGS. 26-28 illustrate a prosthetic valve 500 according to an embodiment. The valve 500 includes an outer support frame 510 and a flow control component 550 mounted therein. The outer support frame includes a supra-annular member 520, a subannular member 530, and a transannular member 512 coupled therebetween. The subannular member 530 includes a distal anchoring element 532 and a proximal anchoring element 534.

FIG. 26 is a schematic side view showing how the perimeter (circumference) of the transannular member 512 of the valve 500 can be cinched inward (as indicated by the arrow and dashed line). FIG. 27 is a schematic bottom view showing how the perimeter (circumference) of the subannular member 530 of the valve 500 can be cinched, flipped, and/or folded inward (as indicated by the arrow and dashed line). The cinching of the transannular member 512 and/or the subannular member 530 allows the valve 500 to be designed with an over-sized transannular and subannular circumference (e.g., 2-20%, often 10-15%) to promote a tight fit of the valve 500 within the native annulus and provide a good seal against perivalvular leakage (PVLs). In some implementations, the cinching process pulls a proximal sidewall 519 inwards and reduces the circumference of the transannular member 512 and/or subannular member 530. This allows the oversized valve to drop into the native annulus during deployment of the valve 500. Then, once the valve 500 is seated as desired, the transannular member 512 and/or subannular member 530 is pushed/pulled back out and/or otherwise allowed to expand to its full or nearly full, circumference, and thereby form a tight, sealed fit of the prosthetic valve 500 in the native annulus. In some implementations, the proximal anchoring element 534 of the subannular member 530 can also be cinched, flipped, and/or folded inward and/or upward with the transannular member 512 and/or independent of the transannular member 512.

FIG. 28 is a schematic side view of the valve 500 at least temporarily coupled to an actuator and/or control device 570, shown as a non-limiting example of a mechanism for performing a cinching process to reduce the perimeter (circumference) of the transannular member 512 and/or the subannular member 530. The actuator/control device 570 (referred to as “actuator 570” or “control device 570” interchangeably) can be and/or can include one or more tethers that travel from a delivery catheter and/or control catheter (not shown), through a way-guide, waypoint, attachment point, through hole, eyelet, and/or any other suitable component (referred to herein as “waypoint 528”) of the valve 500, and couple to one or more attachment points 536 along the subannular member 530 and/or transannular member 512. In this embodiment, the actuator 570 (e.g., cinch tether(s)) travel through the supra-annular member 520 by way of the waypoint 528 and are secured and/or mount to the proximal sidewall 519 of the transannular member 512 and/or the proximal anchoring element 534 of the subannular member 530 at one or more attachment points 536.

In some embodiments, the cinch tether(s) can be secured along a strut of the wireframe of the transannular member 512 (e.g., rather than spanning across the wire cells). In some instances, such a configuration can limit and/or can substantially prevent a cinching of the valve 500 in an axial direction (e.g., cinching in a manner that reduces a height of the valve 500). As such, actuating the actuator 570 (e.g., pulling the cinch tether(s) proximally, towards the operator) pulls the proximal sidewall 519 of the transannular member 512 inwards and reduces the circumference of the transannular member 512 (e.g., without substantially reducing a height of the valve 500), which allows the oversized valve 500 to drop into the native annulus during deployment of the valve 500. In some implementations, the actuator 570 can be actuated to cinch the transannular member 512 and/or the subannular member 530 prior to compressing the valve 500 for delivery through a delivery catheter. In such implementations, after cinching the transannular member 512 and/or the subannular member 530, the valve 500 can be folded and/or compressed in a direction along a lateral axis of the valve 500 and compressed in a direction along a central axis of the valve 500, thereby placing the valve 500 in a delivery configuration (e.g., a cinched and compressed configuration). As described in detail above, the valve 500 in the delivery configuration can be advanced through the delivery catheter (e.g., via a portion of the actuator/control device 570) and released from a distal end of the delivery catheter and into a chamber of the heart (e.g., an atrium). When released, the valve 500 can transition to an expanded configuration while the transannular member 512 and/or the subannular member remain or substantially remain in the cinched configuration, allowing the oversized valve 500 to drop into the native annulus.

Then, once the valve 500 is seated as desired, the actuator 570 can be actuated (e.g., the cinch tether(s) can be advanced or retracted/released) or released to push the proximal sidewall 519 of the transannular member 512 back out and/or to otherwise allow the transannular member 512 to expand to its full or nearly full, circumference, and thereby form a tight, sealed fit of the prosthetic valve 500 in the native annulus. Similarly, the proximal anchoring element 534 can be actuated (e.g., by the actuator 570) and/or released along with the transannular member 512 or independent of the transannular member 512. In some embodiments, first portion of the actuator 570 (e.g., a first tether) can be actuated to cinch the proximal sidewall 519 and/or the proximal anchoring element 534 and a second portion of the actuator 570 (e.g., a second tether) can be actuated to pull, release, and/or return the proximal sidewall 519 and/or the proximal anchoring element 534 to an uncinched or expanded configuration.

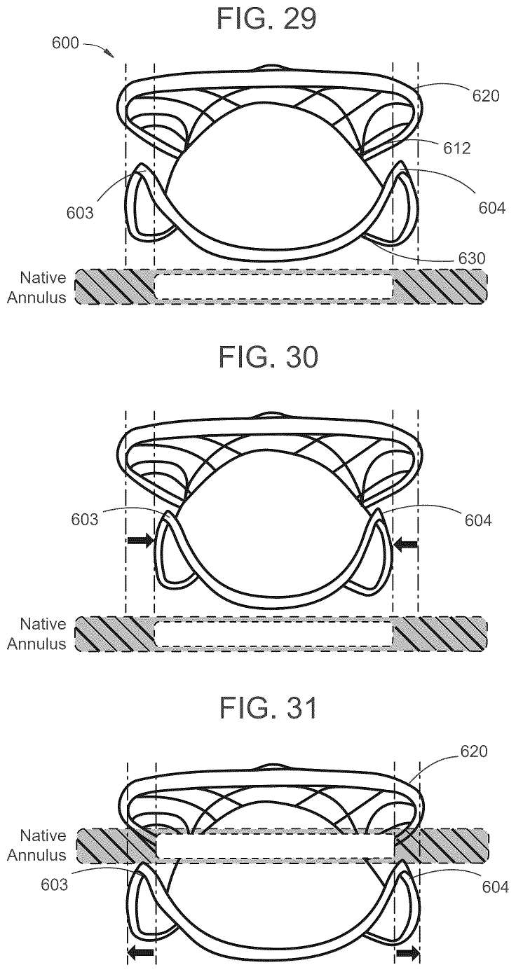

FIGS. 29-31 illustrate a prosthetic valve 600 during at least a portion of a process for deploying the prosthetic valve 600 in a native annulus according to an embodiment. FIGS. 29 and 30 show the prosthetic valve 600 having an outer support frame that includes a supra-annular member 620, a subannular member 630, and a transannular member 612. The subannular member 620 can include side portions or flares 603 and 604 on the freewall (left) and the septal (right) sides, respectively, that can transition between extended (FIG. 29) and retracted or cinched positions (FIG. 30) to allow the valve 600 to slide through the native annulus. FIG. 31 shows the valve 600 deployed, seated, and/or otherwise extending through the native annulus and the side portions and/or flares 603 and 604 on the freewall (left) and the septal (right) sides, respectively, transitioned from the retracted positions (FIG. 30) to or toward the extended positions (FIG. 31). As such, the side portions and/or flares 603 and 604 extend radially to provide an anchoring mechanism (e.g., against native tissue that forms and/or defines the native annulus). The side portions and/or flares 603 and 604 can be actuated using any of the actuation devices and/or methods described herein (e.g., tethers, cables, etc.).

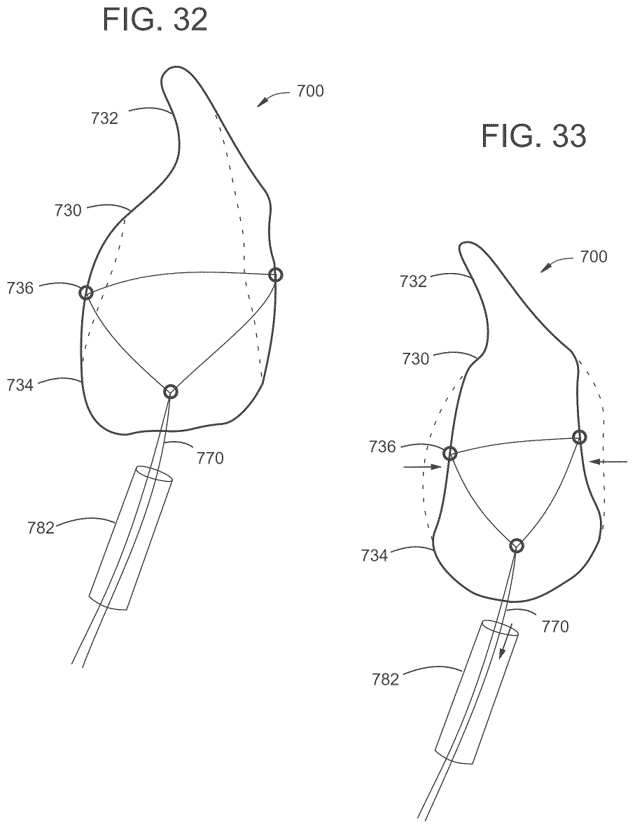

FIGS. 32 and 33 are bottom view illustrations showing a portion of a prosthetic valve 700 coupled to an actuator and/or control device 770 according to an embodiment. The prosthetic valve 700 has a subannular member 730 that can have and/or can form a wire loop (and attached sidewalls), which is/are drawn inward to reduce the perimeter or circumference of the valve body to facilitate deployment of the valve 700 in the native annulus. FIGS. 32 and 33 show that the valve 700 can be removably coupled to the actuator/control device 770, which can be and/or can include one or more tethers, sutures, tensile members, cords, cables, etc. and advanced through a delivery catheter 782 to be positioned in a chamber of the heart. The actuator/control device 770 (referred to as “actuator 770” or “control device 770” interchangeably) extends through the delivery catheter 782 and can be used to actuate the subannular member 730 and/or any other suitable portion of the valve 700 (e.g., by pulling the actuator 770 in a proximal direction). The actuator 770 can couple to the subannular member 730 via attachment points 736 that are positioned at any suitable location(s) along the subannular member 730. In some embodiments, the placement of the attachment points 736 can at least partially control a way in which the subannular member 730 is transitioned and/or cinched, as described herein with reference to specific embodiments. The subannular member 730 is shown with a distal anchoring element 732 and a proximal anchoring element 734. In some implementations, the actuator 770 may also be used to actuate the proximal anchoring element 734 and/or the distal anchoring element 732. In some implementations, once the valve 700 is deployed in an annulus of a native valve, the actuator 770 can be removed or decoupled from the valve 700 and retracted through the delivery catheter 782.

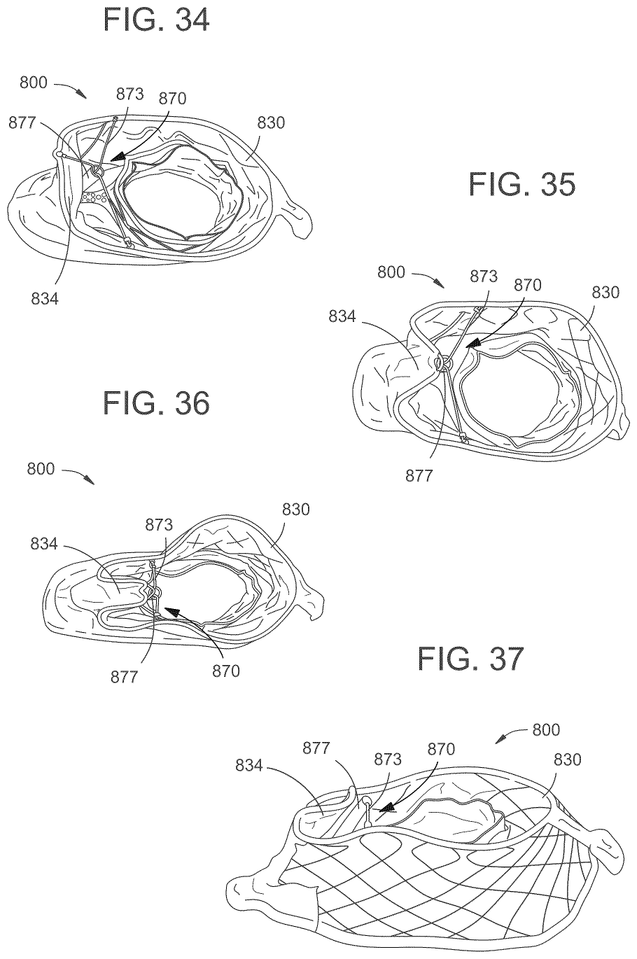

FIGS. 34-37 are bottom perspective views of a prosthetic valve 800 removably coupled to an actuator 870 used to actuate one or more portions of the valve 800 according to an embodiment. The valve 800 has a subannular member 830 that can have and/or can form a wire loop (and attach to sidewalls), which is/are drawn inward to reduce the perimeter or circumference of at least the subannular member 830 to facilitate delivery and/or deployment of the valve 800 in the native annulus. In this embodiment, the actuator 870 can be and/or can include a set of tethers, tensile members, sutures, cables, and/or any other suitable connectors (referred to herein as “tethers 873”) that can be attached to one or more attachment points along the subannular member 830 (e.g., a proximal anchoring element 834 of the subannular member 830 and/or any other suitable portion of the subannular member 830). The actuator 870 can also include and/or can be at least partially disposed in a catheter 877 that can be inserted through a waypoint, opening, attachment point, through hole, etc. formed by a supra-annular member of the valve frame. In some implementations, the actuator 870 can be and/or can include separate tethers 873 used to actuate (e.g., fold) the proximal anchoring element 834, to actuate (e.g., fold) a septal wall sidewall of the valve 800, and/or to actuate (e.g., fold) a freewall sidewall of the valve 800.

FIGS. 34-37 show a set of tethers 873 of the actuator 870 extending from the catheter 877 that extends through and/or is at least partially disposed below a supra-annular member of the valve frame. For example, the catheter 877 can be a relatively small waypoint catheter and/or any other suitable tube, conduit, port, etc. through which the tethers 873 can be run. A proximal end of the tethers 873 (not shown) can be actuated outside of the patient to move a distal end of the tethers 873 relative to a distal end of the catheter 877 (e.g., in a direction toward and/or away from the distal end of the catheter 877), which in turn, can manipulate a shape of the proximal anchoring element 834, the subannular member 830, and/or any suitable portion of the valve 800 to facilitate seating a proximal side of the valve 800 into the native annulus. In some implementations, during delivery, the waypoint catheter 877 can be in a lumen of a delivery catheter and maintained proximal to the compressed valve 800 to avoid having the waypoint catheter 877 stacked on top of the compressed valve 800 within the delivery catheter.

The actuator 870 can include, for example, a single tether or multiple tethers (e.g., one tether, two tethers, three tethers, four tethers, five tethers, six tethers, seven tethers, eight tethers, nine tethers, ten tethers, or more, each of which can be removably coupled to one or more attachment points on the valve 800). The actuator 870 and/or the tethers 873 may be equipped with disconnection elements to allow the actuator 870 and/or tethers 873 to be withdrawn after the valve 800 is deployed and secured in the native annulus. In some embodiments, a distal end portion of the tethers 873 can form a loop or the like allowing the distal end portion of the tethers 873 to be looped around one or more portions of the valve 800, a release pin, a guidewire or guidewire catheter, and/or the like. In such embodiments, actuating one or more portions of the valve 800, releasing the pin, and/or retracting the guidewire/guidewire catheter can be operable to release the distal end portion of the tethers 873, thereby allowing the tethers 873 to be decoupled from the attachment points. The catheter 877 may also be included and/or housed in or extend through a portion of the delivery system such as, for example, a pusher catheter, control catheter (e.g., a multi-lumen control catheter), and/or the like, whereby the catheter 877 can drop through the waypoint, through hole, opening, etc. of the valve 800 to a subannular position, while the pusher catheter, control catheter, and/or other portion of the delivery system is too large to pass through the waypoint. As such, the pusher catheter, control catheter, and/or other portion of the delivery system can be used to control a placement of at least a portion of the valve 800. For example, the pusher catheter, control catheter, and/or other portion of the delivery system can be used to push down onto a surface of the supra-annular member to seat the proximal side of the valve 800 in the native annulus while the subannular member 830 is in an actuated configuration.

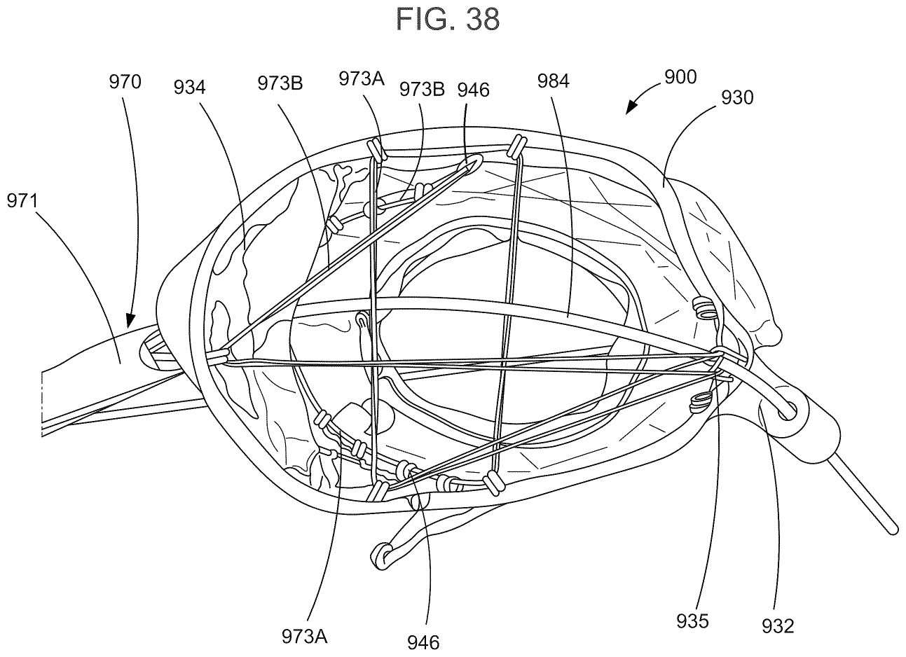

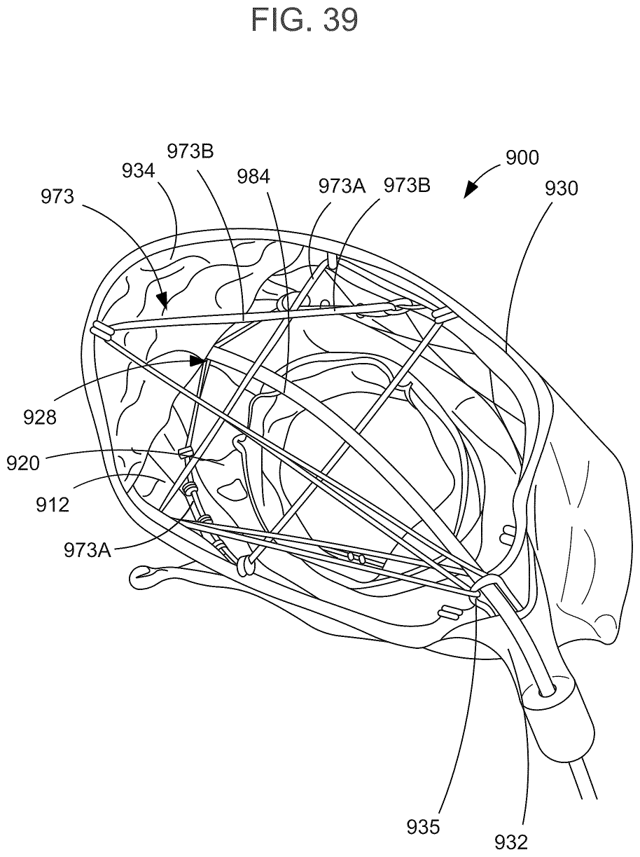

FIG. 34 is a bottom perspective view of the valve 800 and the actuator 870 and shows the subannular member 830 in an at least partially extended or unactuated configuration. FIG. 35 is a bottom perspective view of the valve 800 and the actuator 870 and shows the subannular member 830 partially actuated such that, for example, the tethers 873 pull the proximal anchoring element 834 of the subannular member 830 toward the catheter 877 and/or the flow control component of the valve 800. FIG. 36 is a bottom perspective view of the valve 800 and the actuator 870 and shows the tethers 873 pulled into the catheter 877 (or substantially into the catheter 877) to place the subannular member 830 in a compressed, folded, cinched, and/or actuated configuration such that the proximal anchoring element 834 and, for example, a proximal portion of a septal sidewall and a free sidewall of the valve 800 are drawn toward the catheter 877 and/or the flow control component of the valve 800. FIG. 37 is a bottom-side perspective view of the valve 800 and the actuator 870 and shows the subannular member 830 in the actuated configuration, the catheter 877 extending below the subannular member 830 of the valve frame, and the tethers 873 of the actuator 870 retracted or pulled toward and/or into the catheter 877.

FIG. 37 shows that the catheter 877 can also be used to pull the valve 800 down into the ventricle (e.g., via the retracted tethers 873), avoiding the need to push a compressible valve into the native annulus. More specifically, extending the waypoint catheter 877 through the waypoint such that the end portion is below the supra-annular member of the valve 800 (and, in some instances, below the subannular member 830 of the valve 800) can reduce and/or limit an amount of vertical compression and/or vertical cinching of the valve 800 when actuating the actuator 870. For example, a proximally directed force exerted on the tethers 873 of the actuator 870 can pull the distal end portion of the tethers 873 into and/or toward the waypoint catheter 877. With the waypoint catheter 877 disposed at, near, or below the subannular member 830 of the valve 800, the subannular member 830 of the valve 800 is pulled inward toward the end of the waypoint catheter 877 rather than inward and upward through the waypoint of the supra-annular member. As such, the waypoint catheter 877 can facilitate a “planar cinch” in which the subannular member 830 is cinched to reduce the perimeter and/or circumference, while limiting and/or minimizing an amount of axial compression/cinching of the transannular member (e.g., limiting and/or minimizing a reduction in height of the valve 800). In some instances, such a planar cinch can limit and/or substantially prevent pulling the subannular member 830 of the valve 800 up into the annulus while the valve 800 is being seated, which in turn, can limit and/or reduce undesirable contact between the proximal anchoring element 834 and native annular tissue when the actuator 870 and/or tethers 873 thereof release the proximal anchoring element 834 (e.g., allowing the proximal anchoring element 834 to return to or toward an unactuated, uncinched, and/or uncompressed configuration). Although not shown in FIGS. 34-37, the actuator 870 can also include one or more tethers that are, for example, disposed outside of the catheter 877 and configured to pull or actuate the proximal anchoring element 834 to return to or toward the unactuated, uncinched, and/or uncompressed configuration (e.g., as described above with reference to the actuator 370).

FIGS. 38-41 are various views of a prosthetic valve 900 removably coupled to a portion of a delivery/deployment system, according to an embodiment. The delivery/deployment system includes at least one actuator and/or control device 970 removably coupled to the valve 900 and configured to facilitate delivery and/or deployment of the valve 900 into a native annulus. The valve 900 can be similar to and/or substantially the same as any of the valves described herein (e.g., the valve 400). Accordingly, portions and/or aspects of the valve 900 are not described in further detail herein.

As shown in FIGS. 38 and 39, the valve 900 (or valve frame thereof) has a supra-annular member 920, a subannular member 930, and a transannular member 912 coupled therebetween. The supra-annular member 920 includes, forms, and/or defines a waypoint 928 configured to receive a portion of the actuator/control device 970 (referred to herein as “actuator 970” or “control device 970” interchangeably). The subannular member 930 forms a distal anchoring element 932 and a proximal anchoring element 934. The proximal anchoring element 934 is configured to transition (e.g., in response to actuation of the control device 970) between two or more configurations to facilitate delivery and/or deployment of the valve 900.

As described above with reference to the transannular member 412, the transannular member 912 includes a wire frame that is laser cut out of Nitinol or the like and, for example, heat-set into a desired shape and/or configuration. The transannular member 912 or wire frame thereof can include a set of struts 911 that form and/or define a set of compressible wire cells 913, as shown in FIG. 40. The struts form and/or define the compressible wire cells 913 such that an orientation and/or geometry thereof is/are substantially orthogonal to a central axis of the valve 900 to minimize strain along the struts 911 and/or any other portion of the wire frame when the transannular member 912 is in a delivery configuration (e.g., a compressed configuration and/or a cinched and compressed configuration, as described in further detail herein). Each strut 911 extends from an upper or supra-annular portion of the transannular member 912 to a lower or subannular portion and defines a portion of adjacent wire cells 913 (FIG. 40). The wire cells 913 can be substantially diamond-shaped.

Referring back to FIGS. 38 and 39, the actuator/control device 970 is shown coupled to the supra-annular member 920 of the valve 900. A guidewire catheter 984 and a portion of the actuator/control device 970 are shown extending through a waypoint 928 of the supra-annular member 920. The guidewire catheter 984 passes below the valve 900, through a guidewire attachment 935 along a distal edge or end portion of the valve 900, and through the distal anchoring element 932. The guidewire attachment 935 can be, for example, a suture or tether attached to the subannular member 930 that forms a loop or the like through which the guidewire catheter 984 extends. As described in further detail herein, such an arrangement can allow the guidewire catheter 984 to act as a quick release component for the actuator 970 after the valve 900 is seated in the annulus.

The actuator/control device 970 includes a set of tethers 973 configured to removably couple to one or more portions of the valve 900. For example, the control device 970 can include a first tether 973A, a second tether 973B, and a third tether 973C. The tethers 973 extend through the waypoint 928 of the supra-annular member 920 and/or are otherwise disposed below a drum or surface thereof. In this embodiment, the first tether 973A and the second tether 973B are shown as being routed through the waypoint 928 and a series of attachment points 946 (e.g., loops formed by sutures and/or the like) of the valve 900. More specifically, the valve 900 includes a series of attachment points 946 on an interior wall or surface that run along one of the struts 911 of the transannular member 912 that forms a set of adjacent wire cells 913 (e.g., rather than spanning across the empty space of the wire cells 913). In some instances, such a configuration can limit and/or can substantially prevent a cinching of the valve 900 along the central axis (e.g., cinching in a manner that reduces a height of the valve 900). As shown in FIGS. 38 and 39, the first tether 973A runs from the waypoint 928 and through attachment points 946 along a septal side of the transannular member 912 and the second tether runs from the waypoint 928 and through attachment point 946 along a freewall side of the transannular member 912.

In this embodiment, the first tether 973A is configured to cinch the subannular member 930 of the valve 900 in an antero-posterior direction. In other words, the first tether 973A is configured to cinch the subannular member 930 of the valve 900 by pulling the septal side and the freewall side of the subannular member 930 inward toward a longitudinal centerline of the valve 900. As shown, the first tether 973A is run along the septal side of the transannular member 912 (as described above), and through a set of attachment points 946 along the subannular member 930. In this embodiment, for example, the first tether 973A is run through four attachment points on the subannular member 930 that are arranged in a rectangular configuration. Moreover, a distal end portion of the first tether 973A forms a loop that is looped or disposed around the guidewire catheter 984 distal to the guidewire attachment 935 (e.g., around a portion of the guidewire catheter between the guidewire attachment 935 and the distal anchoring element 932). In this manner, the distal (looped) end of the first tether 973A is substantially anchored at a location at or near where the guidewire catheter 984 passes through the guidewire attachment 935. Thus, a proximally directed force exerted on the first tether 973A increases a tension along the first tether 973A that pulls the sides of the subannular member 930 (and corresponding sides of the transannular member 912) inward toward the longitudinal centerline of the valve 900 (e.g., based at least in part on the position and/or configuration of the set of anchoring points 946). Moreover, looping the distal end portion of the first tether 973A around the guidewire catheter 984 forms a “quick release” configuration and/or arrangement whereby retracting the guidewire catheter 984 to a position proximal to the guidewire attachment 935 releases and/or decouples the distal end portion of the first tether 973A.

In this embodiment, the second tether 973B is configured to cinch the proximal subannular anchoring element 934 of the valve 900. In other words, the second tether 973B is configured to cinch the subannular member 930 of the valve 900 by pulling, flipping, folding, and/or cinching the proximal anchoring element 934 inward toward and/or at least partially under the flow control component. As shown, the second tether 973B is run along an interior surface of the freewall side of the transannular member 912 (as described above), and through at least one attachment point 946 at or along the proximal anchoring element 934. In addition, a distal end portion of the second tether 973B forms a loop that is looped or disposed around the guidewire catheter 984 distal to the guidewire attachment 935 (e.g., around the portion of the guidewire catheter between the guidewire attachment 935 and the distal anchoring element 932). In this manner, the distal (looped) end of the second tether 973B is anchored at substantially the same location as the first tether 973A. Thus, a proximally directed force exerted on the second tether 973B increases a tension along the second tether 973B that pulls, flips, folds, and/or cinches the proximal anchoring element 934 inward in a distal direction toward and/or at least partially under the flow control component. Moreover, looping the distal end portion of the second tether 973B around the guidewire catheter 984 forms a “quick release” configuration and/or arrangement whereby retracting the guidewire catheter 984 to a position proximal to the guidewire attachment 935 releases and/or decouples the distal end portion of the second tether 973.

FIG. 40 is a side view of the valve 900 in an actuated and/or cinched configuration and/or state in which the sides of the subannular member 930 are pulled inward and the proximal anchoring element 934 is pulled distally. That is to say, the tethers 937A and 973B have been actuated, pulled, and/or placed in tension to actuate and/or cinch the subannular member 930 and the proximal anchoring element 934. While the proximal anchoring element 934 is shown and described as being pulled inward, pulled distally, pulled under the flow control component, etc., in some embodiments, the arrangement of at least the second tether 973B can be such that the proximal anchoring element 934 is pulled to a side of the transannular member 912 (e.g., a septal side or a freewall side) to place the proximal anchoring element 934 in the actuated and/or cinched configuration and/or state.

As described above, in some implementations, the subannular member 930 and the proximal anchoring element 934 can be transitioned to the cinched configuration after the valve is released from the distal end of the delivery catheter and allowed to expand to the expanded configuration. In other implementations, the subannular member 930 and the proximal anchoring element 934 can be transitioned to the cinched configuration prior to compressing the valve 900 to the compressed or delivery configuration. For example, after coupling the control device 970 to the valve 900, the tethers 973A and 973B can be actuated to increase a tension along the tethers 973A and 973B, which in turn, pulls, flips, folds, cinches, and/or otherwise transitions the subannular member 930 and the proximal anchoring element 934 (respectively) from the uncinched configuration to the cinched configuration. In some instances, once in the cinched configuration, the tethers 973A and 973B can be at least temporarily locked and/or secured (e.g., via a portion of the delivery system, control device 970, and/or the like) to at least temporarily maintain the subannular member 930 and the proximal anchoring element 934 in the cinched configuration. The valve 900 can then be folded and/or compressed in the lateral direction and compressed in the axial direction to place the valve 900 in the delivery configuration (e.g., a cinched and compressed configuration). The valve 900 in the delivery configuration can then be loaded into the delivery system and the control device 970 can be used to advance the valve 900 in the delivery configuration along the guidewire catheter 984 and through the lumen of the delivery catheter, as described above with reference to the delivery system and valve 300 and/or the delivery systems and valves described in the '032 PCT. In such implementations, the valve 900 can be configured to expand to the expanded or at least partially expanded configuration when released from the distal end of the delivery catheter while the subannular member 930 and proximal anchoring element 934 remain or substantially remain in the cinched configuration.

As described above, the arrangement of the tethers 973A and 973B being disposed within the transannular member 912 and run along the corresponding strut of the transannular member 912 can limit and/or can substantially prevent a cinching of the valve 900 along the central axis (e.g., cinching in a manner that reduces a height of the valve 900). As shown, the arrangement of the actuator 970 allows the valve 900 to be actuated and/or transitioned to a cinched state without substantially compressing the wire cells 913 of the transannular member 912 in the axial or transannular direction. In some instances, such a “planar cinch” can limit and/or substantially prevent the proximal anchoring element 934 from contacting annular tissue as the proximal anchoring element 934 transitions from its compressed, actuated, and/or cinched state to its uncompressed, unactuated, and/or uncinched state. In some instances, such contact may otherwise resist the valve 900 being seated in the annulus by pushing the proximal portion of the valve 900 out of the annulus in an atrial direction and/or may otherwise result in damage of the prosthetic valve 900 and/or the annular tissue. In some instances, such contact may limit and/or resist the proximal anchoring element 934 from returning to the uncompressed, unactuated, and/or uncinched state, which in turn, may result in improper, undesired, and/or unsecured seating of the valve 900 in the annulus.

As such, the actuator 970 shown in FIGS. 38-41 includes the third tether 973C that can be actuated, pulled, and/or placed in tension to aid proximal anchoring element 934 in transitioning from the first, cinched configuration and/or state to the second, uncinched configuration and/or state. For example, FIG. 40 shows the third tether 973C being directed and/or routed through and/or around at least the supra-annular member 920 and the transannular member 912, and removably coupled to the proximal anchoring element 934. Although not shown, the third tether 973C can be routed through any number of attachment points along any suitable portion of the valve 900. For example, the transannular member 912 can include one or more attachment points along an inner (interior) surface and/or an outer (exterior) surface through which the third tether 973C can be routed. As described above with reference to the first tether 973A and second tether 973B, the attachment points can be positioned such that the third tether 973C is routed, at least in part, along a strut of the transannular member 912, allowing the third tether 973C to actuate the proximal anchoring element 934 without substantially cinching the valve 900 in the axial direction.

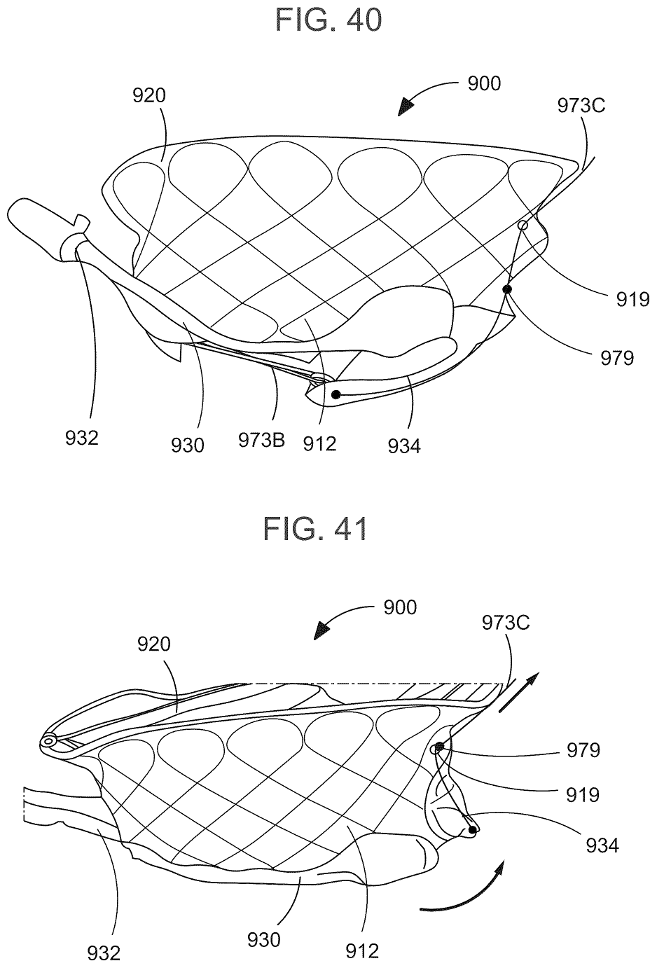

As shown in FIGS. 40 and 41, the transannular member 912 can include a waypoint 919 through which at least a portion of the third tether 973C can extend. In some implementations, the arrangement can be such that the third tether 973C passes below the supra-annular member 920 and not through, for example, the waypoint 928 of the supra-annular member 920. In other embodiments, the third tether 973C can pass through the waypoint 928 of the supra-annular member 920 alone or through both waypoints 928 and 919. FIG. 40 shows the third tether 973C being pulled or extended in a distal direction and/or otherwise being in a distal position or configuration when the proximal anchoring element 934 is cinched. As indicated by the arrows in FIG. 41, the third tether 973C can be actuated, pulled in a proximal direction, and/or otherwise placed in tension to transition or at least facilitate the transition of the proximal anchoring element 934 from the first, cinched configuration to the second, uncinched configuration. More specifically, a proximally directed force can be exerted on the third tether 973C that increases a tension along the third tether 973C and pulls, flips, and/or unfolds the proximal anchoring element 934 in a proximal direction to its uncinched and/or biased configuration and/or state.

The third tether 973C is also shown as including one or more locks 979 disposed along a length of the third tether 973C. The locks 979 can be, for example, knots, beads, bumps, protrusions, and/or any other suitable feature disposed at desired positions along the third tether 973C that correspond to one or more desired positions or configurations of the proximal anchoring element 934. Moreover, the waypoint 919 of the transannular member 912 can form and/or can be configured as a corresponding locking feature that can selectively engage the lock(s) 979 of the third tether 973C. For example, the third tether 973C is in a distal position when the proximal anchoring element 934 is in the first, cinched configuration (FIG. 40). In the distal position, the lock(s) 979 (or at least one lock 979) are/is distal to the waypoint 919 (e.g., locking feature) of the transannular member 912. FIG. 41 shows the third tether 973C being pulled in a proximal direction (e.g., to a proximal position) to pull or aid in the transition of the proximal anchoring element 934 to the second, uncinched, and/or biased configuration. In the proximal position, the lock(s) 979 (or at least one lock 979) are/is proximal to the waypoint 919 (e.g., locking feature) of the transannular member 912.

In some embodiments, arrangement of the lock(s) 979 and the waypoint 919 can be such that the lock(s) 979 is/are slightly larger than the waypoint 919 and can be at least partially compressible allowing the lock(s) 979 (e.g., having at least a slightly larger size) to be pulled through the waypoint 919 (e.g., the locking feature having at least a slightly smaller size). Thus, once the proximal anchoring element 934 is in the second configuration (or any other suitable configuration such as vertically or axially cinched configuration in a direction of the atrium), the lock(s) 979 can be pulled through the waypoint 919 to be disposed proximal thereto. As such, releasing the tension along the third tether 973C can be such that the lock(s) 979 of the third tether 973C contact a proximal side of the transannular member 912 forming the waypoint 919, thereby limiting and/or substantially preventing the proximal anchoring element 934 from moving back toward the first or cinched configuration.

Although the waypoint 919 is described as forming the corresponding locking feature configured to at least temporarily and/or selectively engage the lock(s) along the third tether 973C, in other embodiments, the locking feature can be separate from and/or independent of the waypoint 919 while performing substantially the same function. In still other embodiments, the third tether 973C need not include the lock(s) 979.

While the tethers 973A and 973B are described above as having a distal end that forms a loop allowing the distal ends to be disposed about a portion of the guidewire catheter 984, thereby collectively forming a “quick release” mechanism for temporarily anchoring the distal ends, the third tether 973C can similarly form and/or include a “quick release” mechanism, coupling, and/or arrangement with one or more features of the valve 900, the actuator 970, the guidewire catheter 984, and/or the like. For example, in some embodiments, the third tether 973C can include a first portion that removably or releasably couples to a second portion. The first portion of the third tether 973C can be routed through the delivery system such that a proximal end thereof is proximal to a delivery catheter and a distal end thereof is releasably or removably coupleable to the second portion of the third tether 973C. The second portion of the third tether 973C can be, for example, a distal portion that is coupled to the proximal anchoring element 934 (e.g., releasably or permanently) and that extends a relatively short distance from the proximal anchoring element 934 to releasably or removably couple to the first portion of the third tether 973C. For example, the second portion can include and/or can form one or more loops, couplers, connectors, etc. that can engage and/or removably couple to the first portion of the third tether 973C. Accordingly, the second portion of the third tether 973C can form a releasable connector, loop, suture, tether, etc. allowing the third tether 973C to be decoupled from the prosthetic valve 900, as described in detail in the '852 patent. In other embodiments, the third tether 973C can be configured to be routed through the attachment points, the proximal anchoring element 934, and the waypoint 919 such that both end of the third tether 973C are disposed outside of and proximal to the patient.

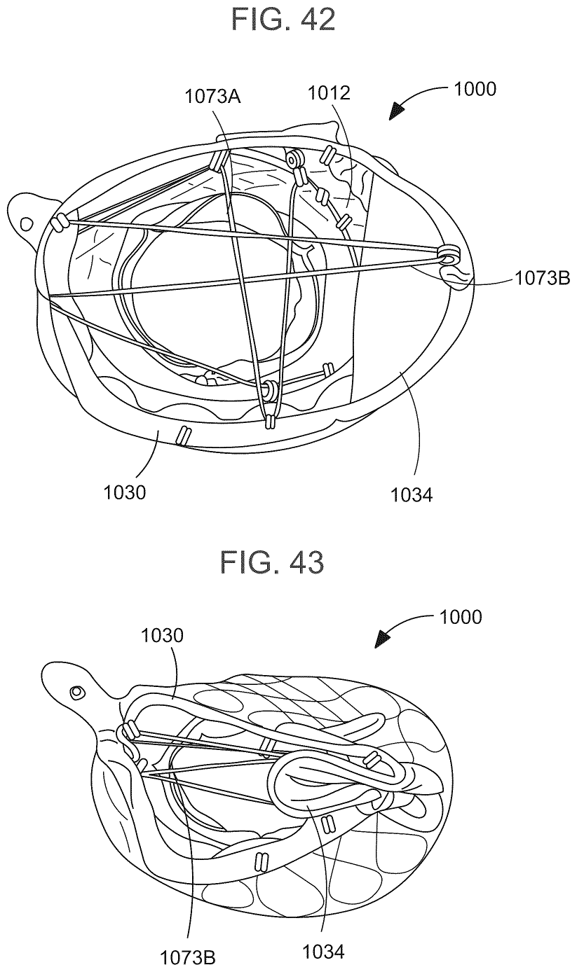

While the first and second tethers 973A and 973B are shown in FIGS. 38 and 39 as being routed through the attachment points in a particular manner, it should be understood that other methods of routing the tethers are possible. For example, FIGS. 42 and 43 are bottom views of a prosthetic valve 1000 removably coupled to a portion of a delivery/deployment system, according to an embodiment. The delivery/deployment system includes at least one actuator and/or control device 1070 removably coupled to the valve 1000 and configured to facilitate deployment of the valve 1000 into a native annulus. The valve 1000 and the actuator/control device 1070 can be similar to and/or substantially the same as any of the valves and/or actuators described herein (e.g., the valve 900 and actuator/control device 970, respectively). Accordingly, portions and/or aspects of the valve 1000 and portions and/or aspects of the actuator 1070 are not described in further detail herein.

The valve 1000 (or valve frame thereof) has a supra-annular member, a subannular member 1030, and a transannular member 1012 coupled therebetween. The transannular member 1012 includes a wire frame that is laser cut out of Nitinol or the like and, for example, heat-set into a desired shape and/or configuration. The transannular member 1012 or wire frame thereof includes a set of struts that form and/or define a set of compressible wire cells that are substantially diamond-shaped with an orientation and/or cell geometry substantially orthogonal to the central axis of the valve 1000, as described above with reference to the transannular member 912. The subannular member 1030 forms a proximal anchoring element 1034. Although not shown, the subannular member 1030 can include a guidewire attachment configured to receive a portion of a guidewire catheter, as described above with reference to the valve 900 shown in FIGS. 38-41.

The actuator 1070 includes a first tether 1073A and a second tether 1073B. FIG. 42 shows the tethers 1073A and 1073B being routed through a series of attachment points that run along a strut of the transannular member 1012 that forms a set of adjacent wire cells (e.g., rather than spanning across the empty space of the wire cells). In some instances, such a configuration can limit and/or can substantially prevent a cinching of the valve 1000 along the central axis (e.g., cinching in a manner that reduces a height of the valve 1000), as described in detail above. More specifically, in this embodiment, the first tether 1073A is routed through two attachment points along the subannular member 1030 rather than the four attachment points shown in FIGS. 38 and 39. In addition, the second tether 1073B is routed from the transannular member 1012 to a distal end of the valve 1000 (e.g., the guidewire coupler or other suitable attachment point at a distal end of the valve 1000), then to the proximal anchoring element 1034, and then back to the distal end of the valve 1000, rather than from the transannular member to the proximal anchoring element, and then to the distal end of the valve as shown in FIGS. 38 and 39.

As shown in FIG. 43, however, the specific routing of the tethers 1073A and 1073B allows the tethers 1073A and 1073B to cinch the subannular member 1030 of the valve 1000 without substantially compressing the wire cells of the transannular member 1012 in the axial or transannular direction. In some instances, such a “planar cinch” can limit and/or substantially prevent the proximal anchoring element 1034 from contacting annular tissue as the proximal anchoring element 1034 transitions from its compressed or actuated state to its uncompressed or unactuated state, as described in detail above with reference to the valve 900 shown in FIGS. 38-41. Although not shown in FIGS. 42 and 43, the actuator/control device 1070 can include a third tether and/or the like that can be actuated to transition and/or to aid in the transition of the proximal anchoring element 1034 from the first, cinched configuration to the second, uncinched configuration, as described above with reference to the third tether 973C.

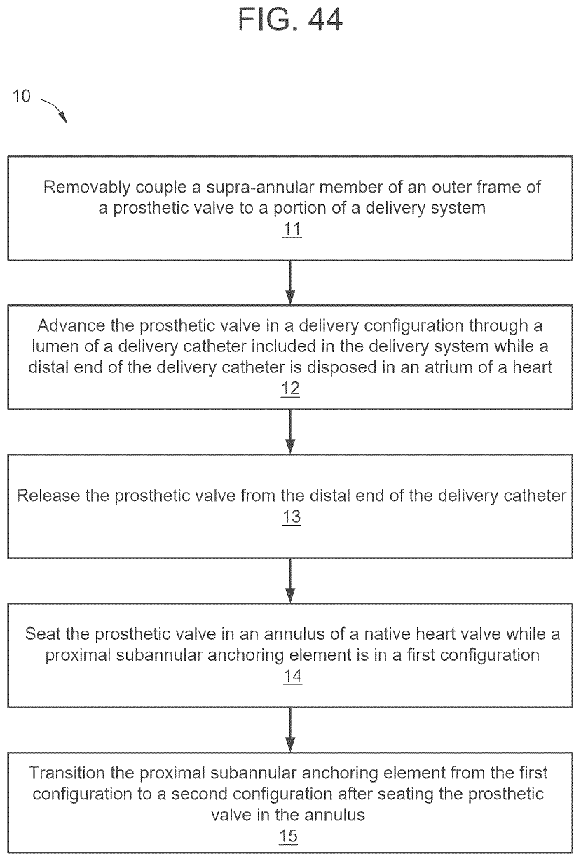

FIG. 44 is a flowchart illustrating a method 10 of deploying a side-deliverable transcatheter prosthetic valve according to an embodiment. The side-deliverable transcatheter prosthetic valve can be similar to and/or substantially the same as any of the prosthetic valves described herein. For example, the prosthetic valve can include an outer support frame and an (inner) flow control component that is mounted in and/or to the outer support frame. The outer support frame (or “valve frame”) can include, for example, a supra-annular member or region, a subannular member or region, and a transannular member or region coupled therebetween. The flow control component is mounted to the valve frame such that is extends through a portion of the transannular member or region, as described above.

The method 10 includes removably coupling the supra-annular member of the outer frame to a portion of a delivery system, at 11. For example, in some embodiments, the supra-annular member can include an attachment member or the like that can be used to temporarily couple the delivery system to the valve. In other embodiments, the supra-annular member can form and/or define an attachment point, waypoint, and/or any other suitable coupler that can removably couple to the portion of the delivery system.

The prosthetic valve in a delivery configuration is advanced through a lumen of a delivery catheter included in the delivery system while a distal end of the delivery catheter is disposed in an atrium of a heart, at 12. As described above with reference to the valve 100, the prosthetic valve can be placed into the delivery configuration and loaded into the lumen of the delivery catheter. In some instances, placing the valve into the delivery configuration can include, for example, folding the valve in a lateral direction or along a lateral axis and compressing the valve in an axial or blood flow direction or along a central axis of the valve. In some instances, the supra-annular member of the outer frame is removably coupled to the portion of the delivery system prior to being advanced through the lumen of the delivery catheter. In some such instances, for example, the portion of the delivery system can be used to advance the prosthetic valve in the delivery configuration through the lumen of the delivery catheter.

The prosthetic valve is released from the distal end of the delivery catheter, at 13. In some instances, the prosthetic valve can be partially released from the delivery catheter to allow a distal end portion of the valve (e.g., a distal anchoring element of the subannular member) to be inserted into the annulus of the native valve prior to fully releasing the valve. In other instances, the prosthetic valve can be fully released from the delivery catheter prior to inserting a portion of the prosthetic valve into the annulus. Moreover, the releasing of the prosthetic valve allows the released portion (or the valve in its entirety) to transition from the delivery configuration to an expanded or deployment configuration.

The prosthetic valve is seated in an annulus of a native heart valve while a proximal subannular anchoring element is in a first configuration, at 14. As described above with reference to specific embodiments, the proximal subannular anchoring element can be transitioned to the first or cinched configuration prior to being loaded into the and/or advanced through the delivery system or can be transitioned to the first or cinched configuration after being released from the distal end of the delivery catheter. The proximal subannular anchoring element can be placed in the first configuration in response to an actuation of an actuator removably coupled thereto. For example, the actuator can be one or more tethers that can be placed in tension to actuate, move, and/or otherwise place the proximal subannular anchoring element in the first configuration such that a perimeter and/or circumference of at least the subannular member is reduced to a size similar to or smaller than a perimeter and/or circumference of the annulus.

As described in detail above with respect to specific embodiments, the actuator and/or tether(s) thereof can be routed through one or more attachment points of the valve in such a manner that limits and/or substantially prevents a cinching of the valve along the central axis (e.g., cinching in a manner that reduces a height of the valve). In other embodiments, the tethers can be routed through a catheter or tube that extends through the supra-annular member such that an end portion is at, near, or past the subannular member of the valve frame, which can be operable in limiting and/or substantially preventing a cinching of the valve along the central axis. In some instances, such a “planar cinch” can limit and/or substantially prevent the proximal subannular anchoring element from contacting annular tissue as the proximal subannular anchoring element transitions from its compressed or actuated state to its uncompressed or unactuated state, as described in detail above.

After seating the prosthetic valve in the annulus, the proximal subannular anchoring element is transitioned from the first configuration to a second configuration, at 15. For example, in some implementations, the actuator can be actuated to move the proximal subannular anchoring element from the first configuration to the second configuration. In some implementations, a user or operator can reduce an amount of tension in one or more tethers allowing the proximal subannular anchoring element to return to a biased or expanded state or configuration. In some implementations, the actuator can be actuated such that the proximal subannular anchoring element is moved from the first (compressed) configuration, through an extended configuration, and to a cinched configuration, in which native tissue on a proximal side of the annulus is compressed or sandwiched between the proximal subannular anchoring element of the subannular member and a proximal portion of the supra-annular member, thereby securing the valve in the annulus. In some implementations, once the valve is seated and/or secured in the annulus of the native valve, the portion of the delivery system can be decoupled and/or removed from the valve and withdrawn from the body of the patient. In some implementations, the actuator can include a first portion or first tether that can be actuated to cinch the proximal subannular anchoring element and can include a second portion or second tether that can be actuated to return or aid the return of the proximal subannular anchoring element to its biased or uncinched configuration.

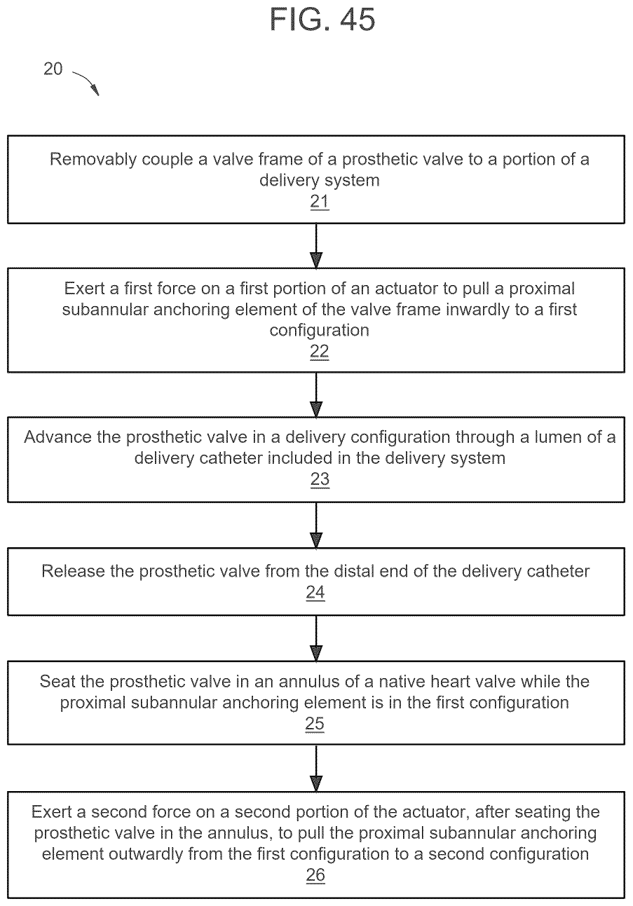

FIG. 45 is a flowchart illustrating a method 20 of deploying a side-deliverable prosthetic valve according to an embodiment. The side-deliverable prosthetic valve can be similar to and/or substantially the same as any of the prosthetic valves described herein. For example, the prosthetic valve can include an outer support frame and an (inner) flow control component that is mounted in and/or to the outer support frame. The outer support frame (or “valve frame”) can include, for example, a supra-annular member or region, a subannular member or region, and a transannular member or region coupled therebetween. The flow control component is mounted to the valve frame such that is extends through a portion of the transannular member or region, as described above.

The method 20 includes removably coupling the supra-annular member of the outer frame to a portion of a delivery system, at 21. For example, in some embodiments, the supra-annular member can include an attachment member or the like that can be used to temporarily couple the delivery system to the valve. In other embodiments, the supra-annular member can form and/or define an attachment point, waypoint, and/or any other suitable coupler that can removably couple to the portion of the delivery system. For example, the portion of the delivery system can include a connection member or yoke that is removably coupled to one or more attachment points on a drum of the supra-annular member, as described above with reference to specific embodiments.

A first force is exerted on a first portion of an actuator to pull a proximal subannular anchoring element of the valve frame inwardly to a first configuration, at 22. The proximal subannular anchoring element may be formed, at least in part, by the subannular member of the valve frame. The first portion of the actuator can be or can include one or more tethers that can be placed in tension in response to an applied force. A distal end portion of the tether(s) can engage and/or can be removably coupled to the proximal subannular anchoring element while a proximal end portion of the tether(s) are disposed outside of the body, allowing the tether(s) to be manipulated by a user (e.g., a doctor, physician, technician, surgeon, etc.). For example, the first force can be a proximally directed force exerted on a proximal end portion of the one or more tethers, thereby placing the tether(s) in tension. The tension along the tether(s), in turn, actuates, moves, pulls, and/or otherwise transitions the proximal subannular anchoring element between any number of configurations. In this implementation, the first configuration is a compressed or cinched configuration in which the proximal subannular anchoring element is pulled, folded, moved, and/or otherwise reconfigured in an inward direction (e.g., in a direction toward a central axis of the prosthetic valve). As such, a perimeter and/or circumference of at least the subannular member of the valve frame is reduced to a size similar to or smaller than a perimeter and/or circumference of an annulus of a native heart valve.

As described in detail above with respect to specific embodiments, the actuator and/or first portion (e.g., tether(s)) thereof can be routed through one or more attachment points of the valve frame in such a manner that limits and/or substantially prevents a cinching of the valve along the central axis (e.g., cinching in a manner that reduces a height of the valve). For example, the first portion or tether(s) of the actuator can be routed through a waypoint of the supra-annular member and through any number of attachment points mounted to an interior surface of the transannular member and along a strut from a set of struts forming a portion of the transannular member. In other embodiments, the tethers can be routed through a catheter or tube that extends through the supra-annular member such that an end portion is at, near, or past the subannular member of the valve frame, which can be operable in limiting and/or substantially preventing a cinching of the valve along the central axis. In some instances, such a “planar cinch” can limit and/or substantially prevent compression of the transannular member along the central axis that may otherwise cause the proximal subannular anchoring element to contact or snag on annular tissue as the proximal subannular anchoring element is transitioned from its on or more configurations, states, positions, etc., as described in detail above.

The prosthetic valve is advanced through a lumen of a delivery catheter included in the delivery system while a distal end of the delivery catheter is disposed in an atrium of a heart, at 23. As described above with reference to the valve 100, the prosthetic valve can be placed into a delivery configuration and loaded into the lumen of the delivery catheter. In some instances, placing the valve in the delivery configuration can include, for example, folding the valve in a lateral direction or along a lateral axis and compressing the valve in an axial or blood flow direction or along a central axis of the prosthetic valve. In some instances, the supra-annular member of the outer frame is removably coupled to the portion of the delivery system prior to being advanced through the lumen of the delivery catheter, allowing the portion of the delivery system to be used to advance the prosthetic valve in the delivery configuration through the lumen of the delivery catheter. As described above with reference to specific embodiments, the proximal subannular anchoring element can be pulled to the first configuration prior to the prosthetic valve being compressed and loaded into and/or advanced through the delivery system.

The prosthetic valve is released from the distal end of the delivery catheter, at 24. In some instances, the prosthetic valve can be partially released from the delivery catheter to allow a distal end portion of the valve (e.g., a distal anchoring element of the subannular member) to be inserted into the annulus of the native valve prior to fully releasing the valve. In other instances, the prosthetic valve can be fully released from the delivery catheter prior to inserting a portion of the prosthetic valve into the annulus. Moreover, the releasing of the prosthetic valve allows the released portion (or the prosthetic valve in its entirety) to transition from the delivery configuration to an expanded or deployment configuration. In some implementations, the proximal subannular anchoring element may be maintained in the first or compressed configuration while the prosthetic valve is released from the delivery catheter.

The method 20 further includes seating the prosthetic valve in the annulus of the native heart valve while the proximal subannular anchoring element is in the first configuration, at 25. As described above with reference to specific embodiments, with the proximal subannular anchoring element in the first or cinched configuration, a perimeter and/or circumference of at least the subannular member is reduced to a size similar to or smaller than a perimeter and/or circumference of the annulus. The reduced perimeter and/or circumference of the subannular member allows at least a portion of the prosthetic valve to be “dropped” into the annulus and/or otherwise moved through the annulus. Moreover, cinching and/or transitioning the proximal subannular anchoring element into the first configuration without substantially compressing the transannular member of the valve frame (e.g., the “planar cinch” arrangement) limits and/or reduces a likelihood of improperly seating the prosthetic valve due to the subannular member (or a portion thereof) being in the annulus rather than below the annulus.

After seating the prosthetic valve in the annulus, a second force is exerted on a second portion of the actuator to pull the proximal subannular anchoring element outwardly from the first configuration to a second configuration, at 26. For example, the second portion of the actuator can be or can include one or more tethers that can be placed in tension in response to an applied force. A distal end portion of the tether(s) can engage and/or can be removably coupled to the proximal subannular anchoring element while a proximal end portion of the tether(s) are disposed outside of the body, allowing the tether(s) to be manipulated by a user (e.g., a doctor, physician, technician, surgeon, etc.). For example, the second force can be a proximally directed force exerted on a proximal end portion of the one or more tethers, thereby placing the tether(s) in tension. The tension along the tether(s), in turn, actuates, moves, pulls, and/or otherwise transitions the proximal subannular anchoring element between any number of configurations. In this implementation, the second configuration is an extended or uncinched configuration in which the proximal subannular anchoring element is pulled, unfolded, moved, and/or otherwise reconfigured in an outward direction (e.g., in a direction away from the central axis of the prosthetic valve). As such, a perimeter and/or circumference of at least the subannular member of the valve frame is increased, for example, to a size that is larger than the perimeter and/or circumference of the annulus.

While the first portion of the actuator (e.g., tether(s)) are described above as being routed through one or more inner attachment points (e.g., attachment points along an interior surface of the transannular member), the second portion of the actuator (e.g., tether(s)) can be routed through one or more portion of the prosthetic valve and at least one attachment point mounted to an exterior surface of the transannular member (or proximal subannular anchoring element). For example, the second portion of the actuator can be and/or can include one or more tether(s) that are selectively routed through the valve such that a proximally directed force exerted on the tether(s) (e.g., the second force) pulls the proximal subannular anchoring element in a direction away from the central axis of the valve (e.g., outwardly).

In some implementations, the second portion of the actuator can be actuated such that the proximal subannular anchoring element is moved from the first (compressed) configuration, through an extended configuration, and to a cinched configuration (e.g., second configuration), in which native tissue on a proximal side of the annulus is compressed or sandwiched between the proximal subannular anchoring element of the subannular member and a proximal portion of the supra-annular member, thereby securing the valve in the annulus. In some implementations, once the valve is seated and/or secured in the annulus of the native valve, the portion of the delivery system can be decoupled and/or removed from the valve and withdrawn from the body of the patient.

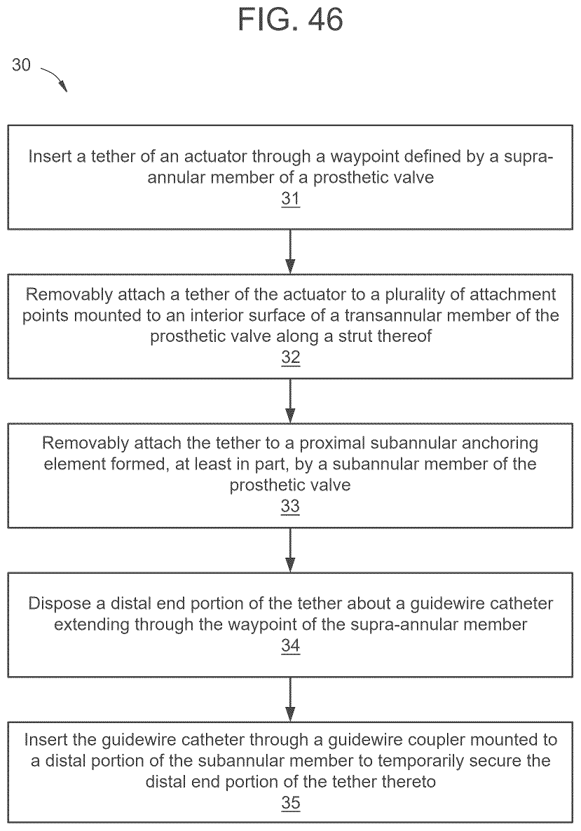

FIG. 46 is a flowchart illustrating a method 30 of coupling an actuator to a valve frame of a side-deliverable prosthetic valve according to an embodiment. The side-deliverable prosthetic valve can be similar to and/or substantially the same as any of the prosthetic valves described herein. For example, the prosthetic valve can include an outer support frame and an (inner) flow control component that is mounted in and/or to the outer support frame. The outer support frame (or “valve frame”) can include, for example, a supra-annular member or region, a subannular member or region, and a transannular member or region coupled therebetween. The flow control component is mounted to the valve frame such that is extends through a portion of the transannular member or region, as described above.

The method 30 includes inserting a tether of the actuator through a waypoint defined by the supra-annular member of the valve frame, at 31. For example, the supra-annular member includes a drum or the like that covers or forms a surface on a wire frame portion of the supra-annular member. The supra-annular member further includes and/or defines an opening or waypoint in, along, or through the drum that can allow one or more components to be passed from an exterior (upper) side of the supra-annular member to an interior (lower) side of the supra-annular member.

The tether is removably attached to a set of attachment points mounted to an interior surface of the transannular member along a strut from a set of struts, at 32. For example, the transannular member can include and/or can be formed by a wire frame that is covered by a biocompatible material (e.g., a wire frame that is laser cut from a tube or sheet of a shape-memory material). More specifically, the transannular member or wire frame thereof can include a set of struts that form and/or define a set of compressible wire cells, as described above with reference to the transannular member 912. The struts form and/or define the compressible wire cells such that an orientation and/or geometry thereof is/are substantially orthogonal to a central axis of the prosthetic valve to minimize strain along the struts and/or any other portion of the wire frame (e.g., associated with compressing and/or cinching the prosthetic valve). In some embodiments, each strut can extend at an angle from an upper or supra-annular portion of the transannular member to a lower or subannular portion and can define a portion of adjacent wire cells that are, for example, substantially diamond-shaped. In some embodiments, at least one of the attachment points is distal to the proximal subannular anchoring element.

The tether is removably attached to a proximal subannular anchoring element formed, at least in part, by the subannular member of the valve frame of the prosthetic valve, at 33. For example, the proximal subannular anchoring element can include and/or can form an attachment point to which the tether is removably attached. In some embodiments, the attachment points along the strut and the attachment point(s) of the proximal subannular anchoring element are loops through which the tether can extend and/or can be routed. In other embodiments, the attachment points can be any suitable attachment, coupler, anchor, tie, etc. that can secure the tether against one or more surfaces of the prosthetic valve or valve frame while allowing the tether to be moved in a proximal direction and/or a distal direction (e.g., advanced or retracted).

A distal end portion of the tether is disposed about a guidewire catheter that extends through the waypoint of the supra-annular member, at 34. The guidewire catheter can be similar to any of the guidewire catheters described above. As described above with reference to the guidewire catheter 984, the guidewire catheter can extend through the waypoint such that a portion of the guidewire catheter is below the flow control component of the prosthetic valve. After disposing the distal end portion of the tether about the guidewire catheter, a portion of the guidewire catheter distal to the tether can be inserted through a guidewire coupler mounted to a distal portion of the subannular member to at least temporarily secure the distal end portion of the tether to the distal portion of the subannular member, at 35.

As described above with reference to specific embodiments, looping and/or disposing the distal end portion of the tether around/about the guidewire catheter anchors the distal end portion of the tether. Moreover, the arrangement of the attachment points along the interior surface of the transannular member can be such that at least one attachment point is distal to the proximal subannular anchoring element. In this manner, the attachment point anchors a portion of the tether before or proximal to the proximal subannular anchoring element and the guidewire catheter anchors a portion of the tether after or distal to the proximal subannular anchoring element. As such, a proximally directed force exerted on the tether acts to pull the proximal subannular anchoring element (by virtue of the tether being routed through its attachment point) inward toward the central axis of the prosthetic valve. In some implementations, the proximal subannular anchoring element can be pulled to a position in which the attachment point of the proximal subannular anchoring element is between the distal most attachment point along the interior surface of the transannular member and the guidewire catheter. In some embodiments, the attachment point of the proximal subannular anchoring element, the distal most anchoring element along the interior surface of the transannular member, and the portion of the guidewire catheter about which the tether is disposed can be in a substantially similar position along the central axis of the prosthetic valve (e.g., substantially coplanar). As described above with reference to specific embodiments, the coupling of the actuator to the prosthetic valve according to the method 30 can allow the proximal subannular anchoring element to be actuated, cinched, and/or otherwise reconfigured without substantially compressing the transannular member in a direction along the central axis (e.g., without substantially reducing a height of the transannular member).

While various schematics, embodiments, and/or implementations have been described above, it should be understood that they have been presented by way of example only, and not limitation. Likewise, it should be understood that the specific terminology used herein is for the purpose of describing particular embodiments and/or features or components thereof and is not intended to be limiting. Various modifications, changes, and/or variations in form and/or detail may be made without departing from the scope and/or spirit of the disclosure and/or without altering the function and/or advantages thereof unless expressly stated otherwise. Functionally equivalent embodiments, implementations, and/or methods, in addition to those enumerated herein, will be apparent to those skilled in the art from the foregoing descriptions and are intended to fall within the scope of the disclosure. While embodiments (and/or features, components, configurations, aspects, etc. thereof) may be described above in the context of certain implementations, it should be understood that such implementations are presented by way of example only, and not limitation. Any of the embodiments (and/or features, components, configurations, aspects, etc. thereof) can be used in, and/or adapted for use in, other implementations unless expressly stated otherwise.

The specific configurations of the various components can also be varied. For example, the size and specific shape of the various components can be different from the embodiments shown, while still providing the functions as described herein. More specifically, the size and shape of the various components can be specifically selected for a desired or intended usage. Thus, it should be understood that the size, shape, and/or arrangement of the embodiments and/or components thereof can be adapted for a given use unless the context explicitly states otherwise.

Where schematics, embodiments, and/or implementations described above indicate certain components arranged in certain orientations, configurations, or positions, the arrangement of components may be modified. Although various embodiments have been described as having particular features, configurations, and/or combinations of components, other embodiments are possible having a combination of any features, configurations, and/or components from any of embodiments described herein, except mutually exclusive combinations. The embodiments described herein can include various combinations and/or sub-combinations of the functions, components, configurations, and/or features of the different embodiments described.

Where methods described above indicate certain events, steps, and/or procedures occurring in certain order, the ordering of certain events, steps, and/or procedures may be modified. Additionally, certain of the events, steps, and/or procedures may be performed concurrently in a parallel process, when possible, as well as performed sequentially as described above. While methods have been described as having particular steps and/or combinations of steps, other methods are possible having a combination of any steps from any of methods described herein, except mutually exclusive combinations and/or unless the context clearly states otherwise.

What is claimed:

1. A side-deliverable prosthetic valve, the prosthetic valve comprising:
a valve frame defining a central channel extending along a central axis of the prosthetic valve, the valve frame including a proximal subannular anchoring element;
a flow control component mounted within the central channel and configured to permit blood flow therethrough in a direction along the central axis; and
an actuator configured to be removably attached to the proximal subannular anchoring element, the actuator including a first portion removably attached to an interior surface of the valve frame in at least one location distal to the proximal subannular anchoring element, the first portion configured, in response to a proximally directed force, to pull the proximal subannular anchoring element inwardly toward the central axis to a first configuration,
the actuator including a second portion removably attached, at least in part, to an exterior surface of the valve frame, the second portion configured, in response to a proximally directed force, to pull the proximal subannular anchoring element outwardly away from the central axis from the first configuration to a second configuration.

2. The prosthetic valve of claim 1, wherein the prosthetic valve is compressible along the central axis and a lateral axis perpendicular to the central axis to place the prosthetic valve in a compressed configuration for side-delivery into a chamber of a heart via a delivery catheter,
wherein each of the central axis and the lateral axis is perpendicular to a longitudinal axis of the delivery catheter when the prosthetic valve is in the compressed configuration within the delivery catheter, and
wherein the prosthetic valve is configured to transition from the compressed configuration to an expanded configuration when the prosthetic valve is released from the delivery catheter into the chamber of the heart.

3. The prosthetic valve of claim 2, wherein the valve frame includes a supra-annular member, a subannular member, and a transannular member coupled therebetween, the transannular member defining the central channel and including a plurality of wire struts that define a plurality of diamond-shaped cells, the diamond-shaped cells having an orientation that allows compression of the prosthetic valve along the central axis.

4. The prosthetic valve of claim 1, wherein the valve frame includes a supra-annular member, a subannular member, and a transannular member coupled therebetween, the transannular member defining the central channel and including a plurality of wire struts that define a plurality of diamond-shaped cells,
the first portion of the actuator includes a tether removably attached to the proximal subannular anchoring element, a portion of the tether disposed in the central channel and removably attached to the interior surface along a strut from the plurality of struts.

5. The prosthetic valve of claim 4, wherein the portion of the tether being removably attached to the interior surface is such that the proximally directed force causes the tether to pull the proximal subannular anchoring element inwardly toward the central axis substantially without compressing the transannular member in a direction along the central axis.

6. The prosthetic valve of claim 1, wherein the valve frame includes a supra-annular member, a subannular member, and a transannular member coupled therebetween, the proximal subannular anchoring element being formed, at least in part, by a proximal portion of the subannular member.

7. The prosthetic valve of claim 6, wherein the first portion of the actuator pulling the proximal subannular anchoring element inwardly toward the central axis to the first configuration reduces a perimeter of the subannular member to facilitate seating the prosthetic valve in an annulus of a native heart valve.

8. The prosthetic valve of claim 7, wherein the second portion of the actuator pulling the proximal subannular anchoring element outwardly away from the central axis to the second configuration increases the perimeter of the subannular member such that the perimeter of the subannular member is larger than a perimeter of the annulus of the native heart valve.

9. A method of deploying a side-deliverable prosthetic valve in an annulus of a native heart valve, the prosthetic valve having (i) a valve frame with a supra-annular member, a subannular member, and a transannular member coupled therebetween and (ii) a flow control component mounted to the valve frame and at least partially disposed in the transannular member, the method comprising:
removably coupling the valve frame to a portion of a delivery system;
exerting a first force on a first portion of an actuator to pull a proximal subannular anchoring element of the subannular member inwardly to a first configuration;
advancing the prosthetic valve in a delivery configuration through a lumen of a delivery catheter included in the delivery system, a distal end of the delivery catheter being disposed in an atrium of the heart;
releasing the prosthetic valve from the distal end of the delivery catheter;
seating the prosthetic valve in the annulus of the native heart valve while the proximal subannular anchoring element is in the first configuration; and
exerting a second force on a second portion of the actuator, after seating the prosthetic valve in the annulus, to pull the proximal subannular anchoring element outwardly from the first configuration to a second configuration.

10. The method of claim 9, wherein after exerting the first force to pull the proximal subannular anchoring element to the first configuration and before advancing the prosthetic valve through the lumen of the delivery catheter, the method further comprising:
compressing the prosthetic valve in a first direction along a lateral axis of the prosthetic valve and a second direction along a central axis of the prosthetic valve to place the prosthetic valve in the delivery configuration, the lateral axis being perpendicular to the central axis; and
loading the prosthetic valve in the delivery configuration into the lumen of the delivery catheter such that a longitudinal axis of the prosthetic valve is substantially parallel to a longitudinal axis of the delivery catheter,
wherein the longitudinal axis of the prosthetic valve is perpendicular to each of the central axis and the lateral axis of the prosthetic valve.

11. The method of claim 9, wherein the proximal subannular anchoring element is formed, at least in part, by a proximal portion of the subannular member,
the proximal subannular anchoring element in the first configuration is such that a perimeter of the subannular member is less than a perimeter of the annulus of the native heart valve, and
the proximal subannular anchoring element in the second configuration is such that the perimeter of the subannular member is larger than the perimeter of the annulus of the native heart valve.

12. The method of claim 9, wherein the first portion of the actuator includes a first tether, and the second portion of the actuator includes a second tether, the method further comprising:
releasably coupling the first tether to the proximal subannular anchoring element, the first force being a proximally directed force on the first tether that causes the first tether to pull the proximal subannular anchoring element inwardly toward a central axis of the prosthetic valve; and
releasably coupling the second tether to the proximal subannular anchoring element, the second force being a proximally directed force on the second tether that causes the second tether to pull the proximal subannular anchoring element outwardly away from the central axis of the prosthetic valve.

13. The method of claim 12, wherein the transannular member includes a plurality of wire struts that define a plurality of diamond-shaped cells, the diamond-shaped cells having an orientation that allows compression of the prosthetic valve in a direction along the central axis.

14. The method of claim 13, wherein a portion of the first tether extends through the transannular member and is releasably attached to an interior surface of the transannular member along a strut from the plurality of struts.

15. The method of claim 14, wherein the portion of the first tether is releasably attached to the interior surface of the transannular member in at least one location distal to the proximal subannular anchoring element.

16. The method of claim 14, wherein the portion of the first tether being releasably attached to the interior surface along the strut is such that exerting the proximally directed force on the first tether pulls the proximal subannular anchoring element inwardly toward the central axis substantially without compressing the transannular member in the direction along the central axis.

17. The method of claim 12, further comprising:
locking the proximal subannular anchoring element in the second configuration.

18. The method of claim 17, wherein locking the proximal subannular anchoring element includes pulling the second tether in a proximal direction such that a lock along the second tether engages a portion of the valve frame.

19. A method of coupling an actuator to a valve frame of a side-deliverable prosthetic valve, the valve frame having a supra-annular member, a subannular member, and a transannular member coupled therebetween, the transannular member including a plurality of wire struts that define a plurality of diamond-shaped cells, the method comprising:
inserting a tether of the actuator through a waypoint defined by the supra-annular member;
removably attaching the tether to a plurality of attachment points mounted to an interior surface of the transannular member along a strut from the plurality of struts;
removably attaching the tether to a proximal subannular anchoring element formed, at least in part, by the subannular member of the valve frame;
disposing a distal end portion of the tether about a guidewire catheter extending through the waypoint of the supra-annular member; and
inserting the guidewire catheter through a guidewire coupler mounted to a distal portion of the subannular member to temporarily secure the distal end portion of the tether to the distal portion of the subannular member.

20. The method of claim 19, wherein the distal end portion of the tether forms a loop allowing the distal end portion of the tether to be disposed about the guidewire catheter.

21. The method of claim 19, wherein the diamond-shaped cells of the transannular member have a geometry and orientation that allows the prosthetic valve to be compressed along a central axis of the prosthetic valve.

22. The method of claim 21, wherein the transannular member includes at least one proximal hinge point and at least one distal hinge point collectively configured to allow the prosthetic valve to be compressed along a lateral axis of the prosthetic valve, the lateral axis being perpendicular to the central axis, and
wherein compressing the prosthetic valve in a first direction along the lateral axis and a second direction along the central axis places the prosthetic valve in a delivery configuration for side delivery into a chamber of a heart via a delivery catheter.

23. The method of claim 22, wherein coupling the actuator to the valve frame is such that (i) exerting a proximally directed force on the tether causes the tether to pull the proximal subannular anchoring element inwardly toward a central axis of the prosthetic valve substantially without compressing the transannular member in a direction along the central axis, and (ii) releasing the proximally directed force on the tether allows the proximal subannular anchoring element to move outwardly away from the central axis.

24. The method of claim 19, wherein the tether is a first tether of the actuator, and the strut is a first strut from the plurality of struts, the method further comprising:
inserting a second tether of the actuator through the waypoint;
removably attaching the second tether to a plurality of attachment points mounted to the interior surface of the transannular member along a second strut from the plurality of struts;
removably attaching the second tether to at least one attachment point mounted to an anterior side of the subannular member and at least one attachment point mounted to a posterior side of the subannular member; and
disposing a distal end portion of the second tether about the guidewire catheter prior to inserting the guidewire catheter through the guidewire coupler to temporarily secure the distal end portion of the second tether to the distal portion of the subannular member.

25. The method of claim 24, wherein coupling the actuator to the valve frame is such that (i) exerting a proximally directed force on the second tether causes the second tether to pull the anterior side of the subannular member and the posterior side of the subannular member inwardly toward a central axis of the prosthetic valve, and (ii) releasing the proximally directed force on the second tether allows the anterior side and the posterior side of the subannular member to move outwardly away from the central axis.

26. The method of claim 19, wherein the tether is a first tether of the actuator, the method further comprising:
inserting a second tether of the actuator through the waypoint;
removably attaching the second tether to at least one attachment point mounted to an exterior surface of the transannular member; and
removably attaching the second tether to the proximal subannular anchoring element.

27. The method of claim 26, wherein coupling the actuator to the valve frame is such that (i) exerting a proximally directed force on the first tether causes the tether to pull the proximal subannular anchoring element inwardly toward a central axis of the prosthetic valve substantially without compressing the transannular member in a direction along the central axis, and (ii) exerting a proximally directed force on the second tether causes the second tether to pull the proximal subannular anchoring element to move outwardly away from the central axis.
